SEQUENCE LISTING (53 sequences)

SEQ ID NO: 1 - protein, 123 aa, synthetic construct
EVQLVQSGAEVKKPGASVKVSCKASGYTFTGYYMNWVRQAPGQGLEWMGEINPATGSTTYNQKFKARVTITRDTSTSTAYMELSSLRSEDTAVYYCARRDYYYGSGFYAMDYWGQGTLVTVSS

SEQ ID NO: 2 - DNA, 369 bp, synthetic construct
  source 1..369: mol_type = other DNA
gaggtgcagctggtgcagagcggcgccgaggtgaagaagcccggcgccagcgtgaaggtgagctgcaaggccagcggctacaccttcaccggctactacatgaactgggtgcggcaggcccccggccagggcctggagtggatgggcgagatcaaccccgccaccggcagcaccacctacaaccagaagttcaaggcccgggtgaccatcacccgggacaccagcaccagcaccgcctacatggagctgagcagcctgcggagcgaggacaccgccgtgtactactgcgcccggcgggactactactacggcagcggcttctacgctatggactactggggccagggcaccctggtgaccgtgagcagc

SEQ ID NO: 3 - protein, 106 aa, synthetic construct
DIQMTQSPSSLSASVGDRVTITCQASQSVSNNLNWYQQKPGKAPKLLIYGASKLEDGVPSRFSGSGSGTDFTFTISSLQPEDFATYYCLQHRYLWTFGQGTKVEIK

SEQ ID NO: 4 - DNA, 318 bp, synthetic construct
  source 1..318: mol_type = other DNA
gacatccagatgacccagagccccagcagcctgagcgccagcgtgggcgaccgggtgaccatcacctgccaggccagccagagcgtgagcaacaacctgaactggtaccagcagaagcccggcaaggcccccaagctgctgatctacggcgccagcaagctggaggacggcgtgcccagccggttcagcggcagcggcagcggcaccgacttcaccttcaccatcagcagcctgcagcccgaggacttcgccacctactactgcctgcagcaccggtacctgtggaccttcggccagggcaccaaggtggagatcaag

SEQ ID NO: 5 - protein, 123 aa, synthetic construct
EVQLVQSGAEVKKPGASVKVSCKASGYTFTGYYMNWVRQAPGQGLEWMGEINPATGSTTYNQKFKARVTITRDTSTSTAYMELSSLRSEDTAVYYCARRDYYLGSNSYALDYWGQGTLVTVSS

SEQ ID NO: 6 - DNA, 369 bp, synthetic construct
  source 1..369: mol_type = other DNA
gaggtgcagctggtgcagagcggcgccgaggtgaagaagcccggcgccagcgtgaaggtgagctgcaaggccagcggctacaccttcaccggctactacatgaactgggtgcggcaggcccccggccagggcctggagtggatgggcgagatcaaccccgccaccggcagcaccacctacaaccagaagttcaaggcccgggtgaccatcacccgggacaccagcaccagcaccgcctacatggagctgagcagcctgcggagcgaggacaccgccgtgtactactgcgcccggcgggactactacctcggcagcaactcctacgcgctggactactggggccagggcaccctggtgaccgtgagcagc

SEQ ID NO: 7 - protein, 106 aa, synthetic construct
DIQMTQSPSSLSASVGDRVTITCQASQSVSNNLNWYQQKPGKAPKLLIYGASKLEDGVPSRFSGSGSGTDFTFTISSLQPEDFATYYCLQHRYMWTFGQGTKVEIK

SEQ ID NO: 8 - DNA, 318 bp, synthetic construct
  source 1..318: mol_type = other DNA
gacatccagatgacccagagccccagcagcctgagcgccagcgtgggcgaccgggtgaccatcacctgccaggccagccagagcgtgagcaacaacctgaactggtaccagcagaagcccggcaaggcccccaagctgctgatctacggcgccagcaagctggaggacggcgtgcccagccggttcagcggcagcggcagcggcaccgacttcaccttcaccatcagcagcctgcagcccgaggacttcgccacctactactgcctgcagcaccggtatatgtggaccttcggccagggcaccaaggtggagatcaag

SEQ ID NO: 9 - protein, 106 aa, synthetic construct
DIQMTQSPSSLSASVGDRVTITCQASQSVSNNLNWYQQKPGKAPKLLIYGASKLEDGVPSRFSGSGSGTDFTFTISSLQPEDFATYYCQQHRFMWTFGQGTKVEIK

SEQ ID NO: 10 - DNA, 318 bp, synthetic construct
  source 1..318: mol_type = other DNA
gacatccagatgacccagagccccagcagcctgagcgccagcgtgggcgaccgggtgaccatcacctgccaggccagccagagcgtgagcaacaacctgaactggtaccagcagaagcccggcaaggcccccaagctgctgatctacggcgccagcaagctggaggacggcgtgcccagccggttcagcggcagcggcagcggcaccgacttcaccttcaccatcagcagcctgcagcccgaggacttcgccacctactactgccagcagcaccggttcatgtggaccttcggccagggcaccaaggtggagatcaag

SEQ ID NO: 11 - protein, 106 aa, synthetic construct
DIQMTQSPSSLSASVGDRVTITCQASQSVSNNLNWYQQKPGKAPKLLIYGASKLEDGVPSRFSGSGSGTDFTFTISSLQPEDFATYYCQQHRYMWTFGQGTKVEIK

SEQ ID NO: 12 - DNA, 318 bp, synthetic construct
  source 1..318: mol_type = other DNA
gacatccagatgacccagagccccagcagcctgagcgccagcgtgggcgaccgggtgaccatcacctgccaggccagccagagcgtgagcaacaacctgaactggtaccagcagaagcccggcaaggcccccaagctgctgatctacggcgccagcaagctggaggacggcgtgcccagccggttcagcggcagcggcagcggcaccgacttcaccttcaccatcagcagcctgcagcccgaggacttcgccacctactactgccagcagcacaggtacatgtggaccttcggccagggcaccaaggtggagatcaag

SEQ ID NO: 13 - protein, 244 aa, synthetic construct
EVQLVQSGAEVKKPGASVKVSCKASGYTFTGYYMNWVRQAPGQGLEWMGEINPATGSTTYNQKFKARVTITRDTSTSTAYMELSSLRSEDTAVYYCARRDYYYGSGFYAMDYWGQGTLVTVSSGGGGSGGGGSGGGGSDIQMTQSPSSLSASVGDRVTITCQASQSVSNNLNWYQQKPGKAPKLLIYGASKLEDGVPSRFSGSGSGTDFTFTISSLQPEDFATYYCLQHRYLWTFGQGTKVEIK

SEQ ID NO: 14 - protein, 244 aa, synthetic construct
EVQLVQSGAEVKKPGASVKVSCKASGYTFTGYYMNWVRQAPGQGLEWMGEINPATGSTTYNQKFKARVTITRDTSTSTAYMELSSLRSEDTAVYYCARRDYYLGSNSYALDYWGQGTLVTVSSGGGGSGGGGSGGGGSDIQMTQSPSSLSASVGDRVTITCQASQSVSNNLNWYQQKPGKAPKLLIYGASKLEDGVPSRFSGSGSGTDFTFTISSLQPEDFATYYCLQHRYLWTFGQGTKVEIK

SEQ ID NO: 15 - protein, 244 aa, synthetic construct
EVQLVQSGAEVKKPGASVKVSCKASGYTFTGYYMNWVRQAPGQGLEWMGEINPATGSTTYNQKFKARVTITRDTSTSTAYMELSSLRSEDTAVYYCARRDYYYGSGFYAMDYWGQGTLVTVSSGGGGSGGGGSGGGGSDIQMTQSPSSLSASVGDRVTITCQASQSVSNNLNWYQQKPGKAPKLLIYGASKLEDGVPSRFSGSGSGTDFTFTISSLQPEDFATYYCLQHRYMWTFGQGTKVEIK

SEQ ID NO: 16 - protein, 244 aa, synthetic construct
EVQLVQSGAEVKKPGASVKVSCKASGYTFTGYYMNWVRQAPGQGLEWMGEINPATGSTTYNQKFKARVTITRDTSTSTAYMELSSLRSEDTAVYYCARRDYYYGSGFYAMDYWGQGTLVTVSSGGGGSGGGGSGGGGSDIQMTQSPSSLSASVGDRVTITCQASQSVSNNLNWYQQKPGKAPKLLIYGASKLEDGVPSRFSGSGSGTDFTFTISSLQPEDFATYYCQQHRFMWTFGQGTKVEIK

SEQ ID NO: 17 - protein, 244 aa, synthetic construct
EVQLVQSGAEVKKPGASVKVSCKASGYTFTGYYMNWVRQAPGQGLEWMGEINPATGSTTYNQKFKARVTITRDTSTSTAYMELSSLRSEDTAVYYCARRDYYYGSGFYAMDYWGQGTLVTVSSGGGGSGGGGSGGGGSDIQMTQSPSSLSASVGDRVTITCQASQSVSNNLNWYQQKPGKAPKLLIYGASKLEDGVPSRFSGSGSGTDFTFTISSLQPEDFATYYCQQHRYMWTFGQGTKVEIK

SEQ ID NO: 18 - protein, 244 aa, synthetic construct
EVQLVQSGAEVKKPGASVKVSCKASGYTFTGYYMNWVRQAPGQGLEWMGEINPATGSTTYNQKFKARVTITRDTSTSTAYMELSSLRSEDTAVYYCARRDYYLGSNSYALDYWGQGTLVTVSSGGGGSGGGGSGGGGSDIQMTQSPSSLSASVGDRVTITCQASQSVSNNLNWYQQKPGKAPKLLIYGASKLEDGVPSRFSGSGSGTDFTFTISSLQPEDFATYYCLQHRYMWTFGQGTKVEIK

SEQ ID NO: 19 - protein, 244 aa, synthetic construct
EVQLVQSGAEVKKPGASVKVSCKASGYTFTGYYMNWVRQAPGQGLEWMGEINPATGSTTYNQKFKARVTITRDTSTSTAYMELSSLRSEDTAVYYCARRDYYLGSNSYALDYWGQGTLVTVSSGGGGSGGGGSGGGGSDIQMTQSPSSLSASVGDRVTITCQASQSVSNNLNWYQQKPGKAPKLLIYGASKLEDGVPSRFSGSGSGTDFTFTISSLQPEDFATYYCQQHRFMWTFGQGTKVEIK

SEQ ID NO: 20 - protein, 21 aa, synthetic construct
MALPVTALLLPLALLLHAARP

SEQ ID NO: 21 - protein, 45 aa, synthetic construct
TTTPAPRPPTPAPTIASQPLSLRPEACRPAAGGAVHTRGLDFACD

SEQ ID NO: 22 - protein, 27 aa, synthetic construct
FWVLVVVGGVLACYSLLVTVAFIIFWV

SEQ ID NO: 23 - protein, 41 aa, synthetic construct
RSKRSRLLHSDYMNMTPRRPGPTRKHYQPYAPPRDFAAYRS

SEQ ID NO: 24 - protein, 113 aa, synthetic construct
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 25 - protein, 21 aa, synthetic construct
IYIWAPLAGTCGVLLLSLVIT

SEQ ID NO: 26 - protein, 42 aa, synthetic construct
KRGRKKLLYIFKQPFMRPVQTTQEEDGCSCRFPEEEEGGCEL

SEQ ID NO: 27 - protein, 11 aa, synthetic construct
PKSCDKTHTCP

SEQ ID NO: 28 - protein, 107 aa, synthetic construct
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 12 aa, synthetic construct
ESKYGPPCPSCP

SEQ ID NO: 30 - protein, 12 aa, synthetic construct
ESKYGPPCPPCP

SEQ ID NO: 31 - protein, 209 aa, synthetic construct
MASMGLQVMGIALAVLGWLAVMLCCALPMWRVTAFIGSNIVTSQTIWEGLWMNCVVQSTGQMQCKVYDSLLALPQDLQAARALVIISIIVAALGVLLSVVGGKCTNCLEDESAKAKTMIVAGVVFLLAGLMVIVPVSWTAHNIIQDFYNPLVASGQKREMGASLYVGWAASGLLLLGGGLLCCNCPPRTDKPYSAKYSAARSAAASNYV

SEQ ID NO: 32 - protein, 220 aa, synthetic construct
MASAGMQILGVVLTLLGWVNGLVSCALPMWKVTAFIGNSIVVAQVVWEGLWMSCVVQSTGQMQCKVYDSLLALPQDLQAARALCVIALLVALFGLLVYLAGAKCTTCVEEKDSKARLVLTSGIVFVISGVLTLIPVCWTAHAIIRDFYNPLVAEAQKRELGASLYLGWAASGLLLLGGGLLCCTCPSGGSQGPSHYMARYSTSAPAISRGPSEYPTKNYV

SEQ ID NO: 33 - protein, 217 aa, synthetic construct
MASTGLELLGMTLAVLGWLGTLVSCALPLWKVTAFIGNSIVVAQVVWEGLWMSCVVQSTGQMQCKVYDSLLALPQDLQAARALCVIALLLALLGLLVAITGAQCTTCVEDEGAKARIVLTAGVILLLAGILVLIPVCWTAHAIIQDFYNPLVAEALKRELGASLYLGWAAAALLMLGGGLLCCTCPPPQVERPRGPRLGYSIPSRSGASGLDKRDYV

SEQ ID NO: 34 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 5 aa, synthetic construct
GYYMN

SEQ ID NO: 36 - protein, 17 aa, synthetic construct
EINPATGSTTYNQKFKA

SEQ ID NO: 37 - protein, 14 aa, synthetic construct
RDYYYGSGFYAMDY

SEQ ID NO: 38 - protein, 11 aa, synthetic construct
QASQSVSNNLN

SEQ ID NO: 39 - protein, 7 aa, synthetic construct
GASKLED

SEQ ID NO: 40 - protein, 8 aa, synthetic construct
LQHRYLWT

SEQ ID NO: 41 - protein, 14 aa, synthetic construct
RDYYLGSNSYALDY

SEQ ID NO: 42 - protein, 8 aa, synthetic construct
LQHRYMWT

SEQ ID NO: 43 - protein, 8 aa, synthetic construct
QQHRFMWT

SEQ ID NO: 44 - protein, 8 aa, synthetic construct
QQHRYMWT

SEQ ID NO: 45 - protein, 181 aa, synthetic construct
FWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 46 - protein, 179 aa, synthetic construct
IYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 47 - protein, 223 aa, synthetic construct
FWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 48 - protein, 470 aa, synthetic construct
EVQLVQSGAEVKKPGASVKVSCKASGYTFTGYYMNWVRQAPGQGLEWMGEINPATGSTTYNQKFKARVTITRDTSTSTAYMELSSLRSEDTAVYYCARRDYYYGSGFYAMDYWGQGTLVTVSSGGGGSGGGGSGGGGSDIQMTQSPSSLSASVGDRVTITCQASQSVSNNLNWYQQKPGKAPKLLIYGASKLEDGVPSRFSGSGSGTDFTFTISSLQPEDFATYYCLQHRYLWTFGQGTKVEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 49 - protein, 470 aa, synthetic construct
EVQLVQSGAEVKKPGASVKVSCKASGYTFTGYYMNWVRQAPGQGLEWMGEINPATGSTTYNQKFKARVTITRDTSTSTAYMELSSLRSEDTAVYYCARRDYYYGSGFYAMDYWGQGTLVTVSSGGGGSGGGGSGGGGSDIQMTQSPSSLSASVGDRVTITCQASQSVSNNLNWYQQKPGKAPKLLIYGASKLEDGVPSRFSGSGSGTDFTFTISSLQPEDFATYYCQQHRYMWTFGQGTKVEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 50 - protein, 239 aa, synthetic construct
EVQLQQSGPELVKPGASMKISCKASGYSFTGYTMNWVKQSHGKNLEWIGLINPYNGGTIYNQKFKGKATLTVDKSSSTAYMELLSLTSEDSAVYYCARDYGFVLDYWGQGTTLTVSSGGGGSGGGGSGGGGSQIVLTQSPSIMSVSPGEKVTITCSASSSVSYMHWFQQKPGTSPKLSIYSTSNLASGVPARFSGRGSGTSYSLTISRVAAEDAATYYCQQRSNYPPWTFGGGTKLEIK

SEQ ID NO: 51 - protein, 465 aa, synthetic construct
EVQLQQSGPELVKPGASMKISCKASGYSFTGYTMNWVKQSHGKNLEWIGLINPYNGGTIYNQKFKGKATLTVDKSSSTAYMELLSLTSEDSAVYYCARDYGFVLDYWGQGTTLTVSSGGGGSGGGGSGGGGSQIVLTQSPSIMSVSPGEKVTITCSASSSVSYMHWFQQKPGTSPKLSIYSTSNLASGVPARFSGRGSGTSYSLTISRVAAEDAATYYCQQRSNYPPWTFGGGTKLEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 52 - protein, 14 aa, synthetic construct
  VARIANT 5: note = X =Y or L or a conservative substitution thereof
  VARIANT 8: note = X = G or N or a conservative substitution thereof
  VARIANT 9: note = X = F or S or a conservative substitution thereof
  VARIANT 12: note = X = M or L or a conservative substitution thereof
RDYYXGSXXYAXDY

SEQ ID NO: 53 - protein, 8 aa, synthetic construct
  VARIANT 1: note = X = L or Q or a conservative substitution thereof
  VARIANT 5: note = X = Y or F or a conservative substitution thereof
  VARIANT 6: note = X = L or M or a conservative substitution thereof
XQHRXXWT

(19) United States
(12) Patent Application Publication
(10) Pub. No.: US 2026/0265368 A1
(43) Pub. Date: Sep. 10, 2026

(54) ANTIBODY AND USE THEREOF

(71) Applicant: CARSGEN LIFE SCIENCES CO., LTD, Shanghai (CN)

(72) Inventors: Liang ZHOU, Shanghai (CN); Pengju REN, Shanghai (CN); Peng WANG, Shanghai (CN); Zonghai LI, Shanghai (CN)

(21) Appl. No.: 19/124,357

(22) PCT Filed: Oct. 25, 2023
(86) PCT No.: PCT/CN2023/126593

§ 371 Date: Apr. 25, 2025

(30) Foreign Application Priority Data
Oct. 25, 2022 (CN) 202211308680.4

(51) Int. Cl. C07K 16/28; A61K 39/395; A61K 40/11; A61K 40/31; A61K 40/42; A61K 45/06; A61K 47/68; A61P 35/00; C12N 5/0783

(52) U.S. Cl. CPC C07K 16/28; A61K 39/3955; A61K 40/11; A61K 40/31; A61K 40/4202; A61K 45/06; A61K 47/6849; A61P 35/00; C12N 5/0636; C07K 2317/24; C07K 2317/565; C07K 2317/567; C07K 2317/622; C07K 2317/92; C12N 2510/00

(57) ABSTRACT

The present invention relates to an antibody that specifically binds to CLDN6, and the use thereof.

### TECHNICAL FIELD

The present invention relates to the field of tumor immunotherapy or diagnosis, and more specifically, to an antibody specifically binding to CLDN6 and uses thereof.

### SEQUENCE LISTING

The present application is being filed along with a Sequence Listing in electronic format. The Sequence Listing is provided as a file entitled Sequence Listing.xml created on Apr. 24, 2025, which is 55.9 KB in size. The information in the electronic format of the Sequence Listing is incorporated herein by reference in its entirety.

### BACKGROUND

The claudin (CLDN) gene family encodes membrane proteins that are essential components of tight junctions. CLDN protein comprises four transmembrane (TM) helices (TM1, TM2, TM3, and TM4) and two extracellular loops (EL1 and EL2), with both N- and C-termini located intracellularly. The extracellular loops of CLDN proteins from adjacent cells interact to seal cellular sheets and regulate paracellular transport between luminal and basolateral spaces. CLDN proteins play roles in various human diseases and pathologies. CLDN6, a member of the CLDN family, is not expressed in normal adult tissues but is highly expressed in multiple solid tumors, comprising ovarian, testicular, and endometrial cancers. Therefore, CLDN6 is a potential therapeutic target in the Claudin family for cancer treatment. However, the extracellular domains of CLDN6 share high sequence homology with those of its family members CLDN9 and CLDN4, thus posing significant challenges in developing antibodies specifically targeting CLDN6.

### SUMMARY OF THE INVENTION

The purpose of the present invention is to provide an antibody that specifically recognize CLDN6. The present invention also relates to a method for preparing the anti-CLDN6-specific antibody, comprising humanized antibody production techniques and phage display library screening. The present invention also relates to the study of the properties and specificity of anti-CLDN6 antibodies (comprising but not limited to scFv formats). The present invention further provides CLDN6-targeted chimeric antigen receptors (CARs) and preparation methods thereof. The present invention also provides isolated nucleic acids encoding the anti-CLDN6 antibodies and CARs of the invention. The present invention also provides host cells containing the nucleic acids of the present invention. The method comprises culturing the host cells to produce the antibody or CARs. The antibody and/or CARs of the invention are used for the tumor therapy or diagnosis.

In the first aspect, an antibody that recognizes CLDN6 or an antigen-binding fragment thereof is provided, selected from the group consisting of:
(1) The antibody or antigen-binding fragment comprises a heavy chain variable region, wherein the HCDR3 of said heavy chain variable region has at least 95%, 96%, 97%, 98%, or 99% amino acid sequence identity to the HCDR3 shown in SEQ ID NO: 41;
(2) The antibody or antigen-binding fragment comprises a heavy chain variable region, wherein said heavy chain variable region comprises HCDR1 shown as GYYMN (SEQ ID NO: 35); and/or
HCDR2 shown as EINPATGSTTYNQKFKA (SEQ ID NO: 36); and/or
HCDR3 shown as RDYYX₁GSX₂X₃YAX₄DY (SEQ ID NO: 52), wherein X₁ is Y or L or a conservatively substituted amino acid residue of Y or L; X₂ is G or N or a conservatively substituted amino acid residue of G or N; X₃ is F or S or a conservatively substituted amino acid residue of F or S; and X₄ is M or L or a conservatively substituted amino acid residue of M or L;
(3) The antibody or antigen-binding fragment comprises a heavy chain variable region, wherein the HCDR1, HCDR2, and HCDR3 of the heavy chain variable region have at least 85%, 90%, 91%, 92%, 93%, 94%, 95%, 96%, 97%, 98%, or 99% amino acid sequence identity to the HCDR1, HCDR2, and HCDR3 shown in SEQ ID NO: 35, 36, and 37, respectively;
(4) The antibody or antigen-binding fragment comprises a light chain variable region, wherein the LCDR3 of the light chain variable region has at least 60%, 65%, 70%, 75%, 80%, 85%, 90%, 91%, 92%, 93%, 94%, 95%, 96%, 97%, 98%, or 99% amino acid sequence identity to the LCDR3 shown in SEQ ID NO: 42, 43, or 44;
(5) The antibody or antigen-binding fragment comprises a light chain variable region, wherein the light chain variable region comprises LCDR1 shown as QASQSVSNNLN (SEQ ID NO: 38); and/or
LCDR2 shown as GASKLED (SEQ ID NO: 39); and/or
LCDR3 shown as X₅QHRX₆X₇WT (SEQ ID NO: 53), wherein X₅ is L or Q, or a conservatively substituted amino acid residue of L or Q; X₆ is Y or F, or a conservatively substituted amino acid residue of Y or F; and X₇ is L or M, or a conservatively substituted amino acid residue of L or M;
(6) The antibody or antigen-binding fragment comprises a light chain variable region, wherein the LCDR1, LCDR2, and LCDR3 of the light chain variable region have at least 85%, 90%, 91%, 92%, 93%, 94%, 95%, 96%, 97%, 98%, or 99% amino acid sequence identity to the LCDR1, LCDR2, and LCDR3 shown in SEQ ID NO: 38, 39, and 40, respectively;
(7) The antibody or antigen-binding fragment comprises a heavy chain variable region as described in any one of (1) to (3) and a light chain variable region as described in any one of (4) to (6);
(8) The antibody or antigen-binding fragment is a variant of the antibodies or antigen-binding fragments described in any one of (1) to (7), and the variant comprises at least one but no more than 7, 6, 5, 4, 3, or 2 amino acid changes in total on 1, 2, 3, 4, 5, or 6 CDR regions, while maintains the same or similar activity as the antibody or antigen-binding fragment as described in any one of (1) to (7).

In a preferred embodiment, HCDR2 of the antibody or antigen-binding fragment comprises the amino acid sequence set forth in SEQ ID NO: 36.

In one embodiment, the antibody or antigen-binding fragment is selected from the group consisting of:
(1) The antibody or antigen-binding fragment comprises a heavy chain variable region, wherein the heavy chain variable region comprises HCDR1 shown in SEQ ID NO: 35, and/or HCDR2 shown in SEQ ID NO: 36, and/or HCDR3 shown in SEQ ID NO: 37 or 41;
(2) The antibody or antigen-binding fragment comprises a light chain variable region, wherein the light chain variable region comprises LCDR1 shown in SEQ ID NO: 38, and/or LCDR2 shown in SEQ ID NO: 39, and/or LCDR3 shown in SEQ ID NO: 40, 42, 43, or 44;
(3) The antibody or antigen-binding fragment comprises the heavy chain variable region of (1) and the light chain variable region of (2);
(4) The antibody or antigen-binding fragment is a variant of any one of (1) to (3), wherein the variant comprises at least one but no more than 7, 6, 5, 4, 3, or 2 amino acid changes in total on 1, 2, 3, 4, 5, or 6 CDR regions, and maintains the same or similar activity as the antibody or antigen-binding fragment of any one of (1) to (3).

In one embodiment, the antibody or antigen-binding fragment is selected from the group consisting of:
(1) The antibody or antigen-binding fragment comprises HCDR1 shown in SEQ ID NO: 35, HCDR2 shown in SEQ ID NO: 36, HCDR3 shown in SEQ ID NO: 37, LCDR1 shown in SEQ ID NO: 38, LCDR2 shown in SEQ ID NO: 39, and LCDR3 shown in SEQ ID NO: 40; or
(2) The antibody or antigen-binding fragment comprises HCDR1 shown in SEQ ID NO: 35, HCDR2 shown in SEQ ID NO: 36, HCDR3 shown in SEQ ID NO: 41, LCDR1 shown in SEQ ID NO: 38, LCDR2 shown in SEQ ID NO: 39, and LCDR3 shown in SEQ ID NO: 40; or
(3) The antibody or antigen-binding fragment comprises HCDR1 shown in SEQ ID NO: 35, HCDR2 shown in SEQ ID NO: 36, HCDR3 shown in SEQ ID NO: 37, LCDR1 shown in SEQ ID NO: 38, LCDR2 shown in SEQ ID NO: 39, and LCDR3 shown in SEQ ID NO: 42; or
(4) The antibody or antigen-binding fragment comprises HCDR1 shown in SEQ ID NO: 35, HCDR2 shown in SEQ ID NO: 36, HCDR3 shown in SEQ ID NO: 37, LCDR1 shown in SEQ ID NO: 38, LCDR2 shown in SEQ ID NO: 39, and LCDR3 shown in SEQ ID NO: 43; or
(5) The antibody or antigen-binding fragment comprises HCDR1 shown in SEQ ID NO: 35, HCDR2 shown in SEQ ID NO: 36, HCDR3 shown in SEQ ID NO: 41, LCDR1 shown in SEQ ID NO: 38, LCDR2 shown in SEQ ID NO: 39, and LCDR3 shown in SEQ ID NO: 42; or
(6) The antibody or antigen-binding fragment comprises HCDR1 shown in SEQ ID NO: 35, HCDR2 shown in SEQ ID NO: 36, HCDR3 shown in SEQ ID NO: 41, LCDR1 shown in SEQ ID NO: 38, LCDR2 shown in SEQ ID NO: 39, and LCDR3 shown in SEQ ID NO: 43; or
(7) The antibody or antigen-binding fragment comprises HCDR1 shown in SEQ ID NO: 35, HCDR2 shown in SEQ ID NO: 36, HCDR3 shown in SEQ ID NO: 37, LCDR1 shown in SEQ ID NO: 38, LCDR2 shown in SEQ ID NO: 39, and LCDR3 shown in SEQ ID NO: 44; or
(8) The antibody or antigen-binding fragment is a variant of any one of (1) to (7), wherein the variant comprises at least one but no more than 7, 6, 5, 4, 3, or 2 amino acid changes in total on 1, 2, 3, 4, 5, or 6 CDR regions, and maintains the same or similar activity as the antibody or antigen-binding fragment of any one of (1) to (7).

In the second aspect, an antibody that recognizes CLDN6 or an antigen-binding fragment thereof is provided, the antibody is selected from the group consisting of:
(1) The antibody or antigen-binding fragment comprises a heavy chain variable region, wherein the heavy chain variable region comprises the amino acid sequence shown in SEQ ID NO: 1 or 5, or an amino acid sequence with at least 80%, 85%, 90%, 91%, 92%, 93%, 94%, 95%, 96%, 97%, 98%, or 99% identity thereto;
(2) The antibody or antigen-binding fragment comprises a light chain variable region, wherein the light chain variable region comprises the amino acid sequence shown in SEQ ID NO: 3, 7, 9 or 11, or an amino acid sequence with at least 80%, 85%, 90%, 91%, 92%, 93%, 94%, 95%, 96%, 97%, 98%, or 99% identity thereto;
(3) The antibody or antigen-binding fragment comprises the heavy chain variable region of (1) and the light chain variable region of (2);
(4) The antibody or antigen-binding fragment is a variant of any one of (1) to (3), wherein the variant comprises at least one but no more than 20, 15, 10, 9, 8, 7, 6, 5, 4, 3 or 2 amino acid changes on VH or VL, and maintains the same or similar activity as the antibody or antigen-binding fragment of any one of (1) to (3).

In an embodiment, the antibody or antigen-binding fragment is selected from the group consisting of:
(1) the heavy chain variable region of the antibody or antigen-binding fragment comprises the amino acid sequence shown in SEQ ID NO: 1, or an amino acid sequence with at least 80%, 85%, 90%, 91%, 92%, 93%, 94%, 95%, 96%, 97%, 98%, or 99% identity thereto; and the light chain variable region of the antibody or antigen-binding fragment comprises the amino acid sequence shown in SEQ ID NO: 3, or an amino acid sequence with at least 80%, 85%, 90%, 91%, 92%, 93%, 94%, 95%, 96%, 97%, 98%, or 99% identity thereto; or
(2) the heavy chain variable region of the antibody or antigen-binding fragment comprises the amino acid sequence shown in SEQ ID NO: 5, or an amino acid sequence with at least 80%, 85%, 90%, 91%, 92%, 93%, 94%, 95%, 96%, 97%, 98%, or 99% identity thereto; and the light chain variable region of the antibody or antigen-binding fragment comprises the amino acid sequence shown in SEQ ID NO: 3, or an amino acid sequence with at least 80%, 85%, 90%, 91%, 92%, 93%, 94%, 95%, 96%, 97%, 98%, or 99% identity thereto; or
(3) the heavy chain variable region of the antibody or antigen-binding fragment comprises the amino acid sequence shown in SEQ ID NO: 1, or an amino acid sequence with at least 80%, 85%, 90%, 91%, 92%, 93%, 94%, 95%, 96%, 97%, 98%, or 99% identity thereto; and the light chain variable region of the antibody or antigen-binding fragment comprises the amino acid sequence shown in SEQ ID NO: 7, or an amino acid sequence with at least 80%, 85%, 90%, 91%, 92%, 93%, 94%, 95%, 96%, 97%, 98%, or 99% identity thereto; or
(4) the heavy chain variable region of the antibody or antigen-binding fragment comprises the amino acid sequence shown in SEQ ID NO: 1, or an amino acid sequence with at least 80%, 85%, 90%, 91%, 92%, 93%, 94%, 95%, 96%, 97%, 98%, or 99% identity thereto; and the light chain variable region of the antibody or antigen-binding fragment comprises the amino acid sequence shown in SEQ ID NO: 9, or an amino acid sequence with at least 80%, 85%, 90%, 91%, 92%, 93%, 94%, 95%, 96%, 97%, 98%, or 99% identity thereto; or
(5) the heavy chain variable region of the antibody or antigen-binding fragment comprises the amino acid sequence shown in SEQ ID NO: 5, or an amino acid sequence with at least 80%, 85%, 90%, 91%, 92%, 93%, 94%, 95%, 96%, 97%, 98%, or 99% identity thereto; and the light chain variable region of the antibody or antigen-binding fragment comprises the amino acid sequence shown in SEQ ID NO: 7, or an amino acid sequence with at least 80%, 85%, 90%, 91%, 92%, 93%, 94%, 95%, 96%, 97%, 98%, or 99% identity thereto; or
(6) the heavy chain variable region of the antibody or antigen-binding fragment comprises the amino acid sequence shown in SEQ ID NO: 5, or an amino acid sequence with at least 80%, 85%, 90%, 91%, 92%, 93%, 94%, 95%, 96%, 97%, 98%, or 99% identity thereto; and the light chain variable region of the antibody or antigen-binding fragment comprises the amino acid sequence shown in SEQ ID NO: 9, or an amino acid sequence with at least 80%, 85%, 90%, 91%, 92%, 93%, 94%, 95%, 96%, 97%, 98%, or 99% identity thereto; or
(7) the heavy chain variable region of the antibody or antigen-binding fragment comprises the amino acid sequence shown in SEQ ID NO: 1, or an amino acid sequence with at least 80%, 85%, 90%, 91%, 92%, 93%, 94%, 95%, 96%, 97%, 98%, or 99% identity thereto; and the light chain variable region of the antibody or antigen-binding fragment comprises the amino acid sequence shown in SEQ ID NO: 11, or an amino acid sequence with at least 80%, 85%, 90%, 91%, 92%, 93%, 94%, 95%, 96%, 97%, 98%, or 99% identity thereto; or
(8) The antibody or antigen-binding fragment is a variant of any one of (1) to (7), wherein the variant comprises at least one but no more than 20, 15, 10, 9, 8, 7, 6, 5, 4, 3 or 2 amino acid changes on VH or VL, and maintains the same or similar activity as the antibody or antigen-binding fragment of any one of (1) to (7).

In the third aspect, an antibody that recognizes CLDN6 or an antigen-binding fragment thereof is provided, wherein the antibody or antigen-binding fragment comprises a heavy chain variable region and a light chain variable region, the light chain variable region comprises the amino acid sequence shown in SEQ ID NO: 3, 7, 9 or 11, or an amino acid sequence with at least 80%, 85%, 90%, 91%, 92%, 93%, 94%, 95%, 96%, 97%, 98%, or 99% identity thereto; and
the heavy chain variable region comprises HCDR1 shown in SEQ ID NO: 35, HCDR2 shown in SEQ ID NO: 36, and HCDR3 shown in SEQ ID NO: 37 or 41.

In an embodiment, the heavy chain variable region of the antibody or antigen-binding fragment comprises the amino acid sequence shown in SEQ ID NO: 1 or 5, or an amino acid sequence with at least 80%, 85%, 90%, 91%, 92%, 93%, 94%, 95%, 96%, 97%, 98%, or 99% identity thereto.

In the fourth aspect, an antibody that recognizes CLDN6 or an antigen-binding fragment thereof is provided, wherein the antibody or antigen-binding fragment comprises a heavy chain variable region and a light chain variable region, the heavy chain variable region comprises the amino acid sequence shown in SEQ ID NO: 1 or 5, or an amino acid sequence with at least 80%, 85%, 90%, 91%, 92%, 93%, 94%, 95%, 96%, 97%, 98%, or 99% identity thereto; and
the light chain variable region comprises LCDR1 shown in SEQ ID NO: 38, LCDR2 shown in SEQ ID NO: 39, and LCDR3 shown in SEQ ID NO: 40, 42, 43 or 44.

In an embodiment, the light chain variable region of the antibody or antigen-binding fragment comprises the amino acid sequence shown in SEQ ID NO: 3, 7, 9 or 11, or an amino acid sequence with at least 80%, 85%, 90%, 91%, 92%, 93%, 94%, 95%, 96%, 97%, 98%, or 99% identity thereto.

In one embodiment, the antibody or antigen-binding fragment described in any one of the preceding items is selected from a full-length antibody, scFv, single-domain antibody, Fab fragment, Fab′ fragment, Fv fragment, F(ab′) 2 fragment, Fd fragment, dAb fragment, multifunctional antibody, IgG4 antibody, scFv-Fc antibody, hybridoma antibody, chimeric antibody, humanized antibody, fully human antibody, or monoclonal antibody.

In one embodiment, the antibody or antigen-binding fragment comprises an amino acid sequence shown in SEQ ID NO: 13, 14, 15, 16, 17, 18, or 19, or an amino acid sequence having at least 80%, 85%, 90%, 91%, 92%, 93%, 94%, 95%, 96%, 97%, 98%, or 99% identity thereto, or comprises at least one but no more than 20, 15, 10, 9, 8, 7, 6, 5, 4, 3, or 2 amino acid changes in the sequences as said above.

In one embodiment, the antibody or antigen-binding fragment binds to CLDN6 without significantly binding to CLDN4 or CLDN9; and/or, the antibody or antigen-binding fragment binds to cells expressing CLDN6 without significantly binding to cells expressing CLDN4, CLDN9, or a combination thereof.

In the fifth aspect, an immunoconjugate is provided comprising: the antibody or antigen-binding fragment according to any one of the first to fourth aspects, and a functional molecule linked thereto.

In one embodiment, the functional molecule is selected from: a molecule targeting tumor surface markers, a tumor-inhibiting molecule, a molecule targeting surface markers of immune cells, or a detectable label.

In one embodiment, the molecule targeting tumor surface markers is an antibody or ligand binding to tumor surface markers other than CLDN6.

In one embodiment, the tumor-inhibiting molecule is an anti-tumor cytokine or an anti-tumor toxin.

In one embodiment, the cytokine is selected from: IL-7, IL-12, IL-15, IL-18, IL-21, type I interferon, or TNF-α.

In one embodiment, the molecule targeting surface markers of immune cells is an antibody binding to T cell surface markers, which forms a T cell-engaging bispecific antibody with the antibody or antigen-binding fragment described in any one of the first to fourth aspects.

In one embodiment, the surface markers targeting immune cells are selected from: CD3, CD16, or CD28.

In one embodiment, the antibody targeting surface markers of immune cells is an anti-CD3 antibody.

In one embodiment, the immunoconjugate further comprises a linker peptide between the antibody or antigen-binding fragment described in any one of the first to fourth aspects and the functional molecule connected thereto.

In the 6^{th} aspect, a chimeric receptor comprising an extracellular region is provided, wherein the extracellular region comprises the antibody or antigen-binding fragment according to any one of the 1st-4th aspects;

the chimeric receptor comprises: a chimeric antigen receptor (CAR), a chimeric T-cell receptor, a T-cell antigen coupler (TAC), a synthetic polypeptide receptor (synNotch), or a combination thereof.

In one embodiment, the chimeric receptor is a chimeric antigen receptor (CAR), which comprises the antibody or antigen-binding fragment according to any one of the 1st-4th aspects, a transmembrane region, and an intracellular signaling region.

In one embodiment, the antibody or antigen-binding fragment is linked to the transmembrane region via a hinge domain.

In one embodiment, the transmembrane region of the chimeric receptor comprises a transmembrane region selected from that of TCR α, β, γ, or ζ chains, CD3ε, CD3δ, CD45, CD4, CD5, CD8, CD9, CD16, CD22, CD27, CD28, CD33, CD37, CD45, CD64, CD80, CD134, CD137, CD152, CD154, or PD1.

In one embodiment, the transmembrane region is selected from the transmembrane domains of CD8 or CD28.

In one embodiment, the transmembrane region is selected from the sequences shown in SEQ ID NO: 25 or 22, or an amino acid sequence having at least 80%, 85%, 90%, 95%, 96%, 97%, 98%, or 99% sequence identity thereto.

In one embodiment, the intracellular signaling region of the chimeric receptor comprises a primary signaling domain.

In one embodiment, the primary signaling domain is selected from TCRξ, FcRγ, FcRβ, CD3γ, CD3δ, CD3ε, CD5, CD22, CD79a, CD79b, CD278 (also referred to as “ICOS”), CD66d, or CD3ζ.

In one embodiment, the intracellular signaling region of the chimeric receptor further comprises one or more costimulatory signaling domains.

In one embodiment, the costimulatory signaling domain is selected from the intracellular signaling regions of CARD11, CD2, CD5, CD7, CD27, CD28, CD30, CD40, CD54, CD83, OX40, CD137, CD134, CD150, CD152, CD223, CD270, PD-L2, PD-L1, CD278, DAP10, LAT, NKD2C, SLP76, TRIM, FcεRIγ, MyD88, ICAM-1, LFA-1 (CD11a/CD18), 4-1BB, or 4-1BBL, or combinations thereof.

In one embodiment, the costimulatory signaling domain is selected from the intracellular signaling domains of CD28 and/or CD137.

In one embodiment, the intracellular signaling region of the chimeric receptor is selected from SEQ ID NO: 24, or the sequence shown in SEQ ID NO: 23 and 24, or the sequence shown in SEQ ID NO: 26 and 24, or the sequence shown in SEQ ID NO: 23, 26, and 24, or an amino acid sequence having at least 80%, 85%, 90%, 95%, 96%, 97%, 98%, or 99% sequence identity to the sequences as said above.

In one embodiment, the hinge region of the chimeric receptor is derived from CD8, IgG4, or IgG1.

In one embodiment, the hinge region comprises the sequence shown in SEQ ID NO: 21, 27, 28, 29, or 30, or an amino acid sequence having at least 80%, 85%, 90%, 95%, 96%, 97%, 98%, or 99% sequence identity to the sequences as said above.

In one embodiment, the chimeric receptor comprises:
An antibody or antigen-binding fragment according to any one of the first to fourth aspect, a transmembrane region of CD8/CD28, and CD3ζ; or
An antibody or antigen-binding fragment according to any one of the first to fourth aspects, a transmembrane region of CD8/CD28, an intracellular signaling region of CD137, and CD3ζ; or
An antibody or antigen-binding fragment according to any one of the first to fourth aspects, a transmembrane region of CD8/CD28, an intracellular signaling region of CD28, and CD3ζ; or
An antibody or antigen-binding fragment according to any one of the first to fourth aspects, a transmembrane region of CD8/CD28, an intracellular signaling region of CD28, CD137, and CD3ζ.

In one embodiment, the chimeric receptor comprises an amino acid sequence formed by linking any one of SEQ ID NOs: 13, 14, 15, 16, 17, 18, or 19 with any one of SEQ ID NOs: 45, 46, or 47 respectively, or an amino acid sequence having at least 80%, 85%, 90%, 95%, 96%, 97%, 98%, or 99% sequence identity to the sequences as said above.

In one embodiment, the chimeric receptor comprises the sequence shown in SEQ ID NO: 48 or 49, or an amino acid sequence having at least 80%, 85%, 90%, 95%, 96%, 97%, 98%, or 99% sequence identity to the sequences as said above.

In the 7^{th} aspect, a nucleic acid is provided, encoding the antibody or antigen-binding fragment described in any one of the first to fourth aspects, the immunoconjugate described in the fifth aspect, or the chimeric receptor described in the sixth aspect.

In the 8^{th} aspect, a vector is provided, comprising the nucleic acid described in the seventh aspect.

In the 9^{th} aspect, a cell is provided, comprising the antibody or antigen-binding fragment described in the first to fourth aspects, the immunoconjugate described in the fifth aspect, the chimeric receptor described in the sixth aspect, the nucleic acid described in the seventh aspect, and/or the vector described in the eighth aspect.

In one embodiment, the cells comprise T cells, natural killer cells, natural killer T cells, NK92 cells, cytotoxic T cells, dendritic cells, macrophages, cytokine-induced killer (CIK) cells, pluripotent stem cells, stem cell-derived immune cells, or combinations thereof.

In one embodiment, the T cells comprise natural T cells and/or T cells induced from pluripotent stem cells.

In one embodiment, the T cells comprise autologous T cells and/or allogeneic T cells.

In one embodiment, the T cells are primary T cells.

In one embodiment, the T cells are derived from human autologous T cells.

In one embodiment, the cells bind to cells expressing CLDN6 and do not significantly bind to cells expressing CLDN4, CLDN9, or combinations thereof.

In one embodiment, the cells further carry encoding sequences for exogenous cytokines; and/or further express a chimeric receptor not targeting CLDN6; and/or further express chemokines; and/or further express chemokine receptors; and/or further express a safety switch; and/or further express inhibitory molecules.

In one embodiment, the cells further carry encoding sequences for exogenous cytokines that comprise IL-7, IL-12, IL-15, IL-18, IL-21, type I interferon, or TNF-α.

In one embodiment, the cells further express chemokines, wherein the chemokines comprise CCL19 or CCL21.

In one embodiment, the cells further express chemokine receptors, wherein the chemokine receptors comprise CCR2, CCR4, CCR5, CXCR2, CXCR4, or CXCR5.

In one embodiment, the cells further express a safety switch, wherein the safety switch comprises iCaspase-9, truncated EGFR, or RQR8.

In one embodiment, the cells further express inhibitory molecules, wherein the inhibitory molecules comprise siRNA that reduces PD-1 expression or a protein that blocks PD-L1.

In the tenth aspect, a pharmaceutical composition is provided, comprising the antibody or antigen-binding fragment according to any one of the first to fourth aspects, the immunoconjugate according to the fifth aspect, the chimeric receptor according to the sixth aspect, the nucleic acid according to the seventh aspect, the vector according to the eighth aspect, and/or the cell according to the ninth aspect, and a pharmaceutically acceptable adjuvant.

In the eleventh aspect, a combined administration is provided, comprising co-administration of the antibody or antigen-binding fragment according to any one of the first to fourth aspects; the immunoconjugate according to the fifth aspect; the chimeric receptor according to the sixth aspect; the cell according to the ninth aspect; or the pharmaceutical composition according to the tenth aspect with an agent that enhances their function.

In one embodiment, the combined administration comprises: co-administration with a chemotherapeutic agent; and/or co-administration with an agent that mitigates one or more related side effects; and/or co-administration with cells expressing a chimeric antigen receptor targeting a molecule other than CLDN6; and/or co-administration with an agent for treating diseases associated with CLDN6 expression.

In one embodiment, the agent comprises an antibody or antigen-binding fragment, cells, RNA, a vaccine, an oncolytic virus, a checkpoint inhibitor, a BTK inhibitor, a chemical medicament, a radiotherapy agent, a hormonal therapy agent, a toxin, an immunotherapeutic agent, or a combination thereof.

In the twelfth aspect, a method is provided for preparing the antibody or antigen-binding fragment according to any one of the first to fourth aspects, the immunoconjugate according to the fifth aspect, or the chimeric receptor according to the sixth aspect, wherein the method comprises culturing the cells according to the ninth aspect and isolating the antibody or antigen-binding fragment, immunoconjugate, or chimeric receptor expressed by the cells.

In the thirteenth aspect, a kit is provided, comprising the antibody or antigen-binding fragment according to any one of the first to fourth aspects, the immunoconjugate according to the fifth aspect, the chimeric receptor according to the sixth aspect, the nucleic acid according to the seventh aspect, the vector according to the eighth aspect, the cells according to the ninth aspect, and/or the pharmaceutical composition according to the tenth aspect.

In the fourteenth aspect, a use of the antibody or antigen-binding fragment according to any one of the first to fourth aspects, the immunoconjugate according to the fifth aspect, the cells according to the ninth aspect, the pharmaceutical composition according to the tenth aspect, and/or the kit according to the thirteenth aspect is provided, for:
(1) killing cells expressing CLDN6; (2) inhibiting the proliferation of cells expressing CLDN6; (3) mediating the alleviation of diseases or tumors; (4) preventing tumor formation or reformation; (5) inhibiting the metastasis of cells expressing CLDN6; (6) preparing a medicament for the treatment/diagnosis of diseases.

In one embodiment, the disease expresses CLDN6.

In one embodiment, the disease is selected from inflammatory disorders, infections, autoimmune diseases, or tumors.

In one embodiment, the tumor is a solid tumor.

In one embodiment, the tumor is ovarian cancer, breast cancer, cervical cancer, gastric cancer, lung cancer, testicular cancer, germ cell and embryonic tumors, ovarian epithelial cancer, non-small cell lung cancer, non-squamous non-small cell lung cancer, endometrial cancer, or a combination thereof.

In the fifteenth aspect, the present invention provides a pharmaceutical agent comprising the antibody or antigen-binding fragment described in the first aspect, the immunoconjugate described in the second aspect, the cells described in the ninth aspect, or the pharmaceutical composition described in the tenth aspect, for:
(1) killing cells expressing CLDN6;
(2) inhibiting the proliferation of cells expressing CLDN6;
(3) mediating the alleviation of diseases or tumors;
(4) preventing tumor formation or reformation;
(5) inhibiting the metastasis of cells expressing CLDN6;
(6) the treatment/diagnosis of diseases.

In the sixteenth aspect, a method for treating/diagnosing a disease is provided, comprising administering to a subject in need thereof an effective amount of the antibody or antigen-binding fragment as described in any one of the first to fourth aspects, the immunoconjugate as described in the fifth aspect, the cell as described in the ninth aspect, the pharmaceutical composition as described in the tenth aspect, or the kit as described in the thirteenth aspect.

In one embodiment, the disease is selected from inflammatory disorders, infections, autoimmune diseases, or tumors.

In one embodiment, the subject is a human.

In one embodiment, the cell is an autologous or allogeneic T cell for the subject.

In the 17^{th} aspect, the antibody or antigen-binding fragment as described in any one of the 1^{st}-4^{th} aspects, the immunoconjugate as described in the 5^{th} aspect, the cell as described in the 9th aspect, the pharmaceutical composition as described in the 10^{th} aspect, and/or the kit as described in the 13^{th} aspect, for use in the treatment/diagnosis of a disease expressing CLDN6.

In one embodiment, the disease is selected from inflammatory disorders, infections, autoimmune diseases, or tumors.

In one embodiment, the tumor is a solid tumor.

In one embodiment, the tumor is ovarian cancer, breast cancer, cervical cancer, gastric cancer, lung cancer, testicular cancer, germ cell and embryonic tumors, ovarian epithelial carcinoma, non-small cell lung cancer, non-squamous non-small cell lung cancer, endometrial cancer, or a combination thereof.

It should be understood that, within the scope of the present invention, the above technical features of the present invention and the technical features specifically described in the following (such as in the Examples) can be combined with each other to form new or preferred technical solutions. Such technical solutions will not be described one by one due to the limited contents.

### DESCRIPTION OF DRAWINGS

FIG. 1 shows flow cytometry results of stable transfected cell lines 293T-CLDN4, 293T-CLDN6, and 293T-CLDN9.

FIG. 2 shows the specific binding of antibody H1 (scFv-huFc) to 293T-CLDN6 cells.

FIG. 3 shows the EC50 of antibody H1 binding to 293T-CLDN6 cells.

FIG. 4 shows that all antibodies P1, P2, P3, and P4 specifically bind to 293T-CLDN6 cells.

FIG. 5 shows the EC50 of antibodies P1, P2, P3, and P4 binding to 293T-CLDN6 cells.

FIG. 6 shows that antibodies H1, M1, and M2 specifically bind to 293T-CLDN6 cells.

FIG. 7 shows the EC50s of antibodies M1 and M2 binding to 293T-CLDN6 cells.

FIG. 8 shows the positivity rates of H1-28Z CAR T cells and P4-28Z CAR T cells.

FIG. 9 shows the in vitro specific killing of H1-28Z CAR T cells and P4-28Z CAR T cells against target cells;

FIG. 10 shows the in vitro killing of H1-28Z CAR T cells and P4-28Z CAR T cells against target cells.

FIGS. 11A, 11B, 11C and 11D show the antitumor effects of H1-28Z CAR T cells and P4-28Z CAR T cells on subcutaneous xenograft tumors in NPG mice bearing human ovarian cancer cells (FIG. 11A), changes in mouse body weight (FIG. 11B), tumor weight changes (FIG. 11C), and the survival of human T cells in peripheral blood of mice (FIG. 11D).

### MODES FOR CARRYING OUT THE INVENTION

In the present invention, humanized antibodies that specifically recognize CLDN6 (comprising fragments thereof, such as the heavy chain variable region (VH), light chain variable region (VL), and scFv), chimeric receptors comprising these antibodies, nucleic acids encoding these antibodies, and cells expressing these antibodies are provided. The antibodies of the invention can be used to prepare targeted anti-tumor medicament and diagnostic agents for tumors. In the invention, methods for preparing and using these antibodies and cells expressing them are also provided.

### Terms

Unless specifically defined, all technical and scientific terms used herein have the same meanings as commonly understood by a skilled person in the fields of gene therapy, biochemistry, genetics, and molecular biology. All methods and materials similar or equivalent to those described herein may be used in the practice or testing of the present invention, wherein the methods and materials described herein are deemed suitable. All publications, patent applications, patents, and other references cited herein are incorporated by reference in their entirety. In case of conflict, the present specification, comprising definitions, shall prevail. Furthermore, unless otherwise specified, the materials, methods, and examples are illustrative only and not intended to be limiting. Based on the disclosure of the present invention, a skilled person will appreciate that many variations or modifications may be made to the specific embodiments disclosed herein while still achieving the same or similar results without departing from the spirit and scope of the invention. The scope of the invention is not limited to the specific embodiments described herein (which are merely illustrative of various aspects of the invention), and functionally equivalent methods and components will fall within the scope of the invention. The invention encompasses variations and modifications of the subject matter described herein for various uses and conditions.

Unless otherwise specified, the practice of the present invention will employ conventional techniques of cell biology, cell culture, molecular biology, transgenic biology, microbiology, recombinant DNA, and immunology, which fall within the skill of the art. Such techniques are fully explained in the literatures.

In this disclosure, all aspects of the claimed subject matter are presented in the form of ranges. It should be understood that the description in a range form is merely for convenience and brevity and should not be construed as a rigid limitation on the scope of the claimed subject matter. Therefore, the description of a range should be considered to have specifically disclosed all possible sub-ranges as well as individual numerical values within the stated range. For example, when a range of values is provided, it should be understood that every intermediate value between the upper and lower limits of the range, as well as any other stated or intermediate values within the range, are comprised in the claimed subject matter. The upper and lower limits of the range also fall within the scope of the claimed subject matter. Smaller ranges may independently comprise the upper and lower limits of these smaller ranges, which also belong to the scope of the claimed subject matter, unless the upper and lower limits of the range are explicitly excluded. When a range comprises one or both limits, the claimed subject matter also comprises ranges that exclude either or both of said limits. This applies regardless of the breadth of the range.

The term “about” refers to the commonly accepted margin of error for various values as understood by a skilled person. When an “about” value or parameter is mentioned herein, it comprises embodiments that directly correspond to that value or parameter. For example, a description referring to “about X” encompasses “X” itself. The term “about” or “comprising” may indicate a range of ±1%, ±2%, ±3%, ±4%, ±5%, ±6%, ±7%, ±8%, ±9%, ±10%, ±11%, ±12%, ±13%, ±14%, ±15%, ±16%, ±17%, ±18%, ±19%, ±20%, ±25%, or ±30% of the stated value. Alternatively, particularly in the context of biological systems or methods, the term may refer to a range within one order of magnitude of the value, for example, within about 5 times or within about 2 times the value.

Unless otherwise indicated, any concentration ranges, percentage ranges, ratio ranges or integer ranges described herein shall be understood to comprise all integers within the stated ranges and, where appropriate, fractional values thereof (e.g. tenths or hundredths of an integer).

To facilitate understanding of the present invention, certain terms are defined.

The term “Claudin 6 (CLDN6)” refers to a member of the Claudin (CLDN) family. The gene encoding human CLDN6 protein is located on the p-arm of chromosome 16 at 16p13.3 and is conserved in chimpanzees, rhesus monkeys, dogs, cattle, mice, rats, zebrafish, and frogs. The CLDN6 polypeptide may comprise an amino acid sequence or a fragment thereof having at least approximately 80%, 85%, 90%, 95%, 96%, 97%, 98%, 99%, or 100% homology or identity with the amino acid sequence encoded by transcripts expressed by the gene (NCBI GenBank Gene ID: 9074), and/or it may optionally comprise no more than one, two, or three conservative amino acid substitutions. As an example, the full-length amino acid sequence of human CLDN6 is shown in SEQ ID NO: 32.

The term “Claudin 9 (CLDN9)” refers to the most closely related family member of CLDN6. The gene encoding human CLDN9 protein is located on human chromosome 16 at 16p13.3 and comprises a single exon spanning approximately 2.1 kbp. As an example, the full-length amino acid sequence of human CLDN9 is shown as SEQ ID NO: 33.

The term “Claudin 4 (CLDN4)” also refers to a member of the CLDN family. The gene encoding human CLDN4 protein spans approximately 1.82 kbp at the chromosomal locus 17q11.23. As an example, the full-length amino acid sequence of human CLDN4 is shown as SEQ ID NO: 31.

The terms “polypeptide”, “peptide”, “protein”, and “proteins” can be used interchangeably to refer to polymers of any length comprising amino acid residues covalently linked by peptide bonds. A protein or peptide will necessarily contain at least two amino acids, with no upper limit on the number of amino acids. Polypeptides encompass any peptide or protein comprising two or more amino acids linked each other by peptide bonds. This term refers to both short chains (commonly referred to in the art as peptides, oligopeptides, or oligomers) and longer chains (typically referred to in the art as proteins). There are numerous types of proteins. Polypeptides comprise, for example, bioactive fragments, substantially homologous polypeptides, oligopeptides, homodimers, heterodimers, polypeptide variants, modified polypeptides, derivatives, analogs, fusion proteins, etc. Polypeptides comprise natural peptides, recombinant peptides, or combinations thereof. The polymer can be linear, cyclic, or branched and may comprise modified amino acids (especially conservatively modified ones) or be interrupted by non-amino acids. The term also encompasses modified amino acid polymers, such as those modified via sulfation, glycosylation, lipidation, acetylation, phosphorylation, iodination, methylation, oxidation, proteolytic processing, prenylation, racemization, selenoylation, tRNA-mediated amino acid addition (e.g., arginylation), ubiquitination, or other modifications like conjugation with labeling groups. As used herein, the term “amino acid” refers to natural and/or unnatural or synthetic amino acids, comprising glycine and D or L optical isomers, as well as amino acid analogs and peptidomimetics. A polypeptide or amino acid sequence “derived from” a specified protein refers to the origin of the polypeptide. This term also comprises a polypeptide encoded by a designated nucleic acid sequence.

The term “antibody” refers to a protein or polypeptide sequence derived from immunoglobulin molecules that specifically binds to antigens. It is used in the broadest sense in this context and comprises various antibody structures, comprising but not limited to monoclonal antibodies, polyclonal antibodies, multispecific antibodies (e.g., bispecific antibodies), antibody fragments, multichain or single-chain or intact immunoglobulins, which may originate from natural sources or recombinant sources, provided they exhibit required antigen-binding activities.

The term “Antigen-Binding Fragment” refers to an antibody or immunologically active portion thereof, i.e., a molecule containing an antigen-binding site that specifically interacts with an antigen (immunologically reactive with the antigen).

The humanized antibodies H1, P1, P2, P3, P4, M1, and M2 of the present invention can specifically bind to CLDN6 but do not bind to CLDN4 or CLDN9. In specific embodiments, the antigen-binding specificities of the antibodies H1, P1, P2, P3, P4, M1, and M2 of the present invention are superior to that of the hybridoma antibody SC27.105. Furthermore, the antigen-binding specificities of the antibodies H1, P1, P2, P3, P4, M1, and M2 of the present invention are superior to that of the control antibody C46-S(from WO2015150327A1).

Terms “specific binding”, “specific recognition”, or “specificity for . . . ” refer to measurable and reproducible interactions, such as the binding between a target and an antibody. Such binding is a determining factor for the presence of the target in the presence of other molecules. For example, an antibody specifically binding to a target exhibits greater affinity, easier binding, and/or longer binding duration to that target compared with the binding with other targets. In some embodiments, the antibody or ligand recognizes and binds to its associated binding partner protein present in a sample but essentially does not recognize or bind to other molecules in the sample. In some embodiments, the specific binding may comprise, but does not require, exclusive binding. “Multispecificity” refers to an antibody having binding specificities for at least two different sites on an antigen.

“Antibody fragment” refers to at least a portion of a full-length antibody or a recombinant variant thereof, and may refer to the antigen-binding domain of the intact antibody, such as the antigenicity-determining variable region, which is sufficient to confer recognition and specific binding of the antibody fragment to a target (e.g., an antigen). Examples of antibody fragments comprise but are not limited to: (i) Fab fragments comprising VL, VH, CL and CH1 domains, comprising Fab′ and Fab′-SH; (ii) Fd fragments comprising VH and CH1 domains; (iii) Fv fragments comprising VL and VH domains from a single antibody; (iv) dAb fragments comprising a single variable domain; (v) F(ab′) 2 fragments, bivalent fragments comprising two linked Fab fragments; (vi) antigen-binding sites of single-chain Fv (scFv) molecules; (vii) Bispecific single-chain Fv dimers; (viii) “Diabodies” or “Triabodies”, multivalent/multispecific fragments constructed via genetic fusion; (ix) scFv genetically fused to identical or different antibodies; (x) Linear antibodies; (xi) Camelid VHH domains; and (xii) Single-domain antibodies like sdAb (VH or VL). Antibody fragments can be prepared using various techniques, comprising proteolytic digestion of intact antibodies and production via recombinant host cells.

The term “scFv” refers to a fusion protein comprising at least one antibody fragment with a light-chain variable region (VL) and at least one antibody fragment with a heavy-chain variable region (VH), where the VL and VH are connected by a short flexible peptide linker. scFv can be expressed as a single-chain polypeptide while retaining the antigen-binding specificity of the parent antibody. In this context, the scFv can adopt either VL-linker-VH or VH-linker-VL orientation (e.g., relative to the N- and C-termini of the polypeptide). The linker may comprise any amino acid sequence of any length.

The term “linker” or “flexible polypeptide linker” refers to a peptide linker comprising amino acid residues (e.g., glycine and/or serine), used individually or in combination, to link the variable regions of heavy and light chains. In one embodiment, the flexible polypeptide linker is a Gly/Ser linker, comprising an amino acid sequence (Gly-Gly-Gly-Gly-Ser)ₙ, where n is an integer ≥1 (e.g., n=1, n=2, n=3, n=4, n=5, n=6, n=7, n=8, n=9, n=10). In one embodiment, the flexible polypeptide linker comprises but is not limited to (Gly₄Ser)₄ or (Gly₄Ser)₃. In one embodiment, the linker may comprise multiple repeats of (Gly₂Ser), (GlySer), or (Gly₃Ser). In one embodiment, the linker may comprise a charged residue (e.g., lysine/glutamate) to enhance solubility. In one embodiment, the linker comprises one or more prolines.

The term “antibody heavy chain” refers to the larger one of the two types of polypeptide chains in the natural conformation of an antibody molecule, which typically determines the antibody's class.

The term “antibody light chain” refers to the smaller one of the two types of polypeptide chains in the natural conformation of an antibody molecule. The κ and λ light chains refer to two major isotypes of antibody light chains.

The “classification” of antibodies refers to the type of constant domain or constant region of the heavy chains. There are five main classes of antibodies: IgA, IgD, IgE, IgG, and IgM. Some of these can be further divided into subclasses (allotypes), such as IgG1, IgG2, IgG3, IgG4, IgA1, and IgA2. The heavy chain constant domains corresponding to different types of immunoglobulins are designated as α, δ, ε, γ, and μ, respectively.

The term “variable region or variable domain” refers to the domains of the antibody heavy or light chains involved in antigen binding. The variable domains of the heavy and light chains (VH and VL, respectively) in natural antibodies typically share a similar structure, with each domain comprising four conserved framework regions (FR) and three CDRs. A single VH or VL domain may suffice to confer antigen-binding specificity. Moreover, antibodies binding to a specific antigen can be isolated by screening libraries of complementary VL or VH domains using the VH or VL domains derived from the antigen-binding antibody, respectively.

The term “hypervariable region”, “complementarity-determining region (CDR)” or “CDR” refers to regions within the variable domain of an antibody that exhibit sequence hypervariation and/or form structurally defined loops (“hypervariable loops”) and/or contain residues that contact antigens (“antigen contacts site”). These are discontinuous amino acid sequences within the antibody's variable region, which confer specificity and/or binding affinity to the antibody. In certain embodiments, CDRs can be identified using numbering systems such as Kabat, Chothia, IMGT, Gelfand, Aho, or AbM. For example, the Kabat numbering system may be used. For example, an antibody typically comprises six CDRs: three in the variable heavy chain (VH) region (HCDR1, HCDR2, HCDR3) and three in the variable light chain (VL) region (LCDR1, LCDR2, LCDR3).

The term “Fc region” or “Fc” is used to define the C-terminal region of an immunoglobulin heavy chain that comprises at least a portion of the constant region. This term encompasses both native sequence Fc regions and variant Fc regions.

“Framework region (FR)” refers to the residues in the variable domain that differ from the hypervariable region (CDR) residues. The FR of a variable domain typically comprise four FR domains: FR1, FR2, FR3, and FR4. Therefore, in the VH (or VL) domain, the CDR and FR sequences generally appear in the following order: FR1-HCDR1 (LCDR1)-FR2-HCDR2 (LCDR2)-FR3-HCDR3 (LCDR3)-FR4.

Unless otherwise specified, CDR residues and other residues in the variable domain (e.g., FR residues) are numbered according to the Kabat numbering system in this document.

The term “natural antibodies” refers to naturally occurring immunoglobulin molecules with diverse structures. For example, a natural IgG antibody is a heterotetrameric glycoprotein of approximately 150,000 Daltons, comprising two identical light chains and two identical heavy chains linked by disulfide bonds. From the N-terminus to the C-terminus, each heavy chain comprises a variable region (VH), also termed the variable heavy chain domain or heavy chain variable domain, followed by three constant domains (CH1, CH2, and CH3). Similarly, from the N-terminus to the C-terminus, each light chain comprises a variable region (VL), known as the variable light chain domain or light chain variable domain, followed by light chain constant (CL) domains. Based on the amino acid sequence of the constant domains, antibody light chains can be classified into two types: κ (kappa) or λ (lambda).

The terms “full-length antibody”, “full antibody”, “complete antibody” and “intact antibody” are used interchangeably. They refer to full-length antibodies that have a structure essentially similar to natural antibodies, or comprise heavy chains with an Fc region as defined herein, or comprise antigen-binding domains.

The term “Single domain antibody (sdAb)” refers to a type of antibody that lacks the light chain and only comprises the variable region of the heavy chain, which is, due to its small molecular weight, is also known as a “Nanobody”.

The term “single-domain antibody” refers to an antibody fragment comprising either the entire or partial heavy chain variable domain or the entire or partial light chain variable domain of an antibody. In certain embodiments, the single-domain antibody is a human single-domain antibody.

The terms “monoclonal antibody” and “mAb” refer to antibodies derived from a substantially homogeneous population of antibodies, meaning that the individual antibodies within the population are identical and/or bind to the same epitope, except for possible variant antibodies (e.g., those containing naturally occurring mutations or generated during monoclonal antibody production, which typically exist in minor quantities). In contrast to polyclonal antibody preparations (which generally comprise different antibodies targeting different determinants (epitopes)), each monoclonal antibody in a monoclonal antibody preparation targets a single determinant on the antigen. Therefore, the attribute “monoclonal” means that the antibody is obtained from a substantially homogeneous population and does not imply restriction to any specific production method. For example, they can be produced via various techniques, comprising but not limited to hybridoma technology, recombinant DNA methods, phage display, and a method using transgenic animals carrying all or part of the human immunoglobulin gene locus.

The term “chimeric antibody” refers to an antibody in which part of the heavy chain and/or light chain is derived from a specific source or species, while the remaining part of the heavy and/or light chain is derived from a different source or species. In some embodiments, a chimeric antibody comprises a non-human variable region (e.g., derived from mice, rats, hamsters, rabbits, or non-human primates such as monkeys) and a human constant region. In other embodiments, the chimeric antibody is a “class-switched” antibody, where the isotype or subclass has been altered from that of the parental antibody. Chimeric antibodies also comprise antigen-binding fragments thereof. In certain embodiments, the chimeric antibody is a “humanized antibody”.

The term “humanization” refers to non-human antibodies (e.g., from rodents or primates) that are hybrid immunoglobulins, immunoglobulin chains, or fragments thereof comprising minimal sequences derived from non-human immunoglobulins. A “humanized antibody” is a chimeric antibody comprising amino acid residues from non-human CDRs and amino acid residues from human FRs. In certain embodiments, humanized antibodies will comprise substantially all of at least one (typically two) variable domain, where all or nearly all CDRs correspond to those of non-human antibodies, while all or nearly all FRs correspond to human antibody FRs. Optionally, humanized antibodies may comprise at least a portion of the constant region derived from human antibodies. Various techniques are well-known in the art for humanizing antibodies or antibody fragments, primarily involving the replacement of human antibody sequences with rodent CDRs or CDR sequences, a process known as CDR grafting. Humanized antibodies are essentially human antibodies in which some CDR residues and potentially certain FR residues are substituted with residues from analogous sites in rodent antibodies

In some embodiments, “humanized antibody” may comprise mutations, such as those introduced by random or site-directed mutagenesis in vitro or by somatic mutation in vivo. In some embodiments, the human domain for preparing a humanized antibody can be selected to reduce antigenicity. According to the so-called “best-fit” method, the variable domain sequences of rodent antibodies are screened against the complete library of known human variable domain sequences. Then, the sequence of rodents can be accepted as human FR for humanized antibody. In some embodiments, specific FRs derived from a consensus sequence are used, wherein the consensus sequence is the consensus sequence of all human antibodies having a specific subgroup of light or heavy chains. The same FR can be used for several different humanized antibodies. In some embodiments, some FR residues of a humanized antibody are replaced by corresponding residues from non-human antibodies (e.g., antibodies from which CDR residues are derived), for example, to restore or improve antibody specificity or affinity. Or, an antibody can be obtained by transplanting the amino acid sequences of CDRs of VH and VL of non-human animal antibodies to the corresponding CDRs of VH and VL of human antibodies. The region other than the CDR of VH and VL is called frame region (hereinafter referred to as FR). In an example, a cDNA encoding the amino acid sequence of VH composed of CDR of nonhuman animal antibody and FR of VH of arbitrary human antibody, and a cDNA encoding the amino acid sequence of VL composed of CDR of VL of nonhuman animal antibody and FR of VL of arbitrary human antibody are constructed, and a humanized antibody expression vector is constructed and introduced into animal, fungus or bacterial cells, thereby expressing it.

Mouse-derived hybridoma antibodies can trigger immune side effects due to their constant regions being recognized by the human immune system, thereby inducing a human anti-mouse antibody (HAMA) response. Through humanization, the immunogenicity of murine antibodies can be eliminated or reduced. Humanized antibodies exhibit lower immunogenicity, thereby closely resembling natural human antibodies, and enhancing therapeutic efficacy and safety. In one example, compared with non-humanized antibodies with the same specificity (e.g., the murine antibody precursor before humanization), the humanized antibodies of this disclosure demonstrate reduced immunogenicity in human subjects. In the present invention, the hybridoma antibody SC27.105 (from WO2016073649A1) was humanized. In one example, the humanized antibodies (H1, P1, P2, P3, P4, M1, M2, etc.) of the present application exhibit low immunogenicity in human subjects, and, in particular, lower than that of the hybridoma antibody SC27.105. A low or reduced immunogenicity is characterized by a relief in measurable symptoms and the ability to provide long-term treatment with low and/or acceptable toxicity. Low or acceptable immunogenicity and/or high affinity, along with other suitable properties, can contribute to achieving therapeutic outcomes. Herein, “low or reduced immunogenicity” is defined as HAHA, HACA, or HAMA responses occurring in less than 90% of patients (e.g., below 80%, 70%, 60%, 50%, 40%, 30%, 20%, or even 10%).

The term “recombinant antibody” refers to antibodies produced by using recombinant DNA technology, such as antibodies or antibody fragments expressed in phage, yeast, or mammalian cell expression systems. This term also encompasses antibodies generated by synthesizing DNA molecules encoding the antibody and expressing the antibody protein from these DNA molecules; or synthesizing the amino acid sequence of the antibody, wherein the DNA or amino acid sequence is obtained using established recombinant DNA or amino acid synthesis techniques in the field. The antibodies of the present invention can be isolated by screening combinatorial libraries of antibodies with one or more desired activities. For example, various methods are known in the art for generating phage display libraries and screening such libraries for antibodies with desired binding properties. Such methods are reviewed in, for example, Hoogenboom et al., Methods in Molecular Biology 178:1-37 (O'Brien et al., Human Press, Totowa, NJ, 2001), and further described in, for example, McCafferty et al., Nature 348:552-554 (1991); Clackson et al., Nature 352:624-628 (1991); Marks et al., J. Mol. Biol. 222:581-597 (1992); Marks, Meth. Mol. Biol., 248:161-175 (Lo, ed., Human Press, Totowa, NJ, 2003); Sidhu et al., J. Mol. Biol. 338 (2): 299-310 (2004); Lee et al., J. Mol. Biol. 340 (5): 1073-1093 (2004); Fellouse, Proc. Natl. Acad. Sci. USA 101 (34): 12467-12472 (2004); and Lee et al., J. Immunol. Methods 284 (1-2): 119-132 (2004).

In certain phage display methods, the VH and VL gene libraries are separately cloned via polymerase chain reaction (PCR) and randomly recombined in a phage library, followed by screening the library for phages binding to the target antigen, as described by Winter et al. (Ann. Rev. Immunol. 12:433-455, 1994). Phages typically display antibody fragments as single-chain Fv (scFv) fragments or Fab fragments. Libraries derived from immunized sources provide high-affinity antibodies against the immunogen without constructing the hybridoma. Alternatively, naïve libraries (e.g., from humans) can be cloned to generate a single source of antibodies targeting diverse non-self and self-antigens without prior immunization, as reported by Griffiths et al. (EMBO J. 12:725-734, 1993). Finally, synthetic naïve libraries may also be prepared by cloning unrearranged V-gene segments from stem cells and using PCR primers containing random sequences to encode hypervariable CDR3 regions, followed by in vitro rearrangement, as described by Hoogenboom (J. Mol. Biol. 227:381-388, 1992). In some embodiments, amino acid sequence variants of antibodies are provided herein. The term “parent antibody” refers to an antibody disclosed in the present application or derived from such an antibody through processes like mutation or affinity maturation. The parent antibody may be a naturally occurring antibody, a variant thereof, or an engineered version. The parent antibody may refer to the antibody itself, a composition containing it, or its encoded amino acid sequence.

The term, “affinity-matured antibody” refers to an antibody with one or more alterations in one or more hypervariable regions (CDRs) compared with the parent antibody, thereby enhancing binding affinity to the antigen.

The term, “variant” refers to one or more biologically active polypeptides with substantially identical amino acid sequence (or encoded by a substantially identical nucleotide sequence) to the antibody provided in the present application. The variant exhibits the same or similar activity as the antibody described in the examples of the present application. For example, the variant may be a modified antibody or antibody variant based on the amino acid sequence of the antibody disclosed herein.

The term “variant antibody” or “antibody variant” refers to an antibody sequence that differ from the parent antibody sequence due to at least one amino acid modification. In this context, the variant antibody sequence preferably has at least about 80%, 81%, 82%, 83%, 84%, 85%, 86%, 87%, 88%, 89%, 90%, 91%, 92%, 93%, 94%, 95%, 96%, 97%, 98%, or 99% amino acid sequence identity to the parental antibody sequence. The antibody variant may refer to the antibody itself or a composition comprising said variant. The amino acid sequence variant of an antibody can be prepared by introducing suitable modifications into the nucleotide sequence encoding the antibody or through peptide synthesis. The term “amino acid modification” encompasses substitutions, additions, and/or deletions, “amino acid substitution” or “replacement” means replacing an amino acid at a specific position in the parent polypeptide sequence with another amino acid, “amino acid insertion” means adding an amino acid at a specific position in the parent polypeptide sequence, and “amino acid deletion” means removing an amino acid at a specific position in the parent polypeptide sequence. Any combination of deletions, insertions, and substitutions may be used to obtain the final construct, provided that the final construct retains desired characteristics, such as antigen-binding capability.

The term “modification” refers to an alteration in the state or structure of the protein or polypeptide of the present invention. Such modification can be chemical, structural, or functional in nature.

The term “conservative modification” or “conservative sequence modification” refer to an amino acid modification which does not significantly affect or alter the binding characteristics of the antibody or antibody fragment containing said amino acid sequence. Such conservative modifications comprise amino acid substitutions, insertions, and deletions. The modifications can be introduced into the antibody or antibody fragment of the invention using standard techniques known in the art, such as site-directed mutagenesis or PCR-mediated mutagenesis. A conservative amino acid substitution occurs when an amino acid residue is replaced by another residue with a similar side chain. Families of amino acid residues with similar side chains are defined in the art, as shown in Table 1.

TABLE 1
Families of Amino Acid Residues with Similar Side Chains

| | |
|---|---|
| Amino acids with basic side | Lys (K), Arg (R), His (H) |
| chains | |
| Amino acids with acidic side | Asp (D), Glu (E) |
| chains | |
| Amino acids with polar or | Asn (N), Ser(S), Thr (T), Tyr (Y), |
| neutral side chains | Cys (C), Trp (W), Met (M), Gln (Q) |
| Amino acids with nonpolar | Gly (G), Ala (A), Val (V), Leu (L), |
| side chains | Ile (I), Pro (P), Phe (F) |
| Amino acids with | Thr (T), Val (V), Ile (I) |
| β-branched side chains | |
| Amino acids with aromatic | Tyr (Y), Phe (F), Trp (W) |
| side chains | |

Therefore, one or more amino acid residues in the CDR region or framework region of the antibody of the present invention may be replaced with residues from the same side-chain family, and the retained function of the modified antibody (variant antibody) can be tested.

Non-conservative substitutions require replacing a member of one group with a member of another group.

A substitution variant involves replacing one or more hypervariable region or framework region (FR) residues of a parent antibody (e.g., a humanized or human antibody). Typically, the resulting variants selected for further study will exhibit altered (e.g., enhanced) biological properties relative to the parent antibody, such as an increased affinity or reduced immunogenicity, and/or will substantially retain certain biological properties of the parent antibody. An exemplary substitution variant is an affinity-matured antibody, which can be routinely generated using phage display-based affinity maturation techniques (such as those described herein). Briefly, one or more CDR or FR residues are mutated, and the variant antibodies are displayed on phage, and screened for specific biological activity (e.g., binding affinity)

Modifications (e.g., substitutions) can be introduced in the CDR regions to, for example, enhance antibody affinity. Such modifications may target CDR “hotspots”, which are residues encoded by codons that undergo high-frequency mutations during somatic hypermutation (see, e.g., Chowdhury, Methods Mol. Biol. 207:179-196 (2008)) and/or residues involved in antigen unbinding, followed by testing the binding affinity of the resulting variant VH or VL. Affinity maturation via constructing secondary libraries and reselection has been described in, for example, Hoogenboom et al., Methods in Molecular Biology 178:1-37 (O'Brien et al., eds., Humana Press, Totowa, NJ, 2001). In some embodiments of affinity maturation, the diversity is introduced into selected variable gene segments through methods such as error-prone PCR, chain shuffling, or oligonucleotide-directed mutagenesis. A secondary library is then generated and screened to identify antibody variants with desired affinity. An alternative approach for introducing diversity involves CDR-directed methods, wherein multiple CDR residues (e.g., 4-6 residues simultaneously) are randomized.

In certain embodiments, substitutions, insertions, or deletions may occur within one or more CDRs, provided that such alterations do not substantially reduce the antibody's ability to bind antigens. For example, conservative changes (e.g., conservative modifications as described herein) that do not significantly reduce binding affinity may be introduced into CDRs. Such alterations may occur, for instance, outside residues of the CDR that contact the antigen. In some embodiments of the variant VH and VL sequences provided above, each CDR is either unmodified or contains no more than one, two, or three amino acid substitutions.

In certain embodiments, the insertion of amino acid sequence comprises N-terminal and/or C-terminal fusions, ranging from a single residue to polypeptides containing one hundred or more residues, as well as the insertions of single or multiple amino acid residues inside the sequence. An example of terminal insertion comprises an antibody with an N-terminal methionyl residue. Other insertion variants of an antibody molecule involve fusing the N- or C-terminus to an enzyme or polypeptide, thereby extending the antibody's serum half-life.

Terms “anti-CLDN6 antibody”, “CLDN6-binding antibody”, “CLDN6 antibody”, and “CLDN6-recognizing antibody” refers to an antibody capable of binding CLDN6 with sufficient affinity, which can be used as diagnostic and/or therapeutic agents targeting CLDN6. In one embodiment, the anti-CLDN6 antibody binds to unrelated, non-CLDN6 proteins (e.g., CLDN4, CLDN9) at a level less than approximately 20%, 19%, 18%, 17%, 16%, 15%, 14%, 13%, 12%, 11%, or 10% of the binding of the antibody to CLDN6, as measured by enzyme-linked immunosorbent assay (ELISA). In one embodiment, the binding of the anti-CLDN6 antibody to CLDN4 or CLDN9 is comparable to the baseline binding level observed in negative control groups. In certain embodiments, the anti-CLDN6 antibody binds to an epitope of CLDN6 that is conserved across CLDN6s derived from different species.

The term “Chimeric T Cell Receptor” refers to recombinant polypeptides derived from various polypeptides comprising a TCR, capable of binding to surface antigens on target cells and interacting with other polypeptides of the intact TCR complex, typically localized on the T cell surface. A chimeric T cell receptor comprises a TCR subunit and an antigen-binding domain comprising human or humanized antibody domains, wherein the TCR subunit comprises at least a portion of the TCR extracellular domain, transmembrane domain, and the stimulatory domain of the intracellular signaling domain of the TCR intracellular region; the TCR subunit is effectively linked to the antibody domain, and the extracellular, transmembrane, and intracellular signaling domains of the TCR subunit are derived from CD3ε or CD3γ. Furthermore, the chimeric T cell receptor is integrated into the TCR expressed on T cells.

The term “T cell antigen coupler (TAC)” comprises 3 functional domains: (1) an antigen-binding domain, comprising single-chain antibodies, designed ankyrin repeat proteins (DARPins), or other targeting moieties; (2) an extracellular domain with a single-chain antibody binding to CD3, thereby bringing the TAC receptor into proximity with the TCR receptor; (3) a transmembrane domain and the intracellular region of the CD4 co-receptor, wherein the intracellular region links to the protein kinase LCK, thereby catalyzing the phosphorylation of immunoreceptor tyrosine-based activation motifs (ITAMs) in the TCR complex as the initial step of T cell activation.

The term “Chimeric Antigen Receptor” (CAR) comprises an extracellular antigen-binding domain, a transmembrane domain, and an intracellular signaling domain.

The term “transmembrane domain” or “transmembrane region” is linked to the extracellular antigen-binding domain. The transmembrane domain can be derived from natural or synthetic sources. When naturally sourced, it may be derived from any membrane-bound or transmembrane protein. On one hand, whenever a CAR binds to its target, the transmembrane domain transmits signals to the intracellular domain. The transmembrane domains comprise: α, β, γ, δ, or ζ chains of a T-cell receptor; CD28, CD3ε, CD45, CD4, CD5, CD8, CD9, CD16, CD22, CD33, CD37, CD64, CD80, CD86, CD134, CD137, CD154; IL-2 receptor p55 (α chain), p75 (β chain), or γ chain); and FcγRIII. For synthetic transmembrane domains, hydrophobic residues, such as leucine and valine may be incorporated. On one hand, a triplet of phenylalanine-tryptophan-valine can be present at both ends of the synthetic transmembrane domain.

In some cases, the transmembrane domain may comprise one or more additional amino acids adjacent to the transmembrane region, such as one or more amino acids related to the extracellular region of the protein from which the transmembrane domain is derived, and/or one or more amino acids related to the intracellular region of the protein from which the transmembrane domain is derived. In some cases, the transmembrane domain can be linked to the extracellular region of the CAR via a “hinge region” or “hinge domain”. The use of a hinge region provides greater flexibility and accessibility to the extracellular antigen-binding domain. The hinge region may comprise up to 300 amino acids, preferably 10-100 amino acids, and most preferably 25-50 amino acids. The hinge region can be derived from naturally occurring molecules, such as the entire or partial extracellular regions of CD8, CD4, CD28, FcγRIII, or IgG1/IgG4, or from the entire or partial constant region of an antibody. Alternatively, the hinge region may also be a synthetic sequence.

The term “intracellular signaling region” or “intracellular signaling domain” or “intracellular signaling transduction region” or “intracellular signaling transduction domain” refers to the intracellular portion of a CAR molecule, responsible for activating at least one normal effector function of immune cells into which a CAR has been introduced. The intracellular signaling domain generates signals that promote cytotoxic activity and helper functions (comprising cytokine secretion) in CAR-expressing cells (e.g., CAR-T cells). A “signaling domain” or “signaling transduction domain” refers to a protein segment that transduces effector signals and directs the cell to perform specific functions. The signaling domain of a CAR can be a cytoplasmic sequence of a T-cell receptor (TCR), a cytoplasmic sequence of a co-receptors that cooperate in signal transduction upon antigen receptor engagement, and any derivatives or variants of these sequences, as well as any synthetic sequences with equivalent functionality.

The intracellular signaling domain may comprise functional signaling domains of stimulatory molecules (also referred to as stimulatory or primary signaling molecules) and/or costimulatory molecules. In one embodiment, the intracellular signaling domain may comprise a primary signaling molecule, for example, a primary signaling molecule derived from the domains of a molecule responsible for the first stimulus or antigen-dependent stimulation. In an embodiment, signals generated solely through the TCR are insufficient to fully activate T cells, and a secondary and/or costimulatory signal is necessary. The intracellular signaling domain may comprise a costimulatory intracellular signaling domain. For example, a costimulatory intracellular signaling domain comprise those domains derived from molecules responsible for costimulatory signals or antigen-independent stimulation. The intracellular signaling domain may comprise the entire intracellular portion of the molecule from which it is derived, the complete native intracellular signaling domain of that molecule, or a functional fragment thereof. For example, the stimulatory molecule can be the ζ-chain which binds to the T-cell receptor complex. For example, the cytoplasmic signaling domain may further comprise functional signaling domains from one or more costimulatory molecules, such as the intracellular sequences of 4-1BB (i.e., CD137), CD27, and/or CD28.

In the present invention, in one aspect, the CAR comprises a chimeric fusion protein, which comprises an extracellular antigen recognition domain, a transmembrane domain, and an intracellular signaling domain. The intracellular signaling domain comprises a functional signaling domain derived from a stimulatory molecule. In one aspect, the CAR comprises a chimeric fusion protein, which comprises an extracellular antigen recognition domain, a transmembrane domain, and an intracellular signaling domain. The intracellular signaling domain comprises both a functional signaling domain derived from a co-stimulatory molecule and a functional signaling domain derived from a stimulatory molecule. In one aspect, the CAR comprises a chimeric fusion protein, which comprises an extracellular antigen recognition domain, a transmembrane domain, and an intracellular signaling domain. The intracellular signaling domain contains at least two functional signaling domains derived from one or more costimulatory molecules and a functional signaling domain derived from a stimulatory molecule. In one aspect, the CAR optionally comprises a “leader sequence” at the amino (N) terminus of the CAR fusion protein. In one aspect, the CAR further comprises a leader sequence at the N-terminus of the extracellular antigen recognition domain, wherein the leader sequence is optionally cleaved from the antigen recognition domain (e.g., scFv) during the cellular processing and localization to the cell membrane.

The term “leader sequence” (also known as a signal peptide) is operably linked to the CAR nucleic acid sequence and located to guide the newly synthesized polypeptide into the secretory pathway of the cell. Typically, the leader sequence is located at the 5′ end of the nucleic acid sequence encoding the polypeptide. Signal peptides comprise naturally occurring signal sequences of CAR proteins or synthetic non-naturally occurring signal sequences. In some embodiments, the signal peptide is selected from CD8α, GM-CSF receptor α, and IgG1 heavy chain.

The term “primary signaling molecule” or “stimulatory molecule” regulates the initial activation of the TCR complex in a stimulatory manner. Generally, the primary signaling is triggered by, for example, the binding of the TCR/CD3 complex to a peptide-loaded MHC molecule, thereby mediating T cell responses (comprising but not limited to proliferation, activation, differentiation, etc.). Primary signaling molecules that act in a stimulatory manner may contain signaling motifs, such as immunoreceptor tyrosine-based activation motifs (ITAMs). Examples of functional signaling domains (primary signaling domains) of primary signaling molecules containing ITAMs particularly useful in the present invention comprise, but not limited to sequences derived from CD3ζ, FcRγ, FcRβ, CD3γ, CD3δ, CD3ε, CD5, CD22, CD79a, CD79b, CD278 (also known as “ICOS”), CD66d, DAP10, and DAP12. In the CAR of the present invention, the intracellular signaling domain in any one or more CARs of the present invention comprises an intracellular signaling sequence, such as the primary signaling domain of CD3ζ.

The term “costimulatory signaling domain” or “costimulatory signaling region” or “costimulatory molecule” generally refers to the intracellular domain of a costimulatory molecule capable of binding to cell stimulatory signaling molecules, such as TCR/CD3, thereby inducing signals for T cell proliferation and/or upregulation or downregulation of key molecules. Costimulatory molecules are typically cognate binding partners on T cells that specifically bind costimulatory ligands, thereby mediating costimulatory responses in T cells, comprising but not limited to the proliferation. Costimulatory molecules are cell surface molecules or ligands thereof that are required for an effective immune response but are not antigen receptors. The intracellular costimulatory signaling domain can be derived from the intracellular portion of a costimulatory molecule. Costimulatory molecules may be present in following protein families: TNF receptor proteins, immunoglobulin-like proteins, cytokine receptors, integrins, signaling lymphocyte activation molecules (SLAM proteins), and activating NK cell receptors. Costimulatory molecules comprise but are not limited to MHC class I molecules, BTLA, Toll ligand receptors, OX40, CD2, CD27, CD28, CD5, ICAM-1, LFA-1 (CD11a/CD18), 4-1BB (CD137), GITR, CD30, CD40, ICOS, BAFFR, HVEM, CD7, LIGHT, NKG2C, SLAMF7, NKp80, CD160, B7-H3, and ligands specifically binding CD83.

The term “CD3ζ (also known as CD3Zeta)” or “TCRζ” or “ζ chain” is defined as the protein provided by GenBank accession number BAG36664.1 or equivalent residues from non-human species such as mice, rodents, monkeys, apes, etc. The “CD3ζ domain” is defined as the amino acid residues from the cytoplasmic domain of the ζ chain sufficient to functionally transmit the initial signal required for T cell activation. The cytoplasmic domain of CD3ζ comprises residues 52 to 164 of GenBank accession number BAG36664.1 or functional orthologs thereof from non-human species such as mice, rodents, monkeys, apes, etc. For example, the amino acid sequence of CD3ζ is as shown in SEQ ID NO: 24.

The term “4-1BB (also known as CD137)” refers to a member of the TNFR superfamily, which has the amino acid sequence of GenBank Acc. No. AAA62478.2 or equivalent residues from non-human species such as mice, rodents, monkeys, apes, etc. The “4-1BB costimulatory domain” is defined as residues 214-255 of GenBank Acc. No. AAA62478.2 or equivalent residues from non-human species such as mice, rodents, monkeys, apes, etc. For example, the amino acid sequence of the 4-1BB costimulatory domain is as shown in SEQ ID NO: 26.

The term “chemokine” refers to a polypeptide with a molecular weight of 8~10 kDa, which is the largest cytokine family, primarily functions to recruit monocytes, neutrophils, lymphocytes, etc., from the blood into specific lymphoid organs and tissues as well as sites of infection.

The term “chemokine receptor” refers to a class of seven-transmembrane G protein-coupled receptors (GPCRs) that mediate chemokine function, and are typically expressed on immune cells, neutrophils, endothelial cells, etc. Chemokines and chemokine receptors play important roles in mediating cell migration, proliferation, and defense against pathogen invasion, and are closely related to inflammation and cancer development in immune environments.

The term “safety switch” is designed to enhance the safety of CAR-T therapy by providing a rapid and reversible “off” or “on” switch to minimize treatment-related toxicity. Despite the outstanding clinical profile of CAR-T cell therapy, severe cytokine release syndrome (CRS) and other potentially fatal side effects can occur when tumor burden is unpredictable and T cell activity is uncontrolled. To control toxicity, severe CRS shall be appropriately monitored by using a CRS grading system and small molecule-based safety switches shall be used for precise regulation.

### DETAILED DESCRIPTION OF THE INVENTION

### Antibody

In the context, it is described that humanized anti-CLDN6-specific antibodies were obtained using conventional humanized antibody preparation techniques in the art. Mutants of humanized anti-CLDN6-specific antibodies were generated through CDR randomization and phage screening technologies. These molecules exhibit fine specificity. For example, the antibody recognizes only CLDN6 and 293T cells, OVCAR3 cells, and OV90 cells expressing CLDN6, but does not recognize cells expressing CLDN4, CLDN9, or a combination thereof. Unless otherwise specified, CLDN6 in this invention refers to human CLDN6.

In some embodiments, the present invention comprises antibodies with scFv sequences fused to one or more heavy chain constant regions to form bivalent proteins with human immunoglobulin Fc regions, thereby increasing the overall affinity and stability of the antibody. Additionally, the Fc portion allows direct conjugation of other molecules (comprising but not limited to fluorescent dyes, cytotoxins, radioisotopes, etc.) to, for example an antibody used in antigen quantification studies, so that the antibody is immobilized for the affinity measurement, targeted delivery of therapeutics, testing Fc-mediated cytotoxicity with immune effector cells, and the like.

The results provided herein highlight the specificity, sensitivity, and use of the antibody of the present invention in targeting CLDN6.

The antibody or antibody fragments of the present invention are based on single-chain antibody fragments (scFv) obtained using humanized antibody preparation techniques and phage screening. The amino acid sequences of the scFvs confer the antibody or antibody fragments specificity to CLDN6 and form the basis of all antibodies disclosed herein. Therefore, the scFvs can be used to design a range of different “antibodies or antibody fragments”, comprising, for example, full-length antibodies, fragments thereof such as F(ab′) 2, fusion proteins, multivalent antibodies (i.e., antibodies with more than one specificity for the same or different antigens), such as bispecific T-cell engagers (BiTEs), triabodies, etc. (see Cuesta et al., Multivalent antibodies: when design surpasses evolution, Trends in Biotechnology 28:355-362, 2010).

In a specific embodiment, the invention provides a full-length antibody, the heavy and light chains of which may be full-length (e.g., the antibody may comprise at least one, preferably two, complete heavy chains, and at least one, preferably two, complete light chains) or may comprise antigen-binding portions (Fab, F(ab′) 2, Fv, or scFv). In other embodiments, the heavy chain constant region of the antibody is selected from, for example, IgG1, IgG2, IgG3, IgG4, IgM, IgA1, IgA2, IgD, and IgE. The selection of antibody type will depend on the desired immune effector function of the designed antibody. In constructing recombinant immunoglobulins, suitable amino acid sequences for constant regions of various immunoglobulin isotypes and methods for producing a wide variety of antibodies are well known to a skilled person.

The invention provides antibodies or antigen-binding fragments recognizing CLDN6, comprising a heavy chain variable region containing HCDR1 as shown in GYYMN (SEQ ID NO: 35); and/or HCDR2 as shown in EINPATGSTTYNQKFKA (SEQ ID NO: 36); and/or HCDR3 as shown in RDYYX₁GSX₂X₃YAX4DY (SEQ ID NO: 52), wherein X₁ is Y or L, X₂ is G or N, X₃ is F or S, X₄ is M or L; and/or comprising a light chain variable region containing LCDR1 as shown in QASQSVSNNLN (SEQ ID NO: 38); and/or LCDR2 as shown in GASKLED (SEQ ID NO: 39); and/or LCDR3 as shown in X₅QHRX₆X₇WT (SEQ ID NO: 53), wherein X₅ is L or Q, X₆ is Y or F, and X₇ is L or M.

In certain embodiments, the antibody or antigen-binding fragment may comprise at least one CDR from the heavy chain variable region; and/or the antibody or antigen-binding fragment may comprise at least one CDR from the light chain variable region. In certain embodiments, the antibody or antigen-binding fragment may comprise one, two, or three CDRs from the heavy chain variable region. In certain embodiments, the antibody or antigen-binding fragment may comprise one, two, or three CDRs from the light chain variable region.

In certain embodiments, the antibody may comprise one, two, or three CDRs from the heavy chain variable region and one, two, or three CDRs from the light chain variable region. For example, it may comprise one CDR from the heavy chain variable region and one, two, or three CDRs from the light chain variable region; or it may comprise two CDRs from the heavy chain variable region and one, two, or three CDRs from the light chain variable region; or it may comprise three CDRs from the heavy chain variable region and one, two, or three CDRs from the light chain variable region; or it may comprise three CDRs from the heavy chain variable region and three CDRs from the light chain variable region.

Therefore, the antibody or antigen-binding fragment of the invention comprises a heavy chain variable region, HCDR3 of which has at least 95%, 96%, 97%, 98%, or 99% amino acid sequence identity with the HCDR3 shown in SEQ ID NO: 41; and/or
the antibody or antigen-binding fragment of the invention comprises a light chain variable region, LCDR3 of which has at least 60%, 65%, 70%, 75%, 80%, 85%, 90%, 91%, 92%, 93%, 94%, 95%, 96%, 97%, 98%, or 99% amino acid sequence identity with the LCDR3 shown in SEQ ID NO: 42, 43, or 44.

In certain embodiments, the antibody may comprise the heavy chain variable region and/or the light chain variable region. For example, the heavy chain variable region may comprise a heavy chain CDR1 (HCDR1), which may comprise the amino acid sequence shown in SEQ ID NO: 35. For example, the heavy chain variable region may comprise a heavy chain CDR2 (HCDR2), which may comprise the amino acid sequence shown in SEQ ID NO: 36. For example, the heavy chain variable region may comprise a heavy chain CDR3 (HCDR3), which may comprise the amino acid sequence shown in SEQ ID NO: 37 or 41. For example, the heavy chain variable region may comprise HCDR1 and HCDR3, wherein HCDR1 may comprise the amino acid sequence shown in SEQ ID NO: 35, and HCDR3 may comprise the amino acid sequence shown in SEQ ID NO: 37 or 41. For example, the heavy chain variable region may comprise HCDR1 and HCDR2, wherein HCDR1 may comprise the amino acid sequence shown in SEQ ID NO: 35, and HCDR2 may comprise the amino acid sequence shown in SEQ ID NO: 36. For example, the heavy chain variable region may comprise HCDR2 and HCDR3, wherein HCDR2 may comprise the amino acid sequence shown in SEQ ID NO: 36, and HCDR3 may comprise the amino acid sequence shown in SEQ ID NO: 37 or 41. For example, the heavy chain variable region may comprise HCDR1, HCDR2, and HCDR3, wherein HCDR1 may comprise the amino acid sequence shown in SEQ ID NO: 35, HCDR2 may comprise the amino acid sequence shown in SEQ ID NO: 36, and HCDR3 may comprise the amino acid sequence shown in SEQ ID NO: 37 or 41. For example, the light chain variable region may comprise a light chain CDR1 (LCDR1), which may comprise the amino acid sequence shown in SEQ ID NO: 38. For example, the light chain variable region may comprise a light chain CDR2 (LCDR2), which may comprise the amino acid sequence shown in SEQ ID NO: 39. For example, the light chain variable region may comprise a light chain CDR3 (LCDR3), which may comprise the amino acid sequence shown in SEQ ID NO: 40, 42, 43, or 44. For example, the light chain variable region may comprise LCDR1 and LCDR3, wherein LCDR1 may comprise the amino acid sequence shown in SEQ ID NO: 38, and LCDR3 may comprise the amino acid sequence shown in SEQ ID NO: 40, 42, 43, or 44. For example, the light chain variable region may comprise LCDR1 and LCDR2, wherein LCDR1 may comprise the amino acid sequence shown in SEQ ID NO: 38, and LCDR2 may comprise the amino acid sequence shown in SEQ ID NO: 39. For example, the light chain variable region may comprise LCDR2 and LCDR3, wherein LCDR2 may comprise the amino acid sequence shown in SEQ ID NO: 39, and LCDR3 may comprise the amino acid sequence shown in SEQ ID NO: 40, 42, 43, or 44. For example, the light chain variable region may comprise LCDR1, LCDR2, and LCDR3, wherein LCDR1 may comprise the amino acid sequence shown in SEQ ID NO: 38, LCDR2 may comprise the amino acid sequence shown in SEQ ID NO: 39, and LCDR3 may comprise the amino acid sequence shown in SEQ ID NO: 40, 42, 43, or 44.

In certain embodiments, the antibody may comprise the heavy chain variable region and the light chain variable region. For example, the antibody comprises the HCDR1 shown in SEQ ID NO: 35, the HCDR2 shown in SEQ ID NO: 36, the HCDR3 shown in SEQ ID NO: 37, the LCDR1 shown in SEQ ID NO: 38, the LCDR2 shown in SEQ ID NO: 39, and the LCDR3 shown in SEQ ID NO: 40; or the antibody comprises the HCDR1 shown in SEQ ID NO: 35, the HCDR2 shown in SEQ ID NO: 36, the HCDR3 shown in SEQ ID NO: 41, the LCDR1 shown in SEQ ID NO: 38, the LCDR2 shown in SEQ ID NO: 39, and the LCDR3 shown in SEQ ID NO: 40; or the antibody comprises the HCDR1 shown in SEQ ID NO: 35, the HCDR2 shown in SEQ ID NO: 36, the HCDR3 shown in SEQ ID NO: 37, the LCDR1 shown in SEQ ID NO: 38, the LCDR2 shown in SEQ ID NO: 39, and the LCDR3 shown in SEQ ID NO: 42; or the antibody comprises the HCDR1 shown in SEQ ID NO: 35, the HCDR2 shown in SEQ ID NO: 36, the HCDR3 shown in SEQ ID NO: 37, the LCDR1 shown in SEQ ID NO: 38, the LCDR2 shown in SEQ ID NO: 39, and the LCDR3 shown in SEQ ID NO: 43; or the antibody comprises the HCDR1 shown in SEQ ID NO: 35, the HCDR2 shown in SEQ ID NO: 36, the HCDR3 shown in SEQ ID NO: 41, the LCDR1 shown in SEQ ID NO: 38, the LCDR2 shown in SEQ ID NO: 39, and the LCDR3 shown in SEQ ID NO: 42; or the antibody comprises the HCDR1 shown in SEQ ID NO: 35, the HCDR2 shown in SEQ ID NO: 36, the HCDR3 shown in SEQ ID NO: 41, the LCDR1 shown in SEQ ID NO: 38, the LCDR2 shown in SEQ ID NO: 39, and the LCDR3 shown in SEQ ID NO: 43; or the antibody comprises the HCDR1 shown in SEQ ID NO: 35, the HCDR2 shown in SEQ ID NO: 36, the HCDR3 shown in SEQ ID NO: 37, the LCDR1 shown in SEQ ID NO: 38, the LCDR2 shown in SEQ ID NO: 39, and the LCDR3 shown in SEQ ID NO: 44. In a specific embodiment, the HCDR2 of the antibody or antigen-binding fragment of the present invention has the amino acid sequence shown in SEQ ID NO: 36.

The CDRs can be determined using numbering systems selected from, for example, Kabat, Chothia, IMGT, Gelfand, Aho, and AbM. The antibodies provided in the invention may comprise CDR sequences or combinations thereof determined by any of the above numbering systems. It is not necessary to determine the CDRs in the heavy and/or light chains of the antibody by the same numbering system. For example, one or some CDRs in an antibody may be determined by the Kabat system, while other CDRs may be determined by any of the above numbering systems or combinations thereof. In certain embodiments, the CDRs of the antibody may be determined by a single numbering system, for example, the Kabat numbering system; or the Chothia numbering system; or the IMGT numbering system; or the Gelfand numbering system; or the Aho numbering system; or the AbM numbering system.

In certain embodiments, the heavy chain variable region comprises the amino acid sequence shown in SEQ ID NO: 1 or 5 or a variant thereof.

In certain embodiments, the light chain variable region comprises the amino acid sequence shown in SEQ ID NO: 3, 7, 9, or 11 or a variant thereof.

In certain embodiments, the heavy chain variable region comprises the amino acid sequence shown in SEQ ID NO: 1 or 5 or a variant thereof, and the light chain variable region comprises the amino acid sequence shown in SEQ ID NO: 3, 7, 9, or 11 or a variant thereof. For example, the heavy chain variable region comprises the amino acid sequence shown in SEQ ID NO: 1 or a variant thereof, and the light chain variable region comprises the amino acid sequence shown in SEQ ID NO: 3, 7, 9, or 11 or a variant thereof, for example, the heavy chain variable region comprises the amino acid sequence shown in SEQ ID NO: 5 or a variant thereof, and the light chain variable region comprises the amino acid sequence shown in SEQ ID NO: 3, 7, 9, or 11 or a variant thereof.

In certain embodiments, the heavy chain variable region comprises the amino acid sequence shown in SEQ ID NO: 1, and the light chain variable region comprises the amino acid sequence shown in SEQ ID NO: 3. For example, the heavy chain variable region comprises the amino acid sequence shown in SEQ ID NO: 1, and the light chain variable region comprises the amino acid sequence shown in SEQ ID NO: 7. For example, the heavy chain variable region comprises the amino acid sequence shown in SEQ ID NO: 1, and the light chain variable region comprises the amino acid sequence shown in SEQ ID NO: 9. For example, the heavy chain variable region comprises the amino acid sequence shown in SEQ ID NO: 5, and the light chain variable region comprises the amino acid sequence shown in SEQ ID NO: 3. For example, the heavy chain variable region comprises the amino acid sequence shown in SEQ ID NO: 5, and the light chain variable region comprises the amino acid sequence shown in SEQ ID NO: 7. For example, the heavy chain variable region comprises the amino acid sequence shown in SEQ ID NO: 5, and the light chain variable region comprises the amino acid sequence shown in SEQ ID NO: 9. For example, the heavy chain variable region comprises the amino acid sequence shown in SEQ ID NO: 1, and the light chain variable region comprises the amino acid sequence shown in SEQ ID NO: 11.

In certain embodiments, the invention provides antibodies recognizing CLDN6, comprising a heavy chain variable region, wherein the heavy chain variable region comprises the amino acid sequence shown in SEQ ID NO: 1 or 5 or a variant thereof.

In certain embodiments, the invention provides antibodies recognizing CLDN6, comprising a light chain variable region, wherein the light chain variable region comprises the amino acid sequence shown in SEQ ID NO: 3, 7, 9, or 11 or a variant thereof.

In certain embodiments, the invention provides antibodies recognizing CLDN6, comprising the above heavy chain variable region and light chain variable region.

Given that each of these heavy chain variable region and light chain variable region sequences can bind CLDN6, the heavy and light chain variable region sequences can be “mixed and matched” to produce anti-CLDN6 binding molecules of the invention.

In certain embodiments, the invention provides variants of antibodies that bind CLDN6 or variants of fragments thereof. Such variants comprise a heavy chain variable region, wherein the HCDR1, HCDR2, and HCDR3 have at least 85%, 90%, 91%, 92%, 93%, 94%, 95%, 96%, 97%, 98%, or 99% amino acid sequence identity with the HCDR1, HCDR2, and HCDR3 shown in SEQ ID NO: 35, 36 and 37. Such variants may further comprise a light chain variable region, wherein the LCDR1, LCDR2, and LCDR3 have at least 85%, 90%, 91%, 92%, 93%, 94%, 95%, 96%, 97%, 98%, or 99% amino acid sequence identity with the LCDR1, LCDR2, and LCDR3 shown in SEQ ID NO: 38, 39 and 40.

Further, variants of the antibodies of the invention or variants of fragments thereof comprise heavy and/or light chain variable regions that have at least 80%, preferably at least 85%, more preferably at least 90%, most preferably at least 95%, particularly 96%, more particularly 97%, even more particularly 98%, and most particularly 99% (comprising, for example, 80%, 81%, 82%, 83%, 84%, 85%, 86%, 87%, 88%, 89%, 90%, 91%, 92%, 93%, 94%, 95%, 96%, 97%, 98%, 99%, and 100%) sequence identity with the heavy or light chain variable regions of the antibodies of the invention. Variants can be obtained from the parent antibodies described in the present application through methods such as yeast library screening, phage library screening, point mutation, etc.

In certain embodiments, the invention provides antibodies that specifically bind CLDN6, wherein the antibodies are full antibodies, scFvs, single-domain antibodies, Fab fragments, Fab′ fragments, Fv fragments, F(ab′) 2 fragments, Fd fragments, dAbs, or multifunctional antibodies.

In certain embodiments, the antibodies are hybridoma antibodies, chimeric antibodies, humanized antibodies, or fully human antibodies.

In certain embodiments, the antibodies are monoclonal antibodies.

### Antibody Assays

The anti-CLDN6 antibodies provided herein can be identified, screened, or characterized for physical/chemical properties and/or biological activities thereof using a variety of assays known in the art, comprising, for example, ELISA, Biacore, Western blotting, and flow cytometry. Suitable assays are described in detail in the Examples.

The term “affinity” refers to the total strength of non-covalent interactions between a single binding site of a molecule (e.g., an antibody) and its binding partner (e.g., an antigen). Unless otherwise indicated, “binding affinity” as used herein refers to the intrinsic binding affinity, reflecting a 1:1 interaction between the members of a binding pair (e.g., an antibody and an antigen). The affinity of molecule X for its ligand Y can generally be represented by the dissociation constant (Kd). The affinity can be measured by conventional methods known in the art, comprising Biacore assays for antibody affinity. For example, the “affinity” of the antibody herein for CLDN6 is represented as the KD of the antibody. The KD of an antibody refers to the equilibrium dissociation constant of the antibody-antigen interaction. The higher the KD value of an antibody binding to its antigen, the weaker its binding affinity for that specific antigen. For example, the “affinity” of the antibody herein for the CLDN antigen is represented as the EC50 of the antibody.

The term “EC50”, the concentration for 50% of maximal effect, refers to the concentration that causes 50% of the maximal effect.

Various methods for determining antibody binding affinity are known in the art. In one embodiment, surface plasmon resonance is adopted in the method for determining the binding affinity. Surface plasmon resonance is an optical phenomenon that can be used to analyze real-time biospecific interactions by, for example, detecting changes in protein concentration in the substrate of a biochip using a Biacore system.

“Non-significant binding” is characterized by a reduced affinity of the antibody for the antigen, faster dissociation rates, and/or lower binding signals, particularly faster dissociation rates and/or lower binding signals. In one example, “non-significant binding” refers to the level of binding of an antibody to an antigen protein or polypeptide that is not statistically significantly higher than the background; and the background is the binding level detected in the absence of the antibody or in the presence of a negative control protein (e.g., an isotype control antibody). For example, a biosensor analysis (e.g., Biacore) is adopted to detect the binding level of an antibody to an antigen protein or polypeptide or to detect the binding level of an antibody to an antigen protein or polypeptide expressed on the cell surface. In one example, PBS is used instead of the antibody to detect antigen binding levels. In one example, the antibody of the invention binds CLDN6. In one example, the antibody of the invention does not significantly bind CLDN4 or CLDN9.

### Antigen

The term “antigen” or “Ag” refers to a substance recognized and specifically bound by an antigen-binding unit. Antigens may comprise peptides, proteins, glycoproteins, polysaccharides, lipids, fragments, and combinations thereof. Non-limiting exemplary antigens comprise tumor antigens or pathogen antigens. “Antigen” may also refer to a molecule eliciting an immune response. This immune response may involve antibody production, activation of specific immunologically-competent cells, or both. A skilled person will appreciate that any macromolecule, comprising virtually all proteins or peptides, can serve as an antigen. Additionally, antigens may be derived from recombinant DNAs or genomic DNAs. Any DNA containing a nucleotide sequence or partial nucleotide sequence encoding a protein capable of inducing an immune response can encode an antigen. A skilled person will appreciate that it is not necessary to encode an antigen solely by the full-length nucleotide sequence of a gene. This comprises, but is not limited to, the use of partial nucleotide sequences from more than one gene, which can be arranged in various combinations to encode polypeptides capable of eliciting the desired immune response. Furthermore, antigens may also be non-gene-encoded. Antigens can be synthetically produced, derived from biological samples, or be macromolecules other than polypeptides. The biological samples may comprise, but are not limited to, tissue samples, tumor samples, cells or fluids, and other biological components.

The term “epitope” refers to an antigen or part of an antigen that can be recognized by antibodies, B cells, T cells, or engineered cells. For example, an epitope may be a tumor epitope or pathogen epitope recognized by an antibody; and an antibody can recognize multiple epitopes within an antigen. There also may be mutations in an epitope.

The term “antigenic determinant”, also known as “antigenic epitope”, “epitope”, or “antigenic determinant”, comprises any determinant or region capable of being bound by an antibody. An antigenic epitope is the region of an antigen bound by an antibody targeting that antigen, comprising specific amino acids that directly contact the antibody. For example, an antigenic epitope may comprise a continuous sequence from the CLDN6 protein sequence or a discontinuous three-dimensional structure of the CLDN6 protein sequence. For example, the antigen used herein is human CLDN6.

### Immunoconjugates

The present invention also provides an immunoconjugate comprising the antibody described herein and one or more functional molecules linked thereto. The antibody provided in the present invention has been described earlier, and the conjugate provided in the invention encompasses all technical solutions thereof. The antibody and the functional molecule may form an immunoconjugate through covalent linkage, conjugation, attachment, cross-linking, or other means.

The terms “link”, “connect” or “fuse” are used interchangeably herein. They generally refer to the connection of two or more chemical elements or components by any means, comprising chemical conjugation or recombinant methods. “In-frame fusion” refers to the connection of two or more open reading frames (ORFs) in a manner that maintains the correct reading frame of the original ORF, thereby forming a continuous longer ORF. Therefore, the resulting recombinant fusion protein is a single protein comprising two or more fragments corresponding to the polypeptides encoded by the original ORFs (these fragments are not normally linked in their natural state). Although the reading frame is continuous throughout the fusion fragment, these fragments may be physically or spatially separated by, for example, an in-frame linker sequence (e.g., a “flexon”).

The functional molecule is selected from: molecules targeting tumor surface markers, molecules inhibiting tumors, molecules targeting surface markers of immune cells, or detectable markers. In some embodiments, the molecule targeting tumor surface markers may be an antibody or ligand that binds to tumor surface markers and can synergize with the antibody of the present invention, thereby more precisely targeting tumor cells.

In some embodiments, the molecule inhibiting tumors comprises cytotoxic agents, which comprise but are not limited to: radioactive isotopes, chemotherapeutic agents, growth inhibitors, enzymes and their fragments, antibiotics, and toxins. In some embodiments, the molecule inhibiting tumors is an anti-tumor cytokine, which comprises but are not limited to: IL-2, IL-7, IL-12, IL-15, type I IFN, TNF-α.

In some embodiments, the molecule targeting surface markers of immune cells is an antibody or ligand that binds to surface markers of immune cells, capable of recognizing immune cells and delivering the antibody of the present invention to immune cells, while the antibody can target immune cells to tumor cells, thereby inducing immune cells to specifically kill tumor cells. The immune cell surface markers are selected from CD3, CD6, CD28, NKG2A, NKG2C, NKG2D, CD94, CD159a, CD159c, CD158, CD56, LIR/ILT2, CD244, CD226, CD2, CD16, CD161, and the immune cells are selected from T cells, NK cells, and NKT cells. In one embodiment, the molecule targeting immune cell surface markers is an antibody binding to T cell surface markers, which forms a bi-functional antibody involving T cells with the antibody of the present invention.

In some embodiments, the detectable marker comprises but is not limited to: fluorescent markers, chromogenic markers; for example, enzymes, prosthetic groups, fluorescent materials, luminescent materials, bioluminescent materials, radioactive materials, positron-emitting metals, and non-radioactive paramagnetic metal ions. The markers used for the detection, analysis, or diagnostic purposes depend on the specific detection/analysis/diagnostic technique and/or method employed, such as immunohistochemical staining of (tissue) samples, flow cytometry, etc.

The immunoconjugate comprises antibody-drug conjugates (ADCs), wherein the antibody is conjugated to one or more agents, comprising but not limited to maytansinoids, auristatins such as monomethyl auristatin drug moieties DE and DF (MMAE and MMAF), dolastatins, calicheamicins or derivatives thereof, anthracycline antibiotics (e.g., daunorubicin or doxorubicin), methotrexate, vindesine, taxanes (e.g., docetaxel, paclitaxel, larotaxel, tesetaxel, and oxtataxel), trichothecenes, and CC1065. Another aspect of the present invention provides nucleic acid molecules encoding the immunoconjugates of the present invention. Once the relevant sequences are obtained, they can be produced in large quantities using recombinant methods, typically by cloning into vectors, introducing into cells, and isolating the relevant sequences from proliferated host cells using conventional methods.

The present invention also relates to vectors comprises the appropriate DNA sequences as said above and suitable promoters or control sequences. These vectors can be used to transform appropriate host cells to enable protein expression. Host cells may be prokaryotic, such as bacterial cells; or lower eukaryotic, such as yeast cells; or higher eukaryotic, such as mammalian cells.

### Chimeric Receptor

The present invention also provides a chimeric receptor, which generally refers to a product expressed by a fusion molecule produced by genetically recombining DNA fragments or cDNAs corresponding to proteins from different sources, may comprise an extracellular domain, transmembrane domain, and intracellular domain, and the extracellular domain comprises an antigen-binding domain. In one embodiment, the extracellular domain comprises the antibody provided in the present invention, which has been described in detail earlier, and the chimeric receptor provided in the invention encompasses all technical solutions thereof. The chimeric receptors comprise but are not limited to: chimeric antigen receptors (CARs), chimeric T cell receptors, T cell antigen couplers (TACs), and synthetic polypeptide receptors (synNotch).

In one embodiment, cells expressing the CAR can be enabled to target the target antigen by engineering an antigen-binding domain that specifically binds to a target antigen into a CAR.

In some embodiments, the chimeric receptor of the present invention is a chimeric antigen receptor (CAR). The chimeric antigen receptor typically comprises an extracellular antigen-binding region or antibody. In some embodiments, the extracellular antigen-binding region may be fully human. In other cases, the extracellular antigen-binding region may be humanized. In other cases, the extracellular antigen-binding region may be murine, or the chimera in the extracellular antigen-binding region may be comprise amino acid sequences from at least two different animals. In some embodiments, the extracellular antigen-binding region may be non-human.

In certain embodiments, the chimeric antigen receptor may also be designed to comprise multiple antigen-binding regions, comprising single-chain variable fragments (scFv) derived from antibodies, fragment antigen-binding regions (Fab) selected from libraries, single-domain fragments, or natural ligands binding to their homologous receptors. In some embodiments, the extracellular antigen-binding region may comprise scFv, Fab, or natural ligands, and any derivatives thereof. The extracellular antigen-binding region may refer to a molecule other than an intact antibody, which may comprise a portion of the intact antibody and can bind the antigen recognized by the intact antibody. Examples of antibody fragments comprise but are not limited to Fv, Fab, Fab′, Fab′-SH, F(ab′) 2; diabodies, linear antibodies; single-chain antibody molecules (e.g., scFv); and multispecific antibodies formed from antibody fragments. The extracellular antigen-binding region, such as scFv, Fab or natural ligand, may be part of a CAR that determines antigen specificity. The extracellular antigen-binding region may bind any complementary target. The extracellular antigen-binding region may be derived from an antibody with a known variable region sequence. The extracellular antigen-binding region may be obtained from antibody sequences derived from available murine hybridomas. Alternatively, the extracellular antigen-binding region may be obtained from whole-exome sequencing of tumor cells or primary cells, such as tumor-infiltrating lymphocytes (TILs).

In some embodiments, the binding specificity of the extracellular antigen-binding region of a CAR may be determined by complementarity-determining regions or CDRs, such as light chain CDRs and/or heavy chain CDRs. In some embodiments, the binding specificity of the extracellular antigen-binding region of a CAR may be determined by the light chain variable region and/or heavy chain variable region.

In some embodiments, the extracellular region of a CAR comprises a hinge or spacer region, wherein the hinge and spacer regions may be used interchangeably. The hinge may be considered as a part of the CAR that provides flexibility to the extracellular antigen-binding region. In some embodiments, the hinge may be used to detect CARs on the cell surface, particularly when an antibody detecting the extracellular antigen-binding region are ineffective or unavailable. In some embodiments, the hinge may not belong to an immunoglobulin but to another molecule, such as the natural hinge of the CD8α molecule. The CD8α hinge may comprise cysteine and proline residues known to play a role in the interaction between the CD8 co-receptor and MHC molecules. The hinge may be adjusted based on the used extracellular antigen-binding region. The hinge may be of any length. In some embodiments, the hinge may be the natural hinge region of IgG1 or IgG4, or a mutated hinge region thereof, or other structural parts beyond the hinge region. For example, the hinge may comprise an amino acid sequence as shown in SEQ ID NO: 21, 27, 28, 29, or 30.

The transmembrane domain (or region) of the CAR anchors the CAR in the plasma membrane of the cell. The natural transmembrane portion of CD28 may be used in the CAR. In other cases, the natural transmembrane portion of CD8α may also be used in the CAR. “CD8” may refer to a protein with at least 85%, 90%, 95%, 96%, 97%, 98%, 99%, or 100% identity to the NCBI reference number: NP_001759 or a fragment thereof with a stimulatory activity. A “CD8 nucleic acid molecule” may be a polynucleotide encoding a CD8 polypeptide. In certain cases, the transmembrane region may be the natural transmembrane portion of CD28. “CD28” may refer to a protein with at least 85%, 90%, 95%, 96%, 97%, 98%, 99%, or 100% identity to the NCBI reference number: NP_006130 or a fragment thereof with a stimulatory activity. A “CD28 nucleic acid molecule” may be a polynucleotide encoding a CD28 polypeptide. In some embodiments, the transmembrane portion may comprise a CD8α region. For example, the transmembrane domain may comprise an amino acid sequence as shown in SEQ ID NO: 22 or 25.

The intracellular signaling region of the CAR is responsible for activating at least one effector function of the immune response cell comprising the CAR. The CAR may induce effector functions of T cells, such as cytolytic activity or helper activity, comprising the secretion of cytokines like IL-2, TNF-α, γ-IFN, etc. Therefore, the term “intracellular signaling domain” refers to the portion of a protein that transduces effector function signals and directs the cell to perform specific functions. While the entire intracellular signaling region is typically used, in many cases, it is not necessary to use the entire chain of the signaling domain. In some embodiments, a truncated portion of the intracellular signaling region is used. Therefore, the term “intracellular signaling region” is intended to comprise any truncated portion of the intracellular signaling region sufficient to transduce effector function signals.

Preferred examples of signaling domains (or regions) used in CARs comprise cytoplasmic sequences of the T cell receptor (TCR) and co-receptors that act together to initiate signal transduction upon target-receptor binding, as well as any derivatives or variant sequences thereof and any synthetic sequences with the same functionality.

An example of a T cell signaling domain comprising one or more ITAM motifs is the CD3 ζ domain, also known as the T cell receptor CD3ζ chain or CD247. This domain is a part of the T cell receptor-CD3 complex and plays a crucial role in coupling the antigen recognition of several intracellular signal transduction pathways with the primary effector activation of T cells. As used herein, CD3ζ primarily refers to human CD3ζ and isoforms thereof, such as those known from the SwissProt entry P20963, comprising proteins with substantially identical sequences. As a part of a chimeric antigen receptor, the entire T cell receptor CD3ζ chain is not required, and any derivative thereof comprising the signaling domain of the T cell receptor CD3ζ chain is suitable, comprising any functional equivalents. For example, the signaling domain of the CD3ζ chain may comprise an amino acid sequence as shown in SEQ ID NO: 24.

In certain embodiments, the intracellular signaling domain (or region) of the CAR may be selected from any of the co-stimulatory domains listed in Table 2. In some embodiments, the domain may be modified so that it can have about 50% to about 100% identity to the reference domain. Any of the domains in Table 1 may be modified, and the modified form may contain about 50%, 60%, 70%, 80%, 90%, 95%, 96%, 97%, 98%, 99%, or up to about 100% identity.

In certain embodiments, the intracellular signaling region of the CAR may further comprise one or more co-stimulatory domains. The intracellular signaling region may comprise a single co-stimulatory domain, such as the ζ chain (first-generation of CAR) or in combination with CD28 or 4-1BB (second-generation of CAR). In other embodiments, the intracellular signaling region may comprise two co-stimulatory domains, such as CD28/OX40 or CD28/4-1BB (third-generation). For example, the co-stimulatory domain of CD28 may comprise an amino acid sequence as shown in SEQ ID NO: 23.

In certain embodiments, these co-stimulatory domains can, together with intracellular signaling domains such as CD8, induce downstream activation of kinase pathways, thereby supporting the gene transcription and functional cellular responses. The co-stimulatory domain of the CAR may activate proximal signaling proteins associated with the CD28 (phosphatidylinositol-4,5-bisphosphate 3-kinase) or 4-1BB/OX40 (TNF receptor-associated factor adaptor protein) pathways, as well as MAPK and Akt activation.

In certain cases, signals generated by the CAR may be combined with auxiliary or co-stimulatory signals. For co-stimulatory signaling domains, chimeric antigen receptor-like complexes may be designed to comprise several possible co-stimulatory signaling domains. As is well-known in the art, in naive T cells, binding of the T cell receptor alone is insufficient to induce complete activation of T cells into cytotoxic T cells. The complete activation of productive T cells requires a second co-stimulatory signal. Several receptors that provide a co-stimulation for T cell activation have been reported, comprising but not limited to CD28, OX40, CD27, CD2, CD5, ICAM-1, LFA-1 (CD11a/CD18), 4-1BBL, MyD88, and 4-1BB. The signaling pathways used by these co-stimulatory molecules can synergize with the primary T cell receptor activation signal. The signals provided by these co-stimulatory signaling regions can synergize with the primary effector activation signal derived from one or more ITAM motifs (e.g., the CD3ζ signal transduction domain) and fulfill the requirements for T cell activation.

In some embodiments, adding a co-stimulatory domain to the chimeric antigen receptor-like complex can enhance the efficacy and durability of engineered cells. In other embodiments, the T cell signaling domain and co-stimulatory domain are fused to each other to constitute the signaling region.

TABLE 2
Co-stimulatory Domains

| Gene | | |
|---|---|---|
| Symbol | Abbreviation | Name |
| CD27 | CD27; T14; S152; Tp55; | CD27 molecule |
| | TNFRSF7; S152. LPFS2 | |
| CD28 | Tp44; CD28; CD28 antigen | CD28 molecule |
| TNFRSF9 | ILA; 4-1BB; CD137; | Tumor necrosis factor |
| | CDw137 | receptor superfamily |
| | | member 9 |
| TNFRSF4 | OX40; ACT35; CD134; | Tumor necrosis factor |
| | IMD16; TXGP1L | receptor superfamily |
| | | member 4 |
| TNFRSF8 | CD30; Ki-1; D1S166E | Tumor necrosis factor |
| | | receptor superfamily |
| | | member 8 |
| CD40LG | IGM; IMD3; TRAP; gp39; | CD40 ligand |
| | CD154; CD40L; HIGM1; | |
| | T-BAM; TNFSF5; hCD40L | |
| ICOS | AILIM; CD278; CVID1 | Inducible T cell |
| | | co-stimulator |
| ITGB2 | LAD; CD18; MF17; MFI7; | Integrin β2 |
| | LCAMB; LFA-1; MAC-1 | (complement |
| | | component 3 receptor |
| | | 3 and 4 subunits) |
| CD2 | T11; SRBC; LFA-2 | CD2 molecule |
| CD7 | GP40; TP41; Tp40; LEU-9 | CD7 molecule |
| KLRC2 | NKG2C; CD159c; NKG2-C | Killer cell lectin-like |
| | | receptor subfamily C, |
| | | member 2 |
| TNFRSF18 | AITR; GITR; CD357; | Tumor necrosis factor |
| | GITR-D | receptor superfamily |
| | | member 18 |
| TNFRSF14 | TR2; ATAR; HVEA; | Tumor necrosis factor |
| | HVEM; CD270; LIGHTR | receptor superfamily |
| | | member 14 |
| HAVCR1 | TIM; KIM1; TIM1; CD365; | Hepatitis A virus |
| | HAVCR; KIM-1; TIM-1; | cellular receptor 1 |
| | TIMD1; TIMD-1; HAVCR-1 | |
| LGALS9 | HUAT; LGALS9A, | Lectin, |
| | Galectin-9 | galactoside-binding, |
| | | soluble, 9 |
| CD83 | BL11; HB15 | CD83 molecule |

The present invention provides cells (e.g., T cells) engineered to express CARs, wherein these CAR-expressing cells (e.g., CAR-T cells) exhibit anti-tumor properties. In one aspect, cells are transduced with a CAR, which is expressed on the cell surface. In some embodiments, cells (e.g., T cells) are transduced with a viral vector encoding the CAR. In some embodiments, the viral vector is a retroviral vector. In some embodiments, the viral vector is a lentiviral vector. In some embodiments, cells may stably express the CAR. In some embodiments, cells (e.g., T cells) are transfected with nucleic acids encoding the CAR, such as mRNA, cDNA, or DNA.

In certain embodiments, the CAR comprises an antigen-binding domain targeting CLDN6. In certain embodiments, the CLDN6-binding portion of the CAR is an scFv, which is functional and retains binding affinity comparable to the IgG antibody from which it is derived, for example, binding the antigen with comparable efficacy; thereby providing a biochemical reaction, such as activating an immune response, inhibiting signal initiation from its target antigen, inhibiting kinase activity, etc. For example, the anti-CLDN6 antigen-binding domain of the CAR comprises an scFv sequence as shown in any one of SEQ ID NO: 13, 14, 15, 16, 17, 18, or 19.

In certain embodiments, the anti-CLDN6 antigen-binding domain of the CAR is a humanized antibody or fragment thereof. In certain embodiments, the anti-CLDN6 antigen-binding domain of the CAR is a fully human antibody or fragment thereof. In certain embodiments, the anti-CLDN6 antigen-binding domain of the CAR is a murine antibody or fragment thereof.

In certain embodiments, in the CAR of the present invention, the antigen-binding domain of a specific antibody is combined with an intracellular signaling molecule. For example, the intracellular signaling molecule comprises but is not limited to CD3ζ chain, 4-1BB, and CD28 signaling modules and combinations thereof.

In certain embodiments, the CLDN6-CAR comprises at least one intracellular signaling domain selected from a CD137 (4-1BB) signaling domain, CD28 signaling domain, CD3ζ signaling domain, and any combination thereof. In one aspect, the CLDN6-CAR comprise at least one intracellular signaling domain derived from one or more co-stimulatory molecules other than CD137 (4-1BB) or CD28.

As an example, the sequence of the CLDN6-CAR comprises: an extracellular domain as shown in SEQ ID NO: 13, a hinge domain as shown in SEQ ID NO: 21, a transmembrane domain as shown in SEQ ID NO: 22, a co-stimulatory signaling domain as shown in SEQ ID NO: 23, and a primary signaling domain as shown in SEQ ID NO: 24 (H1-28Z); or an extracellular domain as shown in SEQ ID NO: 17, a hinge domain as shown in SEQ ID NO: 21, a transmembrane domain as shown in SEQ ID NO: 22, a co-stimulatory signaling domain as shown in SEQ ID NO: 23, and a primary signaling domain as shown in SEQ ID NO: 24 (P4-28Z).

Exemplarily, the amino acid sequence of the chimeric antigen receptor comprises the sequence shown in any one of SEQ ID NO: 13, 14, 15, 16, 17, 18, or 19 connected to the sequence shown in any one of SEQ ID NO: 45, 46, or 47. For example, the amino acid sequence of the chimeric antigen receptor comprises: the sequence shown in SEQ ID NO: 13 linked to the sequence shown in SEQ ID NO: 45; or the sequence shown in SEQ ID NO: 13 linked to the sequence shown in SEQ ID NO: 46; or the sequence shown in SEQ ID NO: 13 linked to the sequence shown in SEQ ID NO: 47; or the sequence shown in SEQ ID NO: 14 linked to the sequence shown in SEQ ID NO: 45; or the sequence shown in SEQ ID NO: 14 linked to the sequence shown in SEQ ID NO: 46; or the sequence shown in SEQ ID NO: 14 linked to the sequence shown in SEQ ID NO: 47; or the sequence shown in SEQ ID NO: 15 linked to the sequence shown in SEQ ID NO: 45; or the sequence shown in SEQ ID NO: 15 linked to the sequence shown in SEQ ID NO: 46; or the sequence shown in SEQ ID NO: 15 linked to the sequence shown in SEQ ID NO: 47; or the sequence shown in SEQ ID NO: 16 linked to the sequence shown in SEQ ID NO: 45; or the sequence shown in SEQ ID NO: 16 linked to the sequence shown in SEQ ID NO: 46; or the sequence shown in SEQ ID NO: 16 linked to the sequence shown in SEQ ID NO: 47; or the sequence shown in SEQ ID NO: 17 linked to the sequence shown in SEQ ID NO: 45; or the sequence shown in SEQ ID NO: 17 linked to the sequence shown in SEQ ID NO: 46; or the sequence shown in SEQ ID NO: 17 linked to the sequence shown in SEQ ID NO: 47; or the sequence shown in SEQ ID NO: 18 linked to the sequence shown in SEQ ID NO: 45; or the sequence shown in SEQ ID NO: 18 linked to the sequence shown in SEQ ID NO: 46; or the sequence shown in SEQ ID NO: 18 linked to the sequence shown in SEQ ID NO: 47; or the sequence shown in SEQ ID NO: 19 linked to the sequence shown in SEQ ID NO: 45; or the sequence shown in SEQ ID NO: 19 linked to the sequence shown in SEQ ID NO: 46; or the sequence shown in SEQ ID NO: 19 linked to the sequence shown in SEQ ID NO: 47.

For the transmembrane and intracellular domains of the above chimeric antigen receptors, a skilled person may choose conventional transmembrane and intracellular domains for replacement, all of which fall within the scope of protection of the present application.

### Nucleic Acid, Vector, Viruse, Host Cell

The terms “nucleic acid” or “polynucleotide” refer to deoxyribonucleic acid (DNA) or ribonucleic acid (RNA) in a single-stranded or double-stranded form and polymers, oligonucleotides thereof, fragments generated by PCR, fragments generated by any one of ligation, cleavage, endonuclease action, and exonuclease action. Nucleic acid sequences comprise natural nucleotide sequences and implicitly comprise conservatively modified variants thereof (e.g., degenerate codon substitutions), alleles, orthologs, SNPs, and complementary sequences. Modified nucleotides may have alterations in the sugar moiety and/or pyrimidine or purine base moiety. For example, sugar modifications comprise replacing one or more hydroxyl groups with halogens, alkyl groups, amines, and azido groups, or the sugar may be functionalized with ether or ester groups.

The term “codon” refers to three nucleotides on mRNA (or on the sense strand of a DNA molecule) that is translated into an amino acid residue by ribosomes.

The term “codon optimization” refers to the preference of the frequency of synonymous codons (codons encoding the same amino acid) occurring in coding DNA among different species. Rare codons in highly expressed genes of a given species are replaced by common codons from highly expressed genes of that species. The codon degeneracy allows multiple nucleotide sequences to encode the same polypeptide. The degenerate codon substitution can be achieved by creating a sequence where one or more selected (or all) codons have the third position replaced with mixed bases and/or deoxyinosine residues.

The term “encoding” refers to inherent properties of a specific nucleotide sequence in a polynucleotide, such as a gene, DNA, or mRNA, to serve, in a biological process, as a template for the synthesis of other polymers and macromolecules with defined nucleotide sequences (e.g., rRNA, tRNA, and mRNA) or defined amino acid sequences and the resulting biological properties. Therefore, when the mRNA corresponding to a gene is transcribed and translated in a cell or other biological system to produce a protein, the gene, cDNA, or RNA encodes that protein. Both the coding strand (the nucleotide sequence of which is identical to the mRNA sequence and is usually provided in sequence listings) and the non-coding strand (used as a template for the transcription of a gene or cDNA) can be referred to as encoding the protein or other products of a gene or cDNA.

The terms “nucleic acid molecule encoding”, “coding DNA sequence”, and “coding DNA” refer to the order or sequence of deoxyribonucleotides along a deoxyribonucleic acid strand. The order of these deoxyribonucleotides determines the order of amino acids along a polypeptide (protein) chain. Therefore, a nucleic acid sequence encodes an amino acid sequence. A “nucleotide sequence encoding an amino acid sequence” comprises all nucleotide sequences that are degenerate forms of each other and encode the same amino acid sequence. When used to refer to a nucleotide sequence, the term “sequence” as used herein comprises DNA or RNA and may be single-stranded or double-stranded.

The term “target sequence” refers to a sequence complementary to the guide sequence. The complementary pairing between the target sequence and the guide sequence facilitates the formation of the CRISPR complex. A target sequence can comprise any polynucleotide, such as DNA or RNA polynucleotides. In some embodiments, the target sequence is located in the nucleus or cytoplasm of a cell.

The term “identity” or “homology” refers to the subunit sequence identity between two nucleic acid molecules or two polypeptide molecules. When a subunit position is occupied by the same monomeric subunit in both molecules (for example, when both DNA molecules are occupied by adenosine at a given position), they are considered homologous or identical at that position. The homology between two sequences is calculated as the number of matched or homologous positions divided by the total number of positions in the sequence, and multiplied by 100 to obtain the percentage of sequence identity. For example, if 80% of the positions in two sequences (e.g., 8 out of 10 base pairs in a 10-bp nucleic acid molecule) are homologous, the homology between the two sequences is 80%.

The terms “transfection”, “transduction”, or “transformation” refer to the introduction of exogenous nucleic acids into a host cell. The transfection can be achieved through various methods known in the art, comprising calcium phosphate-DNA co-precipitation, DEAE-dextran-mediated transfection, polybrene-mediated transfection, electroporation, microinjection, liposome fusion, lipofection, protoplast fusion, retroviral infection, and biolistics. A “transfected”, “transformed” or “transduced” cell is a cell that has been transfected, transformed, or transduced with exogenous nucleic acids. Such cell comprises a primary subject cell and progenies thereof.

The term “expression vector” refers to a vector comprising a recombinant polynucleotide which comprises an expression regulatory sequence operably linked to the nucleotide sequence to be expressed. An expression vector comprises sufficient cis-acting elements for expression; and other elements required for expression may be provided by the host cell or an in vitro expression system. Expression vectors comprise all those known in the art, such as plasmids and viruses (e.g., lentiviruses, retroviruses, adenoviruses, and adeno-associated viruses). The plasmid vector may also comprise a selectable marker that enables the identification and/or screening of cells receiving the vector.

The term “vector” refers to a composition which comprises an isolated nucleic acid and can be used to deliver the isolated nucleic acid into a cell. Numerous vectors are known in the art, comprising but not limited to linear polynucleotides, polynucleotides associated with ions or amphiphilic compounds, plasmids, and viruses. Therefore, the term “vector” encompasses autonomously replicating plasmids or viruses. It may also comprise non-plasmid and non-viral compounds that facilitate the transfer of a nucleic acid into cells, such as polylysine compounds, liposomes, etc. Vectors can be associated or combined with any cell permeabilization technique, such as sonoporation, electroporation, or derivative techniques thereof. The choice of a vector depends primarily on the size of the nucleic acid to be inserted and the specific host cell to be transfected. Each vector comprises different components based on its function (amplification and/or expression of heterologous polynucleotides) and compatibility with the specific host cell in which it presents. Vector components typically comprise, but are not limited to: an origin of replication, a selectable marker gene, a promoter, a ribosome-binding site (RBS), a signal sequence, a heterologous nucleic acid insert, and a transcription termination sequence. Physical methods for introducing vectors into cells comprise calcium phosphate precipitation, liposome transfection, particle bombardment, microinjection, electroporation, etc. Chemical methods for introducing vectors into cells comprise colloidal dispersion systems, such as macromolecular complexes, nanocapsules, microspheres, beads, and lipid-based systems (comprising oil-in-water emulsions, micelles, mixed micelles, and liposomes).

The term “lentivirus” refers to a genus of the Retroviridae family. Lentiviruses are unique among retroviruses in their ability to infect non-dividing cells. They can deliver a large amount of genetic information into the DNA of a host cell, and thus, deemed as the most efficient methods. Examples of lentiviruses comprise HIV, SIV, and FIV. Vectors derived from lentiviruses provide a means to achieve significant levels of gene transfer in vivo. “Integrating lentiviral vectors (LV)” are non-limiting examples of such vectors, which can be integrated into the genome of target cells. In contrast, “non-integrating lentiviral vectors (NILVs)” refer to vectors for efficient gene delivery which are not integrated into the genome of target cells via the action of viral integrase.

The term “endogenous” refers to any substance originating from or produced within an organism, cell, tissue, or system itself, such as nucleic acid molecules or polypeptides.

The term “exogenous” refers to any substance introduced into or produced outside an organism, cell, tissue, or system, such as nucleic acid molecules, polypeptides, or cells.

An “exogenous protein” may be a protein introduced into cells exogenously to recognize a target antigen, such as an exogenous receptor (i.e., the “chimeric receptor” aforementioned herein).

The term “host” refers to a recipient receiving a graft transplant. In some embodiments, it may be an individual receiving exogenous cell implantation, such as a human.

The term “isolated” refers to being altered or removed from its natural state. For example, a nucleic acid or peptide naturally present in a living animal is not “isolated”, however the same nucleic acid or peptide partially or completely separated from its naturally existing substances is “isolated”. Isolated nucleic acids or proteins may exist in a substantially pure form or in a non-natural environment, such as within a host cell. An “isolated” substance can also be provided through artificial assembly methods, such as chemical synthesis or recombinant expression.

The term “expression” refers to the transcription and/or translation of a specific nucleotide sequence driven by a promoter.

The term “promoter” is a DNA sequence recognized, bound, and initiated for transcription by RNA polymerase, which is a critical component of a gene, primarily functioning to regulate the timing and degree of gene transcription. In some embodiments, the nucleic acid encoding a CAR may be operably linked to a promoter.

Isolated nucleic acids encoding antibodies or fragments thereof that recognize CLDN6, vectors, and host cells comprising such nucleic acids or vectors are provided in the present invention. The nucleic acid may be located within an intact cell, cell lysate, or in a partially purified or substantially purified form.

Standard molecular biology techniques can be used to obtain the nucleic acids of the invention. For example, cDNAs encoding the light and heavy chains of an antibody or the VH and VL regions can be obtained via a standard PCR amplification or cDNA cloning techniques. For antibodies obtained from immunoglobulin gene libraries (e.g., using phage display technology), one or more nucleic acids encoding the antibody can be recovered from the library. Methods for introducing exogenous nucleic acids into host cells are well-known in the art and may vary depending on the used host cell.

Preferably, the nucleic acid molecules of the invention are selected from SEQ ID NO: 2 or 6 encoding the heavy chain variable region and/or from SEQ ID NO: 4, 8, 10, or 12 encoding the light chain variable region. More preferably, the nucleic acid molecules comprise the heavy chain variable region sequence of SEQ ID NO: 2 and the light chain variable region sequence of SEQ ID NO: 4; or the heavy chain variable region sequence of SEQ ID NO: 2 and the light chain variable region sequence of SEQ ID NO: 8; or the heavy chain variable region sequence of SEQ ID NO: 2 and the light chain variable region sequence of SEQ ID NO: 10; or the heavy chain variable region sequence of SEQ ID NO: 6 and the light chain variable region sequence of SEQ ID NO: 4; or the heavy chain variable region sequence of SEQ ID NO: 6 and the light chain variable region sequence of SEQ ID NO: 8; or the heavy chain variable region sequence of SEQ ID NO: 6 and the light chain variable region sequence of SEQ ID NO: 10; or the heavy chain variable region sequence of SEQ ID NO: 2 and the light chain variable region sequence of SEQ ID NO: 12. In one embodiment, one or more vectors (e.g., expression vectors) comprising the aforementioned nucleic acids are provided.

The term “cell” refers to cells derived from human or non-human animals.

The term “host cell” refers to a cell into which exogenous nucleic acids have been introduced, comprising progenies of the cell. Host cells comprise “transformants” and “transformed cells”, which encompass transformed primary cells and progenies thereof (regardless of the number of passages). The contents of nucleic acids of the progeny may not be identical to those of the parental cell and may comprise mutations. Mutant progenies with the same functional or biological activity as screened or selected in the original transformed cell are comprised herein.

The term “positive” for a cell or cell population with respect to a specific marker refers to the detectable presence of the specific marker (typically a surface marker) on or within the cell. When referring to a surface marker, the term indicates that the surface expression is detected via flow cytometry, for example, by staining with an antibody specifically binding to the marker and detecting the antibody, wherein the staining is detectable by flow cytometry at a level significantly higher than that observed under identical conditions using an isotype-matched control, and/or substantially similar to the level observed in cells known to be positive for the marker, and/or significantly higher than the level observed in cells known to be negative for the marker.

The term “negative” for a cell or cell population with respect to a specific marker refers to the essentially undetectable presence of the specific marker (typically a surface marker) on or within the cell. When referring to a surface marker, the term indicates that the surface expression is not detected via flow cytometry, for example, by staining with an antibody specifically binding to the marker and detecting the antibody, wherein the staining is detectable by flow cytometry at a level significantly lower than that observed under identical conditions using an isotype-matched control, and/or significantly lower than the level observed in cells known to be positive for the marker, and/or substantially similar to the level observed in cells known to be negative for the marker.

The term “CLDN6-positive host cell” refers to a host cell that expresses CLDN6 on the surface. Such cells can be detected by, for example, using antibodies specifically recognizing epitopes on CLDN6 via flow cytometry.

In some embodiments, the host cell is an immune cell.

The term “immune cell” refers to a cell involved in immune responses or producing immune effects, such as a T cell, B cell, natural killer (NK) cell, natural killer T (NKT) cell, dendritic cell, CIK cell, macrophage, mast cell, neutrophil, eosinophil, and/or basophil. In some embodiments, the immune cell is a T cell, NK cell, or NKT cell. In some embodiments, the T cell may be an autologous T cell, xenogeneic T cell, or allogeneic T cell. In some embodiments, the NK cell may be an allogeneic NK cell. “Immune effector function or immune effector response” refers to the function or reaction of immune cells that, for example, enhances or promotes an immune attack on target cells. For example, an immune function or response refers to the property of a T cell or NK cell that promotes the killing of target cells or inhibits their growth or proliferation.

The term “artificially engineered cell with immune cell function” refers to a cell or cell line which lacks immune effector capabilities however, through artificial engineering or stimulation, acquires immune cell functions. For example, 293T cells can be artificially engineered to acquire immune cell functions, or stem cells can be induced in vitro to differentiate into immune cells. Stem cells may be adult stem cells, non-human embryonic stem cells, more specifically non-human stem cells, umbilical cord blood stem cells, progenitor cells, bone marrow stem cells, induced pluripotent stem cells, totipotent stem cells, or hematopoietic stem cells.

In some cases, the cells are generally primary cells, such as those directly isolated from an individual or isolated from an individual and frozen. In some embodiments, the cells comprise one or more subsets of T cells or other cell types, such as the entire T cell population, CD4± cells, CD8+ cells, and subpopulations, as defined by function, activation state, maturity, differentiation potential, expansion, recirculation, localization, and/or persistence capabilities, antigen specificity, antigen receptor type, presence in specific organs or compartments, marker or cytokine secretion profiles, and/or degree of differentiation. For the individual to be treated, the cells may be allogeneic and/or autologous.

In some cases, “T cells” may be multipotent stem cells derived from the bone marrow that differentiate and mature in the thymus to become immunologically active mature T cells. In some cases, “T cells” may be a cell population with specific phenotypic characteristics or a mixed population of cells with different phenotypic characteristics. For example, “T cells” may cells comprising at least one T cell subpopulation: stem cell-like memory T cells (Tscm cells), central memory T cells (Tcm), effector T cells (Tef or Teff), regulatory T cells (Tregs), effector memory T cells (Tem), naive T cells (TN), tumor-infiltrating lymphocytes (TILs), immature T cells, mature T cells, helper T cells (e.g., TH1, TH2, TH3, TH17, TH9, TH22, follicular helper T cells), cytotoxic T cells, and/or mucosal-associated invariant T (MAIT) cells. In some cases, “T cells” may be a specific subtype of T cells, such as β T cells or γô T cells.

T cells can be obtained from many sources, comprising PBMCs, bone marrow, lymph node tissue, umbilical cord blood, thymus tissue, and tissues from infection sites, ascites, pleural effusions, spleen tissue, and tumors. In certain cases, T cells can be obtained from blood collected from an individual using any technique known to a skilled person, such as Ficoll™ separation. In one embodiment, cells from circulating blood are obtained from an individual via apheresis. Apheresis products typically contain lymphocytes, comprising T cells, monocytes, granulocytes, B cells, other nucleated white blood cells, red blood cells, and platelets. In one embodiment, cells collected via apheresis are washed to remove plasma molecules and placed in a suitable buffer or medium for subsequent processing steps. In one embodiment, cells may be derived from healthy donors or from patients diagnosed with tumors. CAR-T cells can be prepared using conventional methods in the art, such as by activating PBMCs with anti-CD3 and CD28 antibody-coated magnetic beads, followed by culture and lentiviral infection.

The term “peripheral blood mononuclear cell (PBMC)” refers to mononuclear cells in peripheral blood, comprising lymphocytes, monocytes, etc.

The terms “activation” and “activating” are used interchangeably and may refer to the process by which a cell transitions from a resting state to an active state. The process may comprise responses to antigens, phenotypic or genetic changes in migration, and/or functional activity states. For example, the term “activation” may refer to a stepwise activation process of NK cells or T cells.

The term “T cell activation” or “T cell activating” refers to the state of T cells that are sufficiently stimulated to induce detectable cell proliferation, cytokine production, and/or detectable effector functions.

“Genetic engineering” generally involves introducing nucleic acids encoding the recombinant or engineered components into cells, for example, via retroviral or lentiviral transduction, transfection, or transformation, or via transposons or electroporation. In some embodiments, this is achieved by first stimulating the cells, for example, by combining them with a stimulant that induces a response, such as proliferation, survival, and/or activation, as detected by cytokine or activation marker expression, and then transducing the activated cells and expanding them in a culture to a quantity sufficient for clinical application.

In another embodiment, a host cell comprising the aforementioned nucleic acid is provided. The host cell comprises (e.g., transduced with): (1) a vector comprising a nucleic acid encoding an amino acid sequence comprising an antibody VL and an amino acid sequence comprising an antibody VH, or (2) a first vector comprising a nucleic acid encoding an amino acid sequence comprising an antibody VL and a second vector comprising a nucleic acid encoding an amino acid sequence comprising an antibody VH. In one embodiment, the host cell is eukaryotic, such as a 293T cell.

In another embodiment, the host cell expresses the chimeric receptor described herein.

In another embodiment, the host cell comprises a T cell, natural killer cell, cytotoxic T lymphocyte, natural killer T cell, DNT cell, regulatory T cell, NK92 cell, and/or stem cell-derived immune cell.

In another embodiment, the T cell is derived from a natural T cell and/or induced from a pluripotent stem cell. Preferably, the T cell is an autologous/allogeneic T cell; preferably, the T cell is a primary T cell; and preferably, the T cell is derived from a human autologous T cell.

In another embodiment, the T cell comprises a stem cell-like memory T cell (Tscm cell), central memory T cell (Tcm), effector T cell (Tef), regulatory T cell (Treg), effector memory T cell (Tem), γδ T cell, or combinations thereof.

In another embodiment, the host cell binds to a cell expressing CLDN6 but does not significantly bind to a cell expressing CLDN4, CLDN9, or combinations thereof.

In another embodiment, the host cell also carries an exogenous coding sequence for a cytokine.

In another embodiment, the host cell may also express another chimeric receptor in addition to the aforementioned antigen-binding receptor.

In another embodiment, the host cell may also express a chemokine receptor.

In another embodiment, the host cell may also express a safety switch.

In one embodiment, a method for producing an anti-CLDN6 antibody is provided, wherein the method comprises culturing a host cell comprising a nucleic acid encoding the antibody under conditions suitable for expressing the aforementioned antibody and optionally recovering the antibody from the host cell (or host cell medium).

To express a protein, the nucleic acid encoding the antibody of the invention can be integrated into an expression vector. A variety of expression vectors can be used for expressing the protein. Expression vectors may comprise self-replicating extrachromosomal vectors or vectors integrated into the host genome. Expression vectors which can be used in the invention comprise, but are not limited to, those that enable the protein expression in mammalian cells, bacteria, insect cells, yeast, and in vitro systems. As known in the art, many expression vectors are commercially or otherwise available, which can be used in the invention to express antibodies.

In a preferred example, the host cell is administered in combination with an agent that enhances its function, preferably in combination with a chemotherapeutic agent; and/or the host cell is administered in combination with an agent that ameliorates one or more related side effects; and/or the host cell is administered in combination with a host cell expressing a chimeric antigen receptor targeting something other than CLDN6.

### Pharmaceutical Compositions and Combination Therapies

The antibodies of the invention, immunoconjugates, chimeric receptors, and host cells comprising the antibodies can be used to prepare pharmaceutical compositions or diagnostic reagents. The composition may comprise, in addition to an effective amount of the antibody, an immunoconjugate, chimeric receptor, nucleic acid, or host cell, a pharmaceutically acceptable carrier.

The term “pharmaceutically acceptable” means that when the molecular entity and composition are properly administered to an animal or human, they do not produce adverse, allergic, or other undesirable reactions. Specific examples of substances that may be used as pharmaceutically acceptable carriers or components thereof comprise sugars, such as lactose, glucose, mannose, sucrose, and dextran; starches, such as corn starch and potato starch; cellulose and its derivatives, such as sodium carboxymethylcellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil, and cocoa oil; polyols, such as propylene glycol, glycerol, sorbitol, mannitol, and polyethylene glycol; alginic acid; emulsifiers, such as Tween; wetting agents, such as sodium lauryl sulfate; coloring agents; flavoring agents; tableting agents; stabilizers; antioxidants; preservatives; pyrogen-free water; isotonic saline; phosphate buffers; adjuvants, such as aluminum hydroxide; buffers; diluents; stabilizers; or excipients, and the like.

The pharmaceutical compositions described herein may comprise one or more pharmaceutically acceptable salts. A “pharmaceutically acceptable salt” refers to a salt that retains the desired biological activity of the parent compound and does not produce any undesirable toxicological effects. Examples of such salts comprise acid addition salts and base addition salts.

Acid addition salts comprise those derived from nontoxic inorganic acids, such as hydrochloric, nitric, phosphoric, sulfuric, hydrobromic, hydroiodic, phosphorous acids, and the like, as well as those derived from nontoxic organic acids, such as aliphatic mono- and dicarboxylic acids, phenyl-substituted alkanoic acids, hydroxyalkanoic acids, aromatic acids, aliphatic and aromatic sulfonic acids, and the like. Base addition salts comprise those derived from alkaline earth metals, such as sodium, potassium, magnesium, calcium, and the like, as well as salts derived from nontoxic organic amines, such as N,N′-dibenzylethylenediamine, N-methylglucamine, chloroprocaine, choline, diethanolamine, ethylenediamine, procaine, and the like.

The pharmaceutical compositions described herein may also comprise antioxidants. Examples of antioxidants comprise, but are not limited to: water-soluble antioxidants, such as ascorbic acid, cysteine hydrochloride, sodium bisulfite, sodium metabisulfite, sodium sulfite, and the like; oil-soluble antioxidants, such as ascorbyl palmitate, butylated hydroxyanisole (BHA), butylated hydroxytoluene (BHT), lecithin, propyl gallate, α-tocopherol, and the like; and metal chelators, such as citric acid, ethylenediaminetetraacetic acid (EDTA), sorbitol, tartaric acid, phosphoric acid, and the like.

The compositions of the invention can be formulated into various dosage forms as needed and administered by a physician based on factors, such as the patient's species, age, weight, and general disease condition, as well as the route of administration. Administration manners may be, for example, parenteral administration (e.g., injection) or other therapeutic approaches. “Parenteral” administration of immunogenic compositions comprises, for example, subcutaneous (s.c.), intravenous (i.v.), intramuscular (i.m.), or intrasternal injection or infusion techniques. A patient can be administered via an intra-arterial, intradermal, intratumoral, intranodal, intramedullary, or intraperitoneal route.

In some embodiments, the compositions may be isotonic, that is, they may have the same osmotic pressure as blood and tears. The desired isotonicity of the composition of the invention can be achieved using sodium chloride or other pharmaceutically acceptable agents, such as glucose, boric acid, sodium tartrate, propylene glycol, or other inorganic or organic solutes. If necessary, the viscosity of the composition can be maintained at a selected level by using a pharmaceutically acceptable thickening agent. Suitable thickening agents comprise, for example, methylcellulose, xanthan gum, carboxymethylcellulose, hydroxypropyl cellulose, carbomer, and the like. The preferred concentration of the thickening agent will depend on the selected agent. The choice of suitable carriers and other additives will obviously depend on the exact route of administration and the nature of the particular dosage form, such as a liquid dosage form.

In one embodiment, the anti-CLDN6 antibodies, immunoconjugates, host cells modified with chimeric receptors, pharmaceutical compositions, or kits of the invention are administered in combination with other known active agents or therapeutic treatments. “Administration in combination” refers to the administration of two or more different treatments to an individual. In one embodiment, one treatment is still ongoing when the second treatment begins, thereby resulting in an overlap in the administration. In one embodiment, the second treatment begins after the first treatment ended. In one embodiment, the administration in combination enhances the efficacy of the treatment. In some embodiments, the effects of the two treatments may be partially additive, fully additive, or greater than additive. The treatments administered in combination comprise, but are not limited to, one or more of the following: surgery, chemotherapy, radiation, immunosuppressants (e.g., cyclosporine, azathioprine, methotrexate, mycophenolate, and FK506), antibodies or other immunoablative agents, such as CAMPATH, anti-CD3 antibodies or other antibody therapies, cyclophosphamide, fludarabine, rapamycin, mycophenolic acid, steroids, FR901228, cytokines, and radiation. The treatments administered in combination also comprise the administration of immunomodulators, such as interferon-α, interferon-β, TGF-β2 peptide inhibitors, or poly-ICLC.

In some embodiments, the composition comprises another therapeutic agent. In some embodiments, the anti-CLDN6 antibodies, immunoconjugates, host cells modified with chimeric receptors, pharmaceutical compositions, or kits of the invention are administered in combination with an agent that enhances their function. In some embodiments, the other therapeutic agent is a chemotherapeutic agent, such as those described in US20140271820 and/or a pharmaceutically acceptable salt or analog thereof. Examples of chemotherapeutic agents comprise alkylating agents, platinum-based agents, angiogenesis inhibitors (e.g., VEGF pathway inhibitors, tyrosine kinase inhibitors, EGF pathway inhibitors), and mTOR inhibitors. In some embodiments, the therapeutic agents comprise, but are not limited to, mitotic inhibitors (vinca alkaloids), comprising vincristine, vinblastine, vindesine, and Navelbine™ (vinorelbine, 5′-dehydrodesacetylvinblastine); topoisomerase I inhibitors, such as camptothecin compounds, comprising Camptosar™ (irinotecan HCl), Hycamtin™ (topotecan HCl), and other compounds derived from camptothecin and analogs thereof; podophyllotoxin derivatives, such as etoposide, teniposide, and mitoxantrone; alkylating agents, such as cisplatin, cyclophosphamide, nitrogen mustard, trimethylenethiophosphoramide, carmustine, busulfan, chlorambucil, brequinar, uracil mustard, and dacarbazine; antimetabolites, such as cytarabine, 5-fluorouracil, methotrexate, mercaptopurine, azathioprine, and procarbazine; antibiotics, such as doxorubicin, bleomycin, dactinomycin, daunorubicin, mitomycin, mitomycin C, and streptozotocin; and other chemotherapeutic agents, comprising but not limited to antitumor antibodies, dacarbazine, azacitidine, amsacrine, melphalan, ifosfamide, and mitoxantrone. In some embodiments, the additional therapeutic agent is selected from one or more of epirubicin, oxaliplatin, and 5-fluorouracil. In some embodiments, the additional therapeutic agent comprises, but is not limited to, antiangiogenic agents, such as anti-VEGF antibodies (comprising humanized and chimeric antibodies, anti-VEGF aptamers, and antisense oligonucleotides) and other angiogenesis inhibitors, such as angiostatin, endostatin, interferons, interleukin-1 (comprising α and β), interleukin-12, retinoic acid, and tissue inhibitors of metalloproteinases-1 and -2.

In one embodiment, the anti-CLDN6 antibody, immunoconjugate, chimeric receptor-modified host cell, pharmaceutical composition, or kit of the present invention is administered in combination with an inhibitor of inhibitory molecules. The inhibitory molecules comprise PD1, PD-L1, CTLA-4, TIM3, LAG3, VISTA, BTLA, TIGIT, LAIR1, CD160, 2B4, and TGFRβ. Inhibitory nucleic acids, such as dsRNA, siRNA, or shRNA, can be used to suppress the expression of inhibitory molecules in CAR-expressing cells. In one embodiment, the inhibitor is a shRNA. In one embodiment, the inhibitory molecules in CAR-expressing cells are suppressed. In these embodiments, the dsRNA molecule inhibiting the expression of the inhibitory molecule can be linked to a nucleic acid encoding the components (e.g., all components) of the CAR. In one embodiment, an inhibitor of inhibitory signals can be, for example, an antibody or antibody fragment binding to the inhibitory molecule. For example, the active agent can be an antibody or antibody fragment binding to PD1, PD-L1, PD-L2, or CTLA4 (e.g., ipilimumab, tremelimumab).

In one embodiment, the anti-CLDN6 antibody, immunoconjugate, chimeric receptor-modified host cell, pharmaceutical composition, or kit of the present invention is administered in combination with an agent that ameliorates one or more related side effects. The side effects comprise but are not limited to CRS. Symptoms of CRS comprise high fever, nausea, transient hypotension, hypoxia, etc. The agent provided in the present invention for the administration in combination can manage elevated levels of soluble factors caused by the anti-CLDN6 antibody, immunoconjugate, chimeric receptor-modified host cell, pharmaceutical composition, or kit. Elevated soluble factors in individuals comprise IFN-γ, TNFα, IL-2, and/or IL-6. The agent administered to ameliorate side effects can be an active agent that neutralizes one or more of these soluble factors. Such active agents comprise but are not limited to steroids, TNFα inhibitors, and/or IL-6 inhibitors. TNFα inhibitors comprise but are not limited to Etanercept. IL-6 inhibitors comprise but are not limited to tocilizumab (toc).

In one embodiment, the anti-CLDN6 antibody, immunoconjugate, chimeric receptor-modified host cell, pharmaceutical composition, or kit of the present invention is administered in combination with cells expressing a chimeric antigen receptor targeting a molecule other than CLDN6. In one embodiment, it is administered in combination with an agent for treating diseases associated with CLDN6 expression. In one embodiment, the agent comprises an antibody, cell, RNA, vaccine, oncolytic virus, checkpoint inhibitor, BKT inhibitor, chemotherapeutic agent, radiotherapy agent, hormonal therapy agent, toxin, immunotherapeutic agent, or combinations thereof.

The anti-CLDN6 antibody, immunoconjugate, chimeric receptor-modified host cell, pharmaceutical composition, or kit of the present invention and another therapeutic agent can be administered simultaneously, in the same or separate compositions, or sequentially. In one embodiment, the anti-CLDN6 antibody, immunoconjugate, chimeric receptor-modified host cell, pharmaceutical composition, or kit of the present invention may be administered firstly, followed by the other therapeutic agent. In one embodiment, the other therapeutic agent may be administered firstly, followed by the anti-CLDN6 antibody, immunoconjugate, chimeric receptor-modified host cell, pharmaceutical composition, or kit of the present invention.

### Kits

Kits comprising the antibodies, immunoconjugates, chimeric receptors, nucleic acids, or host cells described herein are also provided in the present invention. In some embodiments, the kit may comprise a therapeutic or prophylactic composition containing an effective amount of one or more unit dosage forms of the antibodies, chimeric receptors, nucleic acids, or host cells described herein. In some embodiments, the kit contains a sterile container that may hold the therapeutic or prophylactic composition; such container may be a box, ampoule, bottle, vial, tube, bag, blister pack, or other suitable forms known in the art. Such container may be made of plastic, glass, laminated paper, metal foil, or other materials suitable for preserving pharmaceuticals. In some embodiments, the kit comprises the antibody, immunoconjugate, chimeric receptor, nucleic acid, or host cell described herein, along with an instruction for administering them to an individual. The instructions typically comprise methods for using the antibody, immunoconjugate, chimeric receptor, nucleic acid, or host cell described herein to treat or prevent a cancer or tumor. In some embodiments, the kit comprises the host cells described herein and may contain about 1×10⁴ to about 1×10⁶ cells. In some embodiments, the kit may comprise at least about 1×10⁵ cells, at least about 1×10⁶ cells, at least about 1×10⁷ cells, at least about 4×10⁷ cells, at least about 5×10⁷ cells, at least about 6×10⁷ cells, at least about 8×10⁷ cells, at least about 9×10⁷ cells, at least about 1×10⁸ cells, at least about 2×10⁸ cells, at least about 3×10⁸ cells, at least about 4×10⁸ cells, at least about 5×10⁸ cells, at least about 6×10⁸ cells, at least about 7×10⁸ cells, at least about 8×10⁸ cells, at least about 9×10⁸ cells, at least about 1×10⁹ cells, at least about 2×10⁹ cells, at least about 3×10⁹ cells, at least about 4×10⁹ cells, at least about 5×10⁹ cells, at least about 6×10⁹ cells, at least about 8×10⁹ cells, at least about 9×10⁹ cells, at least about 1×10¹⁰ cells, at least about 2×10¹⁰ cells, at least about 3×10¹⁰ cells, at least about 4×10¹⁰ cells, at least about 5×10¹⁰ cells, at least about 6×10¹⁰ cells, at least about 7×10¹⁰ cells, at least about 8×10¹⁰ cells, at least about 9×10¹⁰ cells, at least about 1×10¹¹ cells, at least about 2×10¹¹ cells, at least about 3×10¹¹ cells, at least about 4×10¹¹ cells, at least about 5×10¹¹ cells, at least about 8×10¹¹ cells, at least about 9×10¹¹ cells, or at least about 1×10¹² cells. For example, the kit may comprise about 5×10¹⁰ cells. In another example, the kit may comprise 3×10⁶ cells; and the cells can be expanded to about 5×10¹⁰ cells and administered to a subject.

In some embodiments, the kit may comprise allogeneic cells. In some embodiments, the kit may comprise cells that may comprise genomic modifications. In some embodiments, the kit may contain “ready-to-use” cells. In some embodiments, the kit may comprise cells that can be expanded for clinical use. In certain cases, the kit may contain materials for research purposes.

In some embodiments, the instruction comprises at least one of the following: a description of the therapeutic agent; dosage regimens and administration for treating or preventing a tumor or symptoms thereof; precautions, warnings, contraindications, overdose information, adverse reactions, animal pharmacology, clinical studies, and/or references. The instruction may be printed directly on the container (if present), provided as a label on the container, or as a separate sheet, booklet, card, or folder inside or with the container. In some embodiments, the instruction provides methods for administering the antibody of the present invention to treat or prevent a tumor. In certain cases, the instructions provide methods for administering the antibody of the present invention before, after, or concurrently with chemotherapeutic agents.

### Methods for Diagnosis/Detection/Treatment

The term “modulation” or “regulate” refers to a positive or negative change. Examples of the modulation comprise changes of 1%, 2%, 10%, 25%, 50%, 75%, or 100%. In a specific embodiment, it refers to a negative change.

The term “treatment” refers to an intervention aimed at altering the course of a disease, which may be preventive or be clinically conducted during a pathological process. Therapeutic effects comprise but are not limited to preventing the occurrence or recurrence of a disease, alleviating symptoms, reducing any direct or indirect pathological consequences of the disease, preventing metastasis, slowing disease progression, improving or mitigating the condition, or ameliorating the prognosis. The term does not imply a complete cure for the disease, the complete elimination of any symptoms, or effectiveness against all symptoms or outcomes.

The term “anti-tumor effect” refers to a biological effect which may manifest in various forms, comprising but not limited to, for example, reduction in the tumor volume, decrease in the number of tumor cells, reduction in metastatic tumors, increased lifespan, reduced tumor cell proliferation, reduced tumor cell survival, or improvement in various physiological symptoms related to a cancer. “Anti-tumor effects” may also manifest as the ability of the peptides, polynucleotides, cells, and antibodies of the present invention to prevent tumor formation from the beginning.

The term “prevention” refers to providing preventive methods for the occurrence or recurrence of a disease in individuals predisposed to developing the disease but not yet diagnosed, or interventions aimed at preventing the disease (e.g., rejections caused by cell transplantation) before it occurs, or prophylactic treatment of the disease or disease state.

The term “autologous” refers to any substance obtained from an individual and subsequently reintroduced into the same individual.

The term “allogeneic” refers to any substance introduced into an individual that originates from a different individual of the same species. When two or more individuals differ genetically at one or more loci, they are considered as allogeneic. In some aspects, the allogeneic material from individuals of the same species may be genetically different enough to elicit antigenic interactions.

The term “xenogeneic” refers to a graft derived from an individual of a different species.

The term “tumor” or “cancer” refers to a disease characterized by rapid and uncontrolled abnormal cell growth. Tumor or cancer cells may spread locally or to other parts of the body via the bloodstream and lymphatic system.

The term “disease associated with CLDN6 expression” or “disease related to CLDN6 expression” comprises but is not limited to a disease associated with CLDN6 expression or conditions related to cells expressing CLDN6, such as ovarian cancer, breast cancer, cervical cancer, gastric cancer, lung cancer, testicular cancer, germ cell and embryonic tumors, ovarian epithelial cancer, non-small cell lung cancer, non-squamous non-small cell lung cancer, endometrial cancer, etc.

The term “detection” comprises a quantitative or qualitative detection. The antibody of the present invention can be used to detect the presence of CLDN6 in biological samples, comprising blood, serum, cells, or tissues.

The term “tumor antigen” refers to an antigen that emerges or is overexpressed during the development and progression of hyperproliferative diseases. In certain aspects, the hyperproliferative disease of the present invention refers to a tumor.

The tumor antigen of the present invention may be a solid tumor antigen or hematologic tumor antigen.

The tumor antigen of the present invention comprises but not limited to: thyroid-stimulating hormone receptor (TSHR); CD171; CS-1; C-type lectin-like molecule-1; ganglioside GD3; Tn antigen; CD19; CD20; CD22; CD30; CD70; CD123; CD138; CD33; CD44; CD44v7/8; CD38; CD44v6; B7H3 (CD276), B7H6; KIT (CD117); interleukin-13 receptor subunit α (IL-13Rα); interleukin-11 receptor α (IL-11Rα); prostate stem cell antigen (PSCA); prostate-specific membrane antigen (PSMA); carcinoembryonic antigen (CEA); NY-ESO-1; HIV-1 Gag; MART-1; gp100; tyrosinase; mesothelin; EpCAM; protease serine 21 (PRSS21); vascular endothelial growth factor receptor, vascular endothelial growth factor receptor 2 (VEGFR2); Lewis (Y) antigen; CD24; platelet-derived growth factor receptor β (PDGFR-β); stage-specific embryonic antigen-4 (SSEA-4); cell surface-associated mucin 1 (MUC1), MUC6; epidermal growth factor receptor family and its mutants (EGFR, EGFR2, ERBB3, ERBB4, EGFRvIII); neural cell adhesion molecule (NCAM); carbonic anhydrase IX (CAIX); LMP2; ephrin type-A receptor 2 (EphA2); fucosyl-GM1; sialyl Lewis adhesion molecule (sLe); ganglioside GM3; TGS5; high-molecular-weight melanoma-associated antigen (HMWMAA); O-acetyl-GD2 ganglioside (OAcGD2); folate receptor; tumor vascular endothelial marker 1 (TEM1/CD248); tumor vascular endothelial marker 7-related (TEM7R); Claudin6, Claudin18.2, Claudin18.1; ASGPR1; CDH16; 5T4; 8H9; αvβ6 integrin; B-cell maturation antigen (BCMA); CA9; κ light chain (kappa light chain); CSPG4; EGP2, EGP40; FAP; FAR; FBP; embryonic acetylcholine receptor; HLA-A1, HLA-A2; MAGEA1, MAGE3; KDR; MCSP; NKG2D ligands; PSC1; ROR1; Sp17; SURVIVIN; TAG72; TEM1; fibronectin; tenascin; carcinoembryonic variant of tumor necrosis zone; G protein-coupled receptor class C group 5 member D (GPRC5D); X chromosome open reading frame 61 (CXORF61); CD97; CD179a; anaplastic lymphoma kinase (ALK); polysialic acid; placenta-specific 1 (PLAC1); hexose portion of globoH glycoceramide (GloboH); breast differentiation antigen (NY-BR-1); uroplakin 2 (UPK2); hepatitis A virus cellular receptor 1 (HAVCR1); adrenaline antigen (NY-BR-1); uroplakin 2 (UPK2); hepatitis A virus cellular receptor 1 (HAVCR1); adrenergic receptor β3 (ADRB3); pannexin 3 (PANX₃); G protein-coupled receptor 20 (GPR20); lymphocyte antigen 6 complex locus K9 (LY6K); olfactory receptor 51E2 (OR51E2); TCRγ alternate reading frame protein (TARP); Wilms tumor protein (WT1); ETS translocation variant gene 6 (ETV6-AML); sperm protein 17 (SPA17); X antigen family member 1A (XAGE1); angiopoietin-binding cell surface receptor 2 (Tie2); melanoma cancer testis antigen-1 (MAD-CT-1); melanoma cancer testis antigen-2 (MAD-CT-2); Fos-related antigen 1; p53 mutant; human telomerase reverse transcriptase (hTERT); sarcoma translocation breakpoint; melanoma inhibitor of apoptosis (ML-IAP); ERG (transmembrane protease serine 2 (TMPRSS2) ETS fusion gene); N-acetylglucosaminyltransferase V (NA17); paired box protein Pax-3 (PAX3); androgen receptor; cyclin B1; v-myc avian myelocytomatosis viral oncogene neuroblastoma-derived homolog (MYCN); Ras homolog family member C (RhoC); cytochrome P450 1B1 (CYP1B1); CCCTC-binding factor (zinc finger protein)-like (BORIS); squamous cell carcinoma antigen recognized by T cells 3 (SART3); paired box protein Pax-5 (PAX5); proacrosin-binding protein sp32 (OY-TES1); lymphocyte-specific protein tyrosine kinase (LCK); A-kinase anchor protein 4 (AKAP-4); synovial sarcoma X breakpoint 2 (SSX2); CD79a; CD79b; CD72; leukocyte-associated immunoglobulin-like receptor 1 (LAIR1); Fc fragment of IgA receptor (FCAR); leukocyte immunoglobulin-like receptor subfamily member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); glypican-3 (GPC3); Fc receptor-like 5 (FCRL5); immunoglobulin lambda-like polypeptide 1 (IGLL1). Preferably, the tumor antigen is CSI, Claudin18.2, GPC3, BCMA, or CD19.

Pathogen antigens comprise: viral, bacterial, fungal, protozoan, or parasitic antigens; and viral antigens comprise: cytomegalovirus antigens, Epstein-Barr virus antigens, human immunodeficiency virus antigens, or influenza virus antigens.

The term “individual” or “subject” is intended to comprise a living organism capable of eliciting an immune response and refers to any animal, such as a mammal or marsupial. Individuals of the present invention comprise but are not limited to humans, non-human primates (e.g., rhesus monkeys or other types of macaques), mice, pigs, horses, donkeys, cattle, sheep, rats, and any species of poultry.

The term “effective amount” refers to an amount that provides therapeutic or prophylactic benefits, thereby achieving the desired therapeutic or preventive outcomes when administered at a certain dosage for a necessary duration. The amount can be determined by a physician based on individual differences, such as age, weight, tumor size, extent of infection, degree of metastasis, etc.

Any of the anti-CLDN6 antibody, immunoconjugate, chimeric receptor-modified host cell, pharmaceutical composition, or kit provided herein can be used in therapeutic methods.

By eliciting an antigen-specific response against CLDN6, any anti-CLDN6 antibody, immunoconjugate, chimeric receptor-modified host cell, pharmaceutical composition, or kit provided in the present invention provides one or more of the following: targeting and destruction of CLDN6-expressing tumor cells, reduction or elimination of a tumor, promotion of immune cell infiltration into tumor sites, and enhancement/prolongation of anti-tumor responses. Since CLDN6 is expressed at undetectable levels in normal (i.e., non-cancerous) tissues, it is believed that the anti-CLDN6 antibody, immunoconjugate, chimeric receptor-modified host cell, pharmaceutical composition, or kit provided in the present invention will not target/destroy normal tissues and cells.

In one aspect, any anti-CLDN6 antibody, immunoconjugate, chimeric receptor-modified host cell, pharmaceutical composition, or kit is provided for use as a medicament. In another aspect, any anti-CLDN6 antibody, immunoconjugate, chimeric receptor-modified host cell, pharmaceutical composition, or kit is provided for treating a disease. In certain embodiments, any anti-CLDN6 antibody, immunoconjugate, chimeric receptor-modified host cell, pharmaceutical composition, or kit is provided for use in therapeutic methods. In certain embodiments, any anti-CLDN6 antibody, immunoconjugate, chimeric receptor-modified host cell, pharmaceutical composition, or kit is provided in the present invention for use in a method of treating an individual with a disease, the method comprising administering to the individual an effective amount of any anti-CLDN6 antibody, immunoconjugate, chimeric receptor-modified host cell, pharmaceutical composition, or kit. In one embodiment, the method further comprises administering to the individual an effective amount of at least one additional therapeutic agent. Preferably, the “individual” is a human.

In another aspect, the use of any anti-CLDN6 antibody, immunoconjugate, chimeric receptor-modified host cell, pharmaceutical composition, or kit is provided in the present invention for preparing or formulating a medicament. In one embodiment, the medicament is used for treating a disease. In another embodiment, the medicament is used in a method of treating a disease, the method comprising administering to an individual with the disease an effective amount of the medicament. In one embodiment, the method further comprises administering to the individual an effective amount of at least one additional therapeutic agent. Preferably, the “individual” is a human.

In another aspect, a method for treating a disease is provided in the present invention. In one embodiment, the method comprises administering to an individual with a disease associated with CLDN6 expression an effective amount of any anti-CLDN6 antibody, immunoconjugate, chimeric receptor-modified host cell, pharmaceutical composition, or kit. In one embodiment, the method further comprises administering to the individual an effective amount of at least one additional therapeutic agent. Preferably, the “individual” is a human.

In another aspect, a pharmaceutical formulation comprising any anti-CLDN6 antibody, immunoconjugate, chimeric receptor-modified host cell, pharmaceutical composition, or kit provided herein for use in any of the therapeutic methods as said above is provided in the present invention. In one embodiment, the pharmaceutical formulation comprises any anti-CLDN6 antibody, immunoconjugate, chimeric receptor-modified host cell, pharmaceutical composition, or kit provided herein and a pharmaceutically acceptable carrier. In another embodiment, the pharmaceutical formulation comprises any anti-CLDN6 antibody, immunoconjugate, chimeric receptor-modified host cell, pharmaceutical composition, or kit provided herein and at least one additional therapeutic agent.

In another aspect, the pharmaceutical formulation is used for treating a disease. In one embodiment, the pharmaceutical formulation is administered to an individual with the disease. Preferably, the “individual” according to any of the above embodiments is a human.

In another aspect, a method for preparing a medicament or pharmaceutical formulation is provided in the present invention, comprising mixing any anti-CLDN6 antibody, immunoconjugate, chimeric receptor-modified host cell, pharmaceutical composition, or kit provided herein with a pharmaceutically acceptable carrier, for example, for use in any of the therapeutic methods as said above. In one embodiment, the method for preparing a medicament or pharmaceutical formulation further comprises adding at least one additional therapeutic agent to the medicament or pharmaceutical formulation.

Any anti-CLDN6 antibody, immunoconjugate, chimeric receptor-modified host cell, pharmaceutical composition, or kit of the present invention can be used alone or in combination with other agents for the treatment. Alternatively, any anti-CLDN6 antibody, immunoconjugate, chimeric receptor-modified host cell, pharmaceutical composition, or kit of the present invention can be co-administered with at least one additional therapeutic agent.

Such combination therapies as described above comprise the combined administration (wherein two or more therapeutic agents are contained in the same or separate formulations) and separate administration, where the administration of any anti-CLDN6 antibody, immunoconjugate, chimeric receptor-modified host cell, pharmaceutical composition, or kit of the present invention may occur before, concurrently with, and/or after the administration of the additional therapeutic agent or reagent. In one embodiment, the administration of any anti-CLDN6 antibody, immunoconjugate, chimeric receptor-modified host cell, pharmaceutical composition, or kit of the present invention and the administration of the additional therapeutic agent occur within about one month of each other, or within about one, two, or three weeks, or within about one, two, three, four, five, or six days of each other.

Any anti-CLDN6 antibody, immunoconjugate, chimeric receptor-modified host cell, pharmaceutical composition, or kit of the present invention (and any additional therapeutic agents) can be administered by any suitable means, comprising parenteral, pulmonary, or intranasal administration, and, if therapeutically required, intralesional administration. The parenteral infusion comprises intramuscular, intravenous, intraarterial, intraperitoneal, or subcutaneous administration. The administration can be conducted via any suitable route, for example, by injection, such as intravenous or subcutaneous injection, depending on whether the administration is brief or long-term. Various administration regimens are contemplated herein, comprising but not limited to a single administration or multiple administrations at multiple time points, bolus administration, and pulse infusion.

A formulation administered to an individual comprising a population of immunoreactive cells comprises an effective number of immunoreactive cells for treating and/or preventing a specific indication or disease. Therefore, a therapeutically effective population of immunoreactive cells can be administered to an individual. Typically, a formulation comprising about 1×10⁴ to about 1×10¹⁰ immunoreactive cells is administered. In most cases, the formulation will comprise about 1×10⁵ to about 1×10⁹ immunoreactive cells, about 5×10⁵ to about 5×10⁸ immunoreactive cells, or about 1×10⁶ to about 1×10⁷ immunoreactive cells. However, the number of CAR immunoreactive cells administered to an individual will vary widely depending on factors, such as the location, origin, identity, extent, and severity of the tumor, the age and physical condition of the individual to be treated, etc. A physician will ultimately determine the appropriate dosage to be used.

In some embodiments, chimeric receptors are used to stimulate host cell-mediated immune responses. For example, a T cell-mediated immune response is an immune response involving T cell activation. Activated antigen-specific cytotoxic T cells can induce apoptosis in target cells displaying exogenous antigen epitopes on their surface, such as tumor cells displaying tumor antigens. In other embodiments, chimeric antigen receptors are used to provide anti-tumor immunity in mammals. Due to the T cell-mediated immune response, a subject will develop anti-tumor immunity.

In certain cases, a method for treating a subject with a tumor may involve administering to the subject in need of the treatment one or more host cells of the present invention. The host cells can bind to tumor target molecules and induce tumor cell death. As described above, methods for treating pathogen infections in individuals is also provided in the present invention, comprising administering to the individual a therapeutically effective amount of the host cells of the present invention.

The administration frequency of the immunoreactive cells of the present invention will depend on factors comprising the disease being treated, the specific elements of the immunoreactive cells, and the mode of administration. For example, the administration may be conducted four times, three times, twice, or once daily, every other day, every three days, every four days, every five days, every six days, once a week, every eight days, every nine days, every ten days, once a week, or twice a month. As described herein, since the immunoreactive cells of the present application exhibit improved viability, they can be administered not only at lower therapeutically effective amounts compared with similar immunoreactive cells that do not express exogenous type I interferon but also at lower frequencies to achieve at least similar and preferably more significant therapeutic effects.

### Advantages of the Invention

Humanized antibodies that specifically recognize CLDN6 is provided in the present invention. CAR-T cells prepared from such antibodies exhibit good killing effects on target cells in vitro and in vivo.

It should be appreciated that, for clarity, certain features of the invention described in the context of individual embodiments may also be provided in combination in a single embodiment. On the contrary, for brevity, various features of the invention described in the context of a single embodiment may also be provided separately or in any suitable subcombination. All combinations of the embodiments of the invention are expressly comprised in and disclosed herein, as if each and every combination were individually and explicitly disclosed. Furthermore, all subcombinations of the various embodiments and elements therein are also expressly comprised in and disclosed herein, as if each and every such subcombination were individually and clearly disclosed herein.

### Example 1. Preparation of Humanized CLDN6 Antibodies

In this example, the murine antibody SC27.105 (from WO2016073649A1) was used as the parent antibody. The sequence of the parent antibody was compared with germline sequences from the IMGT database to screen for CDR grafting template 1 for the heavy chain and CDR grafting template 2 for the light chain of SC27.105. The LCDR regions of the SC27.105 antibody were used to replace the CDR regions of Template 2, thereby constructing the light chain variable region VL of the humanized antibody H1 (amino acid sequence shown in SEQ ID NO: 3). The serine(S) at position 5 in the heavy chain CDR2 of the SC27.105 antibody was mutated to alanine (A). Afterwards, the HCDR1, mutated HCDR2, and HCDR3 regions of the parent antibody were used to replace the CDR regions of Template 2, thereby constructing the heavy chain variable region VH of the humanized antibody H1 (amino acid sequence shown in SEQ ID NO: 1). The framework regions of the VH amino acid sequence of humanized antibody H1 correspond to positions 1-30, 36-49, 67-98 and 113-123 in SEQ ID NO: 1. The framework regions of the VL amino acid sequence of humanized antibody H1 correspond to positions 1-23, 35-49, 57-88 and 97-106 in SEQ ID NO: 3.

Heavy chain variable region of H1, CDR regions are underlined,
and the framework regions comprise remaining sequences except CDRs.
(SEQ ID NO: 1)
EVQLVQSGAEVKKPGASVKVSCKASGYTFTGYYMNWVRQAPGQGLEWMGEINPATGST
TYNQKFKARVTITRDTSTSTAYMELSSLRSEDTAVYYCARRDYYYGSGFYAMDYWGQGT
LVTVSS
Light chain variable region of H1, CDR regions are underlined,
and the framework regions comprise remaining sequences except CDRs.
(SEQ ID NO: 3)
DIQMTQSPSSLSASVGDRVTITCQASQSVSNNLNWYQQKPGKAPKLLIYGASKLEDGVPSR
FSGSGSGTDFTFTISSLQPEDFATYYCLQHRYLWTFGQGTKVEIK

### Example 2. Specificity and Affinity Detection of Humanized Antibody H1

### 1. Construction of Cell Lines

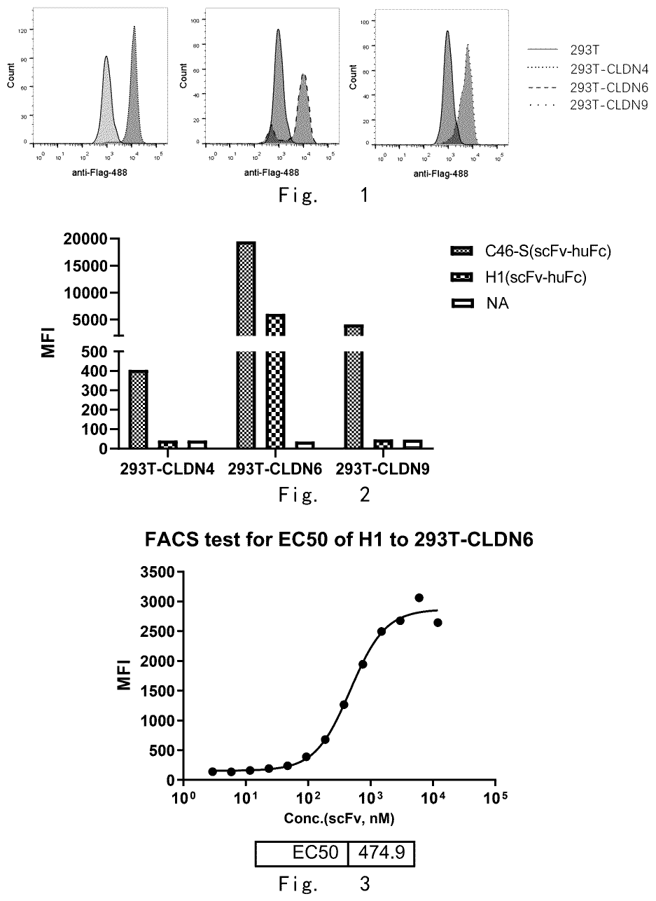

Human CLDN6 (SEQ ID NO: 32), CLDN4 (SEQ ID NO: 31) and CLDN9 (SEQ ID NO: 33) were transfected into 293T cells by adopting conventional molecular biology techniques via lentivirus, respectively. Positive clones were selected by limiting dilution to construct stable cell lines 293T-CLDN6, 293T-CLDN4, and 293T-CLDN9 (FIG. 1).

### 2. Detection of Binding EC50 of Antibody H1 to Cell Lines

The above 293T-CLDN6, 293T-CLDN4, and 293T-CLDN9 cells (2×10⁵ cells/well) were added to a 96-well round-bottom plate, washed with PBS, and incubated with antibody H1 at 4° C. After centrifugation, the supernatant was discarded, and the cells were washed with PBS and incubated with Goat-anti-Mouse FITC at 4° C. After centrifugation, the supernatant was discarded, and the cells were washed and detected by flow cytometry. FlowJo v.X.0.7 was used for statistics and GraphPad Prism 8.0 was used for plotting.

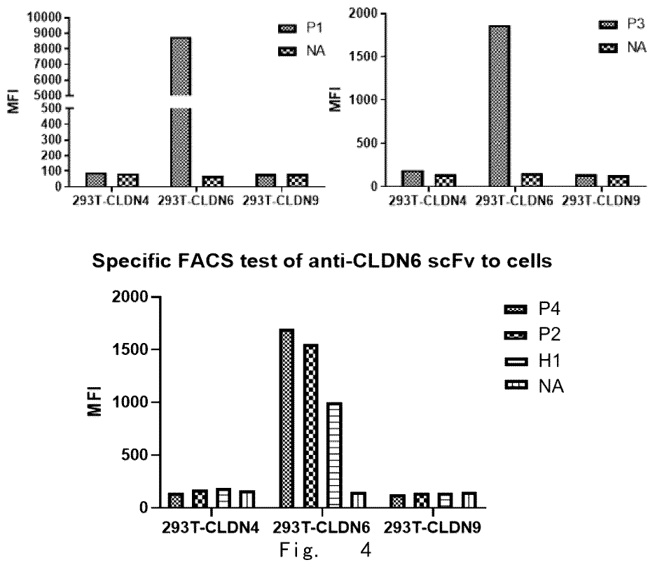

Results showed that antibody H1 (scFv-huFc, 10 μg/mL) specifically bound to 293T-CLDN6 cells with an EC50 of 474.9 nM (FIGS. 2, 3), but did not bind to 293T-CLDN4 or 293T-CLDN9 cells (FIG. 2). The control antibody C46-S(scFv-huFc, 10 μg/mL, from WO2015150327A1) showed significant binding to 293T-CLDN4, 293T-CLDN6, and 293T-CLDN9 cells.

### Example 3. Screening and Characterization of H1 Antibody Mutants

To improve the affinity of antibody H1, conventional molecular biology techniques were used to randomize mutations in the light chain CDR3 and heavy chain CDR3 of antibody H1, thereby constructing phage libraries H1-VH and H1-VL, each with a library size of 1×10⁹. The phage libraries were first co-incubated with 293T cells for 1-2 h, and centrifuged, and the supernatant was co-incubated with 293T-CLDN6 cells for 1-2 h. After being washed, the phages bound to the cells were eluted, neutralize the eluent, and then infect E. coli TG1. After expansion, the phages were purified for the next round of screening. After repeating screened as said above for 2-3 rounds, about 1,000 monoclone were selected for ELISA detection, thereby obtaining over 70 clones with strong binding to 293T-CLDN6 cells. After being sequenced, 23 sequences were obtained. The 23 clones were subjected to prokaryotic expression and purified to obtain single chain antibodies (scFv), and flow cytometry showed that 4 clones (named P1, P2, P3, P4 (antibody concentration was 10 μg/mL)) specifically bound to 293T-CLDN6 cells (FIG. 4, wherein the primary antibody in the blank group (NA) was PBS).

Antibody P1: amino acid sequence of VH shown in SEQ ID NO: 5, amino acid sequence of VL shown in SEQ ID NO: 3; amino acid sequence of HCDR3 shown in SEQ ID NO: 41; amino acid sequence of scFv shown in SEQ ID NO: 14.

Antibody P2: amino acid sequence of VH shown in SEQ ID NO: 1, amino acid sequence of VL shown in SEQ ID NO: 7; amino acid sequence of LCDR3 shown in SEQ ID NO: 42; amino acid sequence of scFv shown in SEQ ID NO:15.

Antibody P3: amino acid sequence of VH shown in SEQ ID NO: 1, amino acid sequence of VL shown in SEQ ID NO: 9; amino acid sequence of LCDR3 shown in SEQ ID NO: 43; amino acid sequence of scFv shown in SEQ ID NO: 16.

Antibody P4: amino acid sequence of VH shown in SEQ ID NO: 1, amino acid sequence of VL shown in SEQ ID NO: 11; amino acid sequence of LCDR3 shown in SEQ ID NO: 44; amino acid sequence of scFv shown in SEQ ID NO: 17.

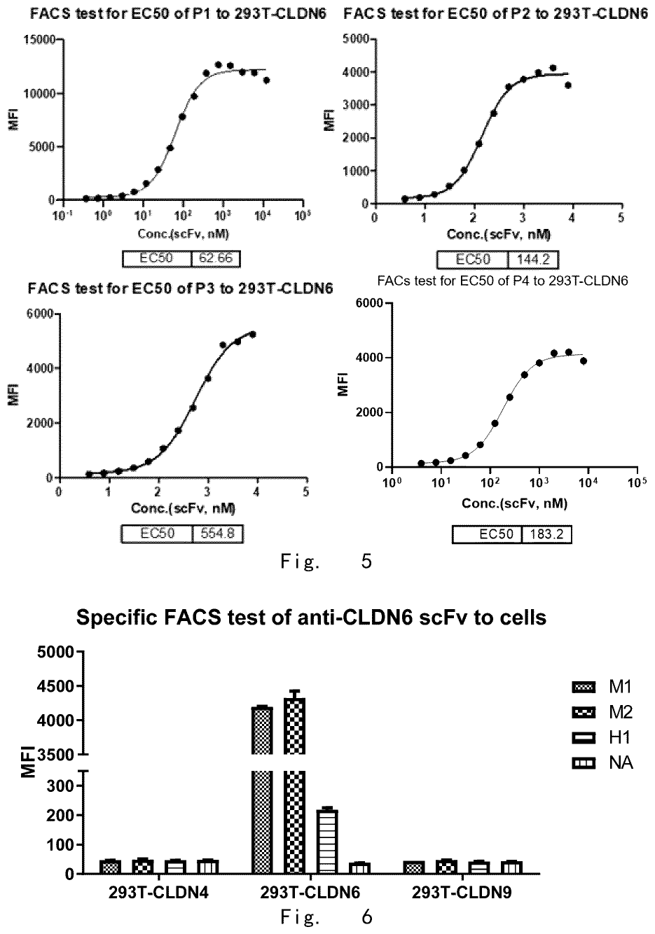

Flow cytometry results (FIG. 5) showed that antibodies P1, P2, P3 and P4 exhibited strong binding affinity to 293T-CLDN6 cells, with EC50 values of 62.66 nM, 144.2 nM, 554.8 nM, and 183.2 nM, respectively.

### Example 4. Specificity and Affinity Detection of H1 Mutants

The heavy chain variable region of antibody P1 from Example 3 was combined with the light chain variable regions of antibodies P2 and P3 by using molecular cloning, respectively, so as to obtain antibodies M1 and M2. After being prokaryotically expressed and puried, purified single chain antibodies (scFv) were obtained. M1 and M2 were co-incubated with 293T-CLDN6, 293T-CLDN4 and 293T-CLDN9 cells, respectively, and bound scFv were fluorescently labeled for the detection by flow cytometry. Data were analyzed using FlowJo to calculate mean fluorescence intensity (MFI).

Results (FIG. 6) showed that M1 and M2 (scFv, 5 μg/mL) specifically bound to 293T-CLDN6 cells but not to 293T-CLDN4 or 293T-CLDN9 cells.

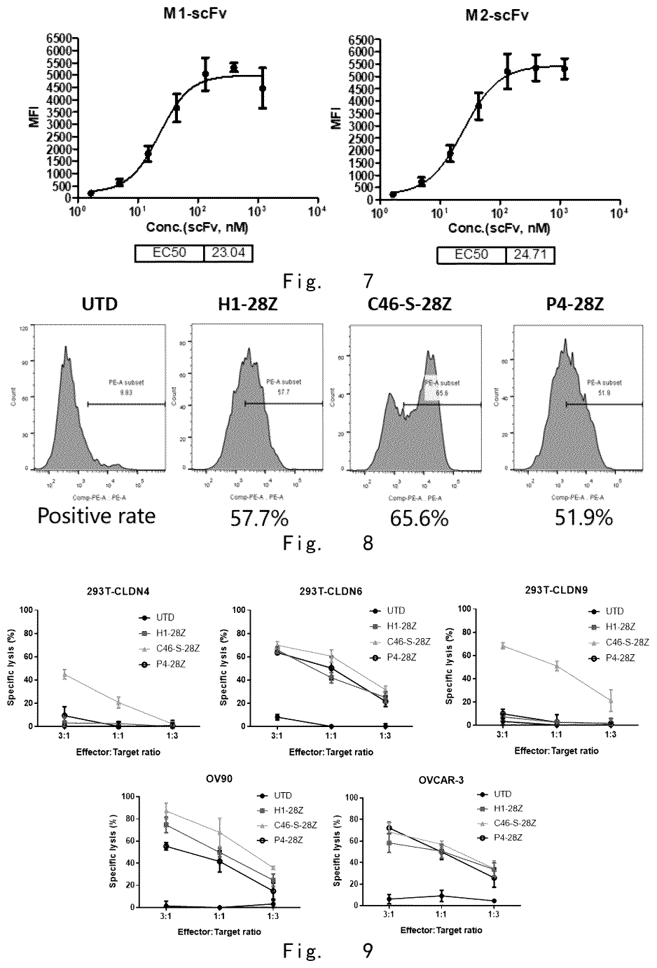

FIG. 7 showed that M1 and M2 exhibited significant binding to 293T-CLDN6 cells, with EC50 values of 23.04 nM and 24.71 nM, respectively, which represents a nearly 20-fold improvement in affinity compared with antibody H1.

### Example 5. Preparation of CLDN6 CAR-T Cells

### 1. Construction of CAR Vector

Lentiviral plasmids PRRLSIN-cPPT.EF-1α-H1-28Z and PRRLSIN-cPPT.EF-1α-P4-28Z expressing the second-generation chimeric antigen receptors (CARs) comprise antibodies H1 and P4 were constructed by using PRRLSIN-cPPT.EF-1α as the vector. The amino acid sequence of the obtained H1-28Z is shown in SEQ ID NO: 48 and the amino acid sequence of the obtained P4-28Z is shown in SEQ ID NO: 49, respectively.

The amino acid sequence of the control group C46-S-28Z is shown in SEQ ID NO: 51.

### 2. Preparation of CAR-T Cells

CAR-T cells were prepared using conventional methods. PBMC cells were activated by magnetic beads with anti-CD3 and CD28 antibodies, and then cultured to obtain T cells. CAR-T cells were obtained from T cells infected with lentivirus.

Lentivirus was packaged using the calcium phosphate method, the virus supernatant was purified with PEG8000/NaCl, and the purified viruses were used to infect T cells (MOI=20) which were activated with CD3/CD28 magnetic beads for 48 hours, so as to obtain CAR-T cells expressing H1-28Z, P4-28Z, or C46-S-28Z, with untransduced T cells (UTD) as the control. On the 5th Day after infection, CAR positivity rate was assessed by FACS using Biotin-anti-F(ab′) 2-488 (Jackson ImmunoResearch) as the primary antibody and SA-PE (eBioscience) as the second antibody. Results are shown in FIG. 8.

### Example 6. In Vitro Specific Killing of CLDN6 CAR-T Cells on Target Cells

Target cells, 293T-CLDN4 cell, 293T-CLDN6 cell, 293T-CLDN9 cell, OVCAR3 cell (human ovarian cancer cells, ATCC), and OV90 cell (human ovarian cancer cells, ATCC) were adjusted to 0.2×10⁶/mL using 1640 medium, respectively, and 50 μL (i.e., 10,000 cells/well) was added to a 96-well plate. 50 μL of Effector cells (CAR-T cells, UTD controls) were added at an effector-to-target ratio of 1:1 and co-cultured for 16 h at 37° C. The supernatant was analyzed using an LDH assay kit, and measured on a microplate reader at OD490.

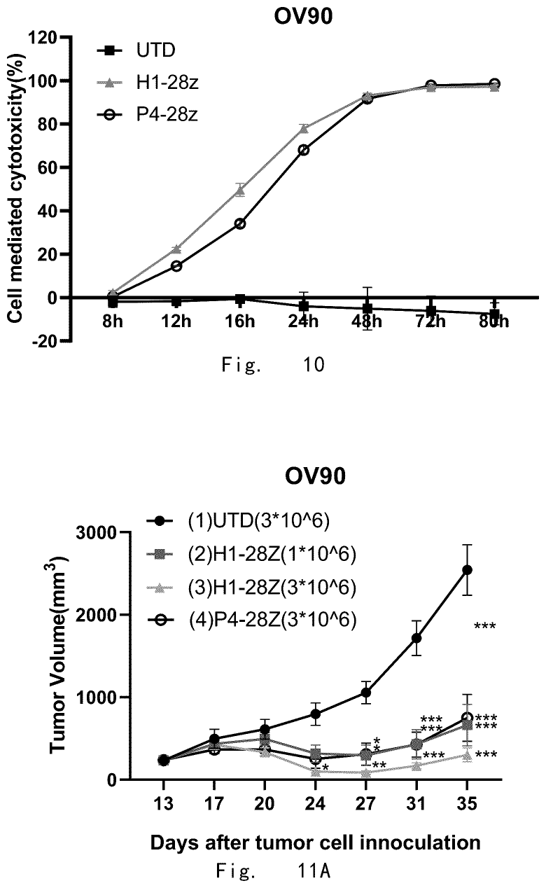

Results are shown in FIG. 9. H1 CAR-T and P4 CAR-T cells did not exhibit killing effects on 293T-CLDN4 or 293T-CLDN9 cells but exhibited significant killing effects on CLDN6-expressing 293T-CLDN6, OVCAR3, and OV90 cells with 60%-90% killing rate at E:T 3:1. The results showed that H1 CAR-T and P4 CAR-T cells have specific in vitro killing effects on CLDN6-expressing cells. C46-S CAR-T cells showed non-specific killing effects.

### Example 7. RTCA Detection and Analysis of In Vitro Killing Effects of CLDN6 CAR-T on Ovarian Cancer Cells

OV90 ovarian cancer cells were seeded in RTCA plates at 1E±04 cells/pore. After 20 h, UTD, H1-28Z-CAR T, or P4-28Z-CAR T cells were added at E:T 1:1. After being cultured for 8 h, 12 h, 16 h, 24 h, 48 h, 72 h and 80 h, target cell lysis was measured. As shown in FIG. 10, H1-28Z-CAR T and P4-28Z-CAR T cells showed significant killing effects on OV90 cancer cells, and the killing rate achieved >90% at 48 h, nearly 100% at 72 h.

### Example 8. Anti-Tumor Efficacy of CLDN6 CAR-T Cells on Subcutaneous Xenografts Tumor in NPG Mice Bearing Human Ovarian Cancer Cells

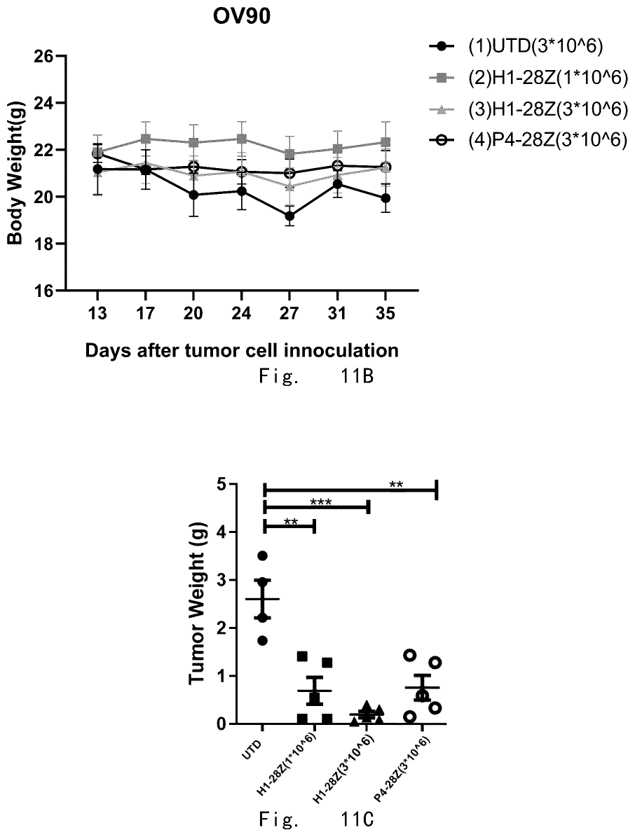

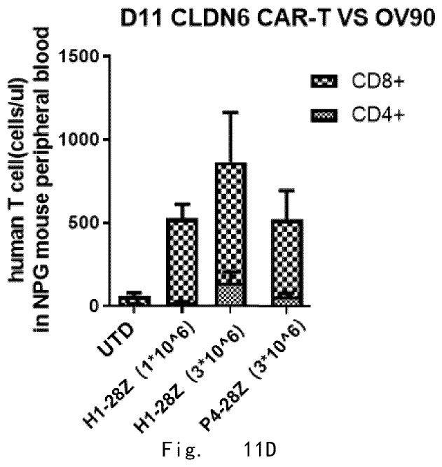

NPG mice were subcutaneously inoculated with 5×10⁶ OV90 cells. 13 days after inoculation, the average tumor volume achieved about 234 mm³. Mice were divided into 4 groups and intravenously injected with UTD (3×10⁶), H1-28Z (1×10⁶), H1-28Z (3×10⁶), or P4-28Z (3×10⁶) CAR-T cells via tail vein, respectively. Results are shown in FIG. 11A. On Day 22 after CAR-T injection, tumor inhibition rates were 74.11% in H1-28Z (1×10⁶) group, 88.26% in H1-28Z (3×10⁶) group, and 70.81% in P4-28Z group, compared with UTD. The body weights of the mice remained stable as shown in FIG. 11B. According to changes in the tumor weight, tumor inhibition rates were 73.46% in H1-28Z (1×10⁶) group, 92.41% in H1-28Z (3×10⁶) group, and 71.39% in P4-28Z group, as shown in FIG. 11C. On Day 11 after CAR-T injection, the persistence of human T cells in peripheral blood of mice in each group was shown in FIG. 11D.

Sequence listing

| | |
|---|---|
| 1 | EVQLVQSGAEVKKPGASVKVSCKASGYTFTGYYMNWVRQAPGQGLEW |
| | MGEINPATGSTTYNQKFKARVTITRDTSTSTAYMELSSLRSEDTAVYYCA |
| | RRDYYYGSGFYAMDYWGQGTLVTVSS |
| 2 | GAGGTGCAGCTGGTGCAGAGCGGCGCCGAGGTGAAGAAGCCCGGCGC |
| | CAGCGTGAAGGTGAGCTGCAAGGCCAGCGGCTACACCTTCACCGGCTA |
| | CTACATGAACTGGGTGCGGCAGGCCCCCGGCCAGGGCCTGGAGTGGAT |
| | GGGCGAGATCAACCCCGCCACCGGCAGCACCACCTACAACCAGAAGTT |
| | CAAGGCCCGGGTGACCATCACCCGGGACACCAGCACCAGCACCGCCTA |
| | CATGGAGCTGAGCAGCCTGCGGAGCGAGGACACCGCCGTGTACTACTG |
| | CGCCCGGCGGGACTACTACTACGGCAGCGGCTTCTACGCTATGGACTA |
| | CTGGGGCCAGGGCACCCTGGTGACCGTGAGCAGC |
| 3 | DIQMTQSPSSLSASVGDRVTITCQASQSVSNNLNWYQQKPGKAPKLLIYG |
| | ASKLEDGVPSRFSGSGSGTDFTFTISSLQPEDFATYYCLQHRYLWTFGQGT |
| | KVEIK |
| 4 | GACATCCAGATGACCCAGAGCCCCAGCAGCCTGAGCGCCAGCGTGGGC |
| | GACCGGGTGACCATCACCTGCCAGGCCAGCCAGAGCGTGAGCAACAA |
| | CCTGAACTGGTACCAGCAGAAGCCCGGCAAGGCCCCCAAGCTGCTGAT |
| | CTACGGCGCCAGCAAGCTGGAGGACGGCGTGCCCAGCCGGTTCAGCGG |
| | CAGCGGCAGCGGCACCGACTTCACCTTCACCATCAGCAGCCTGCAGCC |
| | CGAGGACTTCGCCACCTACTACTGCCTGCAGCACCGGTACCTGTGGAC |
| | CTTCGGCCAGGGCACCAAGGTGGAGATCAAG |
| 5 | EVQLVQSGAEVKKPGASVKVSCKASGYTFTGYYMNWVRQAPGQGLEW |
| | MGEINPATGSTTYNQKFKARVTITRDTSTSTAYMELSSLRSEDTAVYYCA |
| | RRDYYLGSNSYALDYWGQGTLVTVSS |
| 6 | GAGGTGCAGCTGGTGCAGAGCGGCGCCGAGGTGAAGAAGCCCGGCGC |
| | CAGCGTGAAGGTGAGCTGCAAGGCCAGCGGCTACACCTTCACCGGCTA |
| | CTACATGAACTGGGTGCGGCAGGCCCCCGGCCAGGGCCTGGAGTGGAT |
| | GGGCGAGATCAACCCCGCCACCGGCAGCACCACCTACAACCAGAAGTT |
| | CAAGGCCCGGGTGACCATCACCCGGGACACCAGCACCAGCACCGCCTA |
| | CATGGAGCTGAGCAGCCTGCGGAGCGAGGACACCGCCGTGTACTACTG |
| | CGCCCGGCGGGACTACTACCTCGGCAGCAACTCCTACGCGCTGGACTA |
| | CTGGGGCCAGGGCACCCTGGTGACCGTGAGCAGC |
| 7 | DIQMTQSPSSLSASVGDRVTITCQASQSVSNNLNWYQQKPGKAPKLLIYG |
| | ASKLEDGVPSRFSGSGSGTDFTFTISSLQPEDFATYYCLQHRYMWTFGQGT |
| | KVEIK |
| 8 | GACATCCAGATGACCCAGAGCCCCAGCAGCCTGAGCGCCAGCGTGGGC |
| | GACCGGGTGACCATCACCTGCCAGGCCAGCCAGAGCGTGAGCAACAA |
| | CCTGAACTGGTACCAGCAGAAGCCCGGCAAGGCCCCCAAGCTGCTGAT |
| | CTACGGCGCCAGCAAGCTGGAGGACGGCGTGCCCAGCCGGTTCAGCGG |
| | CAGCGGCAGCGGCACCGACTTCACCTTCACCATCAGCAGCCTGCAGCC |
| | CGAGGACTTCGCCACCTACTACTGCCTGCAGCACCGGTATATGTGGAC |
| | CTTCGGCCAGGGCACCAAGGTGGAGATCAAG |
| 9 | DIQMTQSPSSLSASVGDRVTITCQASQSVSNNLNWYQQKPGKAPKLLIYG |
| | ASKLEDGVPSRFSGSGSGTDFTFTISSLQPEDFATYYCQQHRFMWTFGQGT |
| | KVEIK |
| 10 | GACATCCAGATGACCCAGAGCCCCAGCAGCCTGAGCGCCAGCGTGGGC |
| | GACCGGGTGACCATCACCTGCCAGGCCAGCCAGAGCGTGAGCAACAA |
| | CCTGAACTGGTACCAGCAGAAGCCCGGCAAGGCCCCCAAGCTGCTGAT |
| | CTACGGCGCCAGCAAGCTGGAGGACGGCGTGCCCAGCCGGTTCAGCGG |
| | CAGCGGCAGCGGCACCGACTTCACCTTCACCATCAGCAGCCTGCAGCC |
| | CGAGGACTTCGCCACCTACTACTGCCAGCAGCACCGGTTCATGTGGAC |
| | CTTCGGCCAGGGCACCAAGGTGGAGATCAAG |
| 11 | DIQMTQSPSSLSASVGDRVTITCQASQSVSNNLNWYQQKPGKAPKLLIYG |
| | ASKLEDGVPSRFSGSGSGTDFTFTISSLQPEDFATYYCQQHRYMWTFGQG |
| | TKVEIK |
| 12 | GACATCCAGATGACCCAGAGCCCCAGCAGCCTGAGCGCCAGCGTGGGC |
| | GACCGGGTGACCATCACCTGCCAGGCCAGCCAGAGCGTGAGCAACAA |
| | CCTGAACTGGTACCAGCAGAAGCCCGGCAAGGCCCCCAAGCTGCTGAT |
| | CTACGGCGCCAGCAAGCTGGAGGACGGCGTGCCCAGCCGGTTCAGCGG |
| | CAGCGGCAGCGGCACCGACTTCACCTTCACCATCAGCAGCCTGCAGCC |
| | CGAGGACTTCGCCACCTACTACTGCCAGCAGCACAGGTACATGTGGAC |
| | CTTCGGCCAGGGCACCAAGGTGGAGATCAAG |
| 13 | EVQLVQSGAEVKKPGASVKVSCKASGYTFTGYYMNWVRQAPGQGLEW |
| | MGEINPATGSTTYNQKFKARVTITRDTSTSTAYMELSSLRSEDTAVYYCA |
| | RRDYYYGSGFYAMDYWGQGTLVTVSSGGGGSGGGGSGGGGSDIQMTQS |
| | PSSLSASVGDRVTITCQASQSVSNNLNWYQQKPGKAPKLLIYGASKLEDG |
| | VPSRFSGSGSGTDFTFTISSLQPEDFATYYCLQHRYLWTFGQGTKVEIK |
| 14 | EVQLVQSGAEVKKPGASVKVSCKASGYTFTGYYMNWVRQAPGQGLEW |
| | MGEINPATGSTTYNQKFKARVTITRDTSTSTAYMELSSLRSEDTAVYYCA |
| | RRDYYLGSNSYALDYWGQGTLVTVSSGGGGSGGGGSGGGGSDIQMTQSP |
| | SSLSASVGDRVTITCQASQSVSNNLNWYQQKPGKAPKLLIYGASKLEDGV |
| | PSRFSGSGSGTDFTFTISSLQPEDFATYYCLQHRYLWTFGQGTKVEIK |
| 15 | EVQLVQSGAEVKKPGASVKVSCKASGYTFTGYYMNWVRQAPGQGLEW |
| | MGEINPATGSTTYNQKFKARVTITRDTSTSTAYMELSSLRSEDTAVYYCA |
| | RRDYYYGSGFYAMDYWGQGTLVTVSSGGGGSGGGGSGGGGSDIQMTQS |
| | PSSLSASVGDRVTITCQASQSVSNNLNWYQQKPGKAPKLLIYGASKLEDG |
| | VPSRFSGSGSGTDFTFTISSLQPEDFATYYCLQHRYMWTFGQGTKVEIK |
| 16 | EVQLVQSGAEVKKPGASVKVSCKASGYTFTGYYMNWVRQAPGQGLEW |
| | MGEINPATGSTTYNQKFKARVTITRDTSTSTAYMELSSLRSEDTAVYYCA |
| | RRDYYYGSGFYAMDYWGQGTLVTVSSGGGGSGGGGSGGGGSDIQMTQS |
| | PSSLSASVGDRVTITCQASQSVSNNLNWYQQKPGKAPKLLIYGASKLEDG |
| | VPSRFSGSGSGTDFTFTISSLQPEDFATYYCQQHRFMWTFGQGTKVEIK |
| 17 | EVQLVQSGAEVKKPGASVKVSCKASGYTFTGYYMNWVRQAPGQGLEW |
| | MGEINPATGSTTYNQKFKARVTITRDTSTSTAYMELSSLRSEDTAVYYCA |
| | RRDYYYGSGFYAMDYWGQGTLVTVSSGGGGSGGGGSGGGGSDIQMTQS |
| | PSSLSASVGDRVTITCQASQSVSNNLNWYQQKPGKAPKLLIYGASKLEDG |
| | VPSRFSGSGSGTDFTFTISSLQPEDFATYYCQQHRYMWTFGQGTKVEIK |
| 18 | EVQLVQSGAEVKKPGASVKVSCKASGYTFTGYYMNWVRQAPGQGLEW |
| | MGEINPATGSTTYNQKFKARVTITRDTSTSTAYMELSSLRSEDTAVYYCA |
| | RRDYYLGSNSYALDYWGQGTLVTVSSGGGGSGGGGSGGGGSDIQMTQSP |
| | SSLSASVGDRVTITCQASQSVSNNLNWYQQKPGKAPKLLIYGASKLEDGV |
| | PSRFSGSGSGTDFTFTISSLQPEDFATYYCLQHRYMWTFGQGTKVEIK |
| 19 | EVQLVQSGAEVKKPGASVKVSCKASGYTFTGYYMNWVRQAPGQGLEW |
| | MGEINPATGSTTYNQKFKARVTITRDTSTSTAYMELSSLRSEDTAVYYCA |
| | RRDYYLGSNSYALDYWGQGTLVTVSSGGGGSGGGGSGGGGSDIQMTQSP |
| | SSLSASVGDRVTITCQASQSVSNNLNWYQQKPGKAPKLLIYGASKLEDGV |
| | PSRFSGSGSGTDFTFTISSLQPEDFATYYCQQHRFMWTFGQGTKVEIK |
| 20 | MALPVTALLLPLALLLHAARP |
| 21 | TTTPAPRPPTPAPTIASQPLSLRPEACRPAAGGAVHTRGLDFACD |
| 22 | FWVLVVVGGVLACYSLLVTVAFIIFWV |
| 23 | RSKRSRLLHSDYMNMTPRRPGPTRKHYQPYAPPRDFAAYRS |
| 24 | RVKFSRSADAPAYQQGQNQLYNELNLGRREEYDVLDKRRGRDPEMGGK |
| | PQRRKNPQEGLYNELQKDKMAEAYSEIGMKGERRRGKGHDGLYQGLST |
| | ATKDTYDALHMQALPPR |
| 25 | IYIWAPLAGTCGVLLLSLVIT |
| 26 | KRGRKKLLYIFKQPFMRPVQTTQEEDGCSCRFPEEEEGGCEL |
| 27 | PKSCDKTHTCP |
| 28 | GQPREPQVYTLPPSRDELTKNQVSLTCLVKGFYPSDIAVEWESNGQPENN |
| | YKTTPPVLDSDGSFFLYSKLTVDKSRWQQGNVFSCSVMHEALHNHYTQK |
| | SLSLSPGK |
| 29 | ESKYGPPCPSCP |
| 30 | ESKYGPPCPPCP |
| 31 | MASMGLQVMGIALAVLGWLAVMLCCALPMWRVTAFIGSNIVTSQTIWE |
| | GLWMNCVVQSTGQMQCKVYDSLLALPQDLQAARALVIISIIVAALGVLLS |
| | VVGGKCTNCLEDESAKAKTMIVAGVVFLLAGLMVIVPVSWTAHNIIQDFY |
| | NPLVASGQKREMGASLYVGWAASGLLLLGGGLLCCNCPPRTDKPYSAKY |
| | SAARSAAASNYV |
| 32 | MASAGMQILGVVLTLLGWVNGLVSCALPMWKVTAFIGNSIVVAQVVWE |
| | GLWMSCVVQSTGQMQCKVYDSLLALPQDLQAARALCVIALLVALFGLLV |
| | YLAGAKCTTCVEEKDSKARLVLTSGIVFVISGVLTLIPVCWTAHAIIRDFY |
| | NPLVAEAQKRELGASLYLGWAASGLLLLGGGLLCCTCPSGGSQGPSHYM |
| | ARYSTSAPAISRGPSEYPTKNYV |
| 33 | MASTGLELLGMTLAVLGWLGTLVSCALPLWKVTAFIGNSIVVAQVVWEG |
| | LWMSCVVQSTGQMQCKVYDSLLALPQDLQAARALCVIALLLALLGLLVA |
| | ITGAQCTTCVEDEGAKARIVLTAGVILLLAGILVLIPVCWTAHAIIQDFYNP |
| | LVAEALKRELGASLYLGWAAAALLMLGGGLLCCTCPPPQVERPRGPRLG |
| | YSIPSRSGASGLDKRDYV |
| 34 | DKTHTCPPCPAPELLGGPSVFLFPPKPKDTLMISRTPEVTCVVVDVSHEDP |
| | EVKFNWYVDGVEVHNAKTKPREEQYNSTYRVVSVLTVLHQDWLNGKEY |
| | KCKVSNKALPAPIEKTISKAKGQPREPQVYTLPPSRDELTKNQVSLTCLVK |
| | GFYPSDIAVEWESNGQPENNYKTTPPVLDSDGSFFLYSKLTVDKSRWQQG |
| | NVFSCSVMHEALHNHYTQKSLSLSPGK |
| 35 | GYYMN |
| 36 | EINPATGSTTYNQKFKA |
| 37 | RDYYYGSGFYAMDY |
| 38 | QASQSVSNNLN |
| 39 | GASKLED |
| 40 | LQHRYLWT |
| 41 | RDYYLGSNSYALDY |
| 42 | LQHRYMWT |
| 43 | QQHRFMWT |
| 44 | QQHRYMWT |
| 45 | FWVLVVVGGVLACYSLLVTVAFIIFWVRSKRSRLLHSDYMNMTPRRPGPT |
| | RKHYQPYAPPRDFAAYRSRVKFSRSADAPAYQQGQNQLYNELNLGRREE |
| | YDVLDKRRGRDPEMGGKPQRRKNPQEGLYNELQKDKMAEAYSEIGMKG |
| | ERRRGKGHDGLYQGLSTATKDTYDALHMQALPPR |
| 46 | IYIWAPLAGTCGVLLLSLVITLYCKRGRKKLLYIFKQPFMRPVQTTQEEDG |
| | CSCRFPEEEEGGCELRVKFSRSADAPAYQQGQNQLYNELNLGRREEYDVL |
| | DKRRGRDPEMGGKPQRRKNPQEGLYNELQKDKMAEAYSEIGMKGERRR |
| | GKGHDGLYQGLSTATKDTYDALHMQALPPR |
| 47 | FWVLVVVGGVLACYSLLVTVAFIIFWVRSKRSRLLHSDYMNMTPRRPGPT |
| | RKHYQPYAPPRDFAAYRSKRGRKKLLYIFKQPFMRPVQTTQEEDGCSCRF |
| | PEEEEGGCELRVKFSRSADAPAYQQGQNQLYNELNLGRREEYDVLDKRR |
| | GRDPEMGGKPQRRKNPQEGLYNELQKDKMAEAYSEIGMKGERRRGKGH |
| | DGLYQGLSTATKDTYDALHMQALPPR |
| 48 | EVQLVQSGAEVKKPGASVKVSCKASGYTFTGYYMNWVRQAPGQGLEW |
| | MGEINPATGSTTYNQKFKARVTITRDTSTSTAYMELSSLRSEDTAVYYCA |
| | RRDYYYGSGFYAMDYWGQGTLVTVSSGGGGSGGGGSGGGGSDIQMTQS |
| | PSSLSASVGDRVTITCQASQSVSNNLNWYQQKPGKAPKLLIYGASKLEDG |
| | VPSRFSGSGSGTDFTFTISSLQPEDFATYYCLQHRYLWTFGQGTKVEIKTT |
| | TPAPRPPTPAPTIASQPLSLRPEACRPAAGGAVHTRGLDFACDFWVLVVV |
| | GGVLACYSLLVTVAFIIFWVRSKRSRLLHSDYMNMTPRRPGPTRKHYQPY |
| | APPRDFAAYRSRVKFSRSADAPAYQQGQNQLYNELNLGRREEYDVLDKR |
| | RGRDPEMGGKPQRRKNPQEGLYNELQKDKMAEAYSEIGMKGERRRGKG |
| | HDGLYQGLSTATKDTYDALHMQALPPR |
| 49 | EVQLVQSGAEVKKPGASVKVSCKASGYTFTGYYMNWVRQAPGQGLEW |
| | MGEINPATGSTTYNQKFKARVTITRDTSTSTAYMELSSLRSEDTAVYYCA |
| | RRDYYYGSGFYAMDYWGQGTLVTVSSGGGGSGGGGSGGGGSDIQMTQS |
| | PSSLSASVGDRVTITCQASQSVSNNLNWYQQKPGKAPKLLIYGASKLEDG |
| | VPSRFSGSGSGTDFTFTISSLQPEDFATYYCQQHRYMWTFGQGTKVEIKTT |
| | TPAPRPPTPAPTIASQPLSLRPEACRPAAGGAVHTRGLDFACDFWVLVVV |
| | GGVLACYSLLVTVAFIIFWVRSKRSRLLHSDYMNMTPRRPGPTRKHYQPY |
| | APPRDFAAYRSRVKFSRSADAPAYQQGQNQLYNELNLGRREEYDVLDKR |
| | RGRDPEMGGKPQRRKNPQEGLYNELQKDKMAEAYSEIGMKGERRRGKG |
| | HDGLYQGLSTATKDTYDALHMQALPPR |
| 50 | EVQLQQSGPELVKPGASMKISCKASGYSFTGYTMNWVKQSHGKNLEWIG |
| | LINPYNGGTIYNQKFKGKATLTVDKSSSTAYMELLSLTSEDSAVYYCARD |
| | YGFVLDYWGQGTTLTVSSGGGGSGGGGSGGGGSQIVLTQSPSIMSVSPGE |
| | KVTITCSASSSVSYMHWFQQKPGTSPKLSIYSTSNLASGVPARFSGRGSGT |
| | SYSLTISRVAAEDAATYYCQQRSNYPPWTFGGGTKLEIK |
| 51 | EVQLQQSGPELVKPGASMKISCKASGYSFTGYTMNWVKQSHGKNLEWIG |
| | LINPYNGGTIYNQKFKGKATLTVDKSSSTAYMELLSLTSEDSAVYYCARD |
| | YGFVLDYWGQGTTLTVSSGGGGSGGGGSGGGGSQIVLTQSPSIMSVSPGE |
| | KVTITCSASSSVSYMHWFQQKPGTSPKLSIYSTSNLASGVPARFSGRGSGT |
| | SYSLTISRVAAEDAATYYCQQRSNYPPWTFGGGTKLEIKTTTPAPRPPTPA |
| | PTIASQPLSLRPEACRPAAGGAVHTRGLDFACDFWVLVVVGGVLACYSLL |
| | VTVAFIIFWVRSKRSRLLHSDYMNMTPRRPGPTRKHYQPYAPPRDFAAYR |
| | SRVKFSRSADAPAYQQGQNQLYNELNLGRREEYDVLDKRRGRDPEMGG |
| | KPQRRKNPQEGLYNELQKDKMAEAYSEIGMKGERRRGKGHDGLYQGLS |
| | TATKDTYDALHMQALPPR |
| 52 | RDYYX₁GSX₂X₃YAX₄DY |
| 53 | X₅QHRX₆X₇WT |

1. (canceled)

2. An antibody that recognizes CLDN6 or an antigen-binding fragment thereof, selected from the group consisting of:
(1) The antibody or antigen-binding fragment comprises a heavy chain variable region, wherein the heavy chain variable region comprises HCDR1 shown in SEQ ID NO: 35, and/or HCDR2 shown in SEQ ID NO: 36, and/or HCDR3 shown in SEQ ID NO: 37 or 41;
(2) The antibody or antigen-binding fragment comprises a light chain variable region, wherein the light chain variable region comprises LCDR1 shown in SEQ ID NO: 38, and/or LCDR2 shown in SEQ ID NO: 39, and/or LCDR3 shown in SEQ ID NO: 40, 42, 43, or 44;
(3) The antibody or antigen-binding fragment comprises the heavy chain variable region of (1) and the light chain variable region of (2);
(4) The antibody or antigen-binding fragment is a variant of any one of (1) to (3), wherein the variant comprises at least one but no more than 7, 6, 5, 4, 3, or 2 amino acid changes in total on 1, 2, 3, 4, 5, or 6 CDR regions, and maintains the same or similar activity as the antibody or antigen-binding fragment of any one of (1) to (3).

3. The antibody or antigen-binding fragment of claim 2, selected from the group consisting of:
(1) The antibody or antigen-binding fragment comprises HCDR1 shown in SEQ ID NO: 35, HCDR2 shown in SEQ ID NO: 36, HCDR3 shown in SEQ ID NO: 37, LCDR1 shown in SEQ ID NO: 38, LCDR2 shown in SEQ ID NO: 39, and LCDR3 shown in SEQ ID NO: 40; or
(2) The antibody or antigen-binding fragment comprises HCDR1 shown in SEQ ID NO: 35, HCDR2 shown in SEQ ID NO: 36, HCDR3 shown in SEQ ID NO: 41, LCDR1 shown in SEQ ID NO: 38, LCDR2 shown in SEQ ID NO: 39, and LCDR3 shown in SEQ ID NO: 40; or
(3) The antibody or antigen-binding fragment comprises HCDR1 shown in SEQ ID NO: 35, HCDR2 shown in SEQ ID NO: 36, HCDR3 shown in SEQ ID NO: 37, LCDR1 shown in SEQ ID NO: 38, LCDR2 shown in SEQ ID NO: 39, and LCDR3 shown in SEQ ID NO: 42; or
(4) The antibody or antigen-binding fragment comprises HCDR1 shown in SEQ ID NO: 35, HCDR2 shown in SEQ ID NO: 36, HCDR3 shown in SEQ ID NO: 37, LCDR1 shown in SEQ ID NO: 38, LCDR2 shown in SEQ ID NO: 39, and LCDR3 shown in SEQ ID NO: 43; or
(5) The antibody or antigen-binding fragment comprises HCDR1 shown in SEQ ID NO: 35, HCDR2 shown in SEQ ID NO: 36, HCDR3 shown in SEQ ID NO: 41, LCDR1 shown in SEQ ID NO: 38, LCDR2 shown in SEQ ID NO: 39, and LCDR3 shown in SEQ ID NO: 42; or
(6) The antibody or antigen-binding fragment comprises HCDR1 shown in SEQ ID NO: 35, HCDR2 shown in SEQ ID NO: 36, HCDR3 shown in SEQ ID NO: 41, LCDR1 shown in SEQ ID NO: 38, LCDR2 shown in SEQ ID NO: 39, and LCDR3 shown in SEQ ID NO: 43; or
(7) The antibody or antigen-binding fragment comprises HCDR1 shown in SEQ ID NO: 35, HCDR2 shown in SEQ ID NO: 36, HCDR3 shown in SEQ ID NO: 37, LCDR1 shown in SEQ ID NO: 38, LCDR2 shown in SEQ ID NO: 39, and LCDR3 shown in SEQ ID NO: 44; or
(8) The antibody or antigen-binding fragment is a variant of any one of (1) to (7), wherein the variant comprises at least one but no more than 7, 6, 5, 4, 3, or 2 amino acid changes in total on 1, 2, 3, 4, 5, or 6 CDR regions, and maintains the same or similar activity as the antibody or antigen-binding fragment of any one of (1) to (7).

4. The antibody or an antigen-binding fragment of claim 2, selected from the group consisting of:
(1) The antibody or antigen-binding fragment comprises a heavy chain variable region, wherein the heavy chain variable region comprises the amino acid sequence shown in SEQ ID NO: 1 or 5, or an amino acid sequence with at least 80%, 85%, 90%, 91%, 92%, 93%, 94%, 95%, 96%, 97%, 98%, or 99% identity thereto;
(2) The antibody or antigen-binding fragment comprises a light chain variable region, wherein the light chain variable region comprises the amino acid sequence shown in SEQ ID NO: 3, 7, 9 or 11, or an amino acid sequence with at least 80%, 85%, 90%, 91%, 92%, 93%, 94%, 95%, 96%, 97%, 98%, or 99% identity thereto;
(3) The antibody or antigen-binding fragment comprises the heavy chain variable region of (1) and the light chain variable region of (2);
(4) The antibody or antigen-binding fragment is a variant of any one of (1) to (3), wherein the variant comprises at least one but no more than 20, 15, 10, 9, 8, 7, 6, 5, 4, 3 or 2 amino acid changes on VH or VL, and maintains the same or similar activity as the antibody or antigen-binding fragment of any one of (1) to (3); Preferably, the antibody or antigen-binding fragment is selected from the group consisting of;
(1) the heavy chain variable region of the antibody or antigen-binding fragment comprises the amino acid sequence shown in SEQ ID NO: 1, or an amino acid sequence with at least 80%, 85%, 90%, 91%, 92%, 93%, 94%, 95%, 96%, 97%, 98%, or 99% identity thereto; and the light chain variable region of the antibody or antigen-binding fragment comprises the amino acid sequence shown in SEQ ID NO. 3, or an amino acid sequence with at least 80%, 85%, 90%, 91%, 92%, 93%, 94%, 95%, 96%, 97%, 98%, or 99% identity thereto; or
(2) the heavy chain variable region of the antibody or antigen-binding fragment comprises the amino acid sequence shown in SEQ ID NO: 2, or an amino acid sequence with at least 80%, 85%, 90%, 91%, 92%, 93%, 94%, 95%, 96%, 97%, 98%, or 99% identity thereto; and the light chain variable region of the antibody or antigen-binding fragment comprises the amino acid sequence shown in SEQ ID NO: 3, or an amino acid sequence with at least 80%, 85%, 90%, 91%, 92%, 93%, 94%, 95%, 96%, 97%, 98%, or 99% identity thereto; or
(3) the heavy chain variable region of the antibody or antigen-binding fragment comprises the amino acid sequence shown in SEQ ID NO: 1, or an amino acid sequence with at least 80%, 85%, 90%, 91%, 92%, 93%, 94%, 95%, 96%, 97%, 98%, or 99% identity thereto; and the light chain variable region of the antibody or antigen-binding fragment comprises the amino acid sequence shown in SEQ ID NO: 7, or an amino acid sequence with at least 80%, 85%, 90%, 91%, 92%, 93%, 94%, 95%, 96%, 97%, 98%, or 99% identity thereto; or
(4) the heavy chain variable region of the antibody or antigen-binding fragment comprises the amino acid sequence shown in SEQ ID NO: 1, or an amino acid sequence with at least 80%, 85%, 90%, 91%, 92%, 93%, 94%, 95%, 96%, 97%, 98%, or 99% identity thereto; and the light chain variable region of the antibody or antigen-binding fragment comprises the amino acid sequence shown in SEQ ID NO: 9, or an amino acid sequence with at least 80%, 85%, 90%, 91%, 92%, 93%, 94%, 95%, 96%, 97%, 98%, or 99% identity thereto; or
(5) the heavy chain variable region of the antibody or antigen-binding fragment comprises the amino acid sequence shown in SEQ ID NO: 5, or an amino acid sequence with at least 80%, 85%, 90%, 91%, 92%, 93%, 94%, 95%, 96%, 97%, 98%, or 99% identity thereto; and the light chain variable region of the antibody or antigen-binding fragment comprises the amino acid sequence shown in SEQ ID NO: 7, or an amino acid sequence with at least 80%, 85%, 90%, 91%, 92%, 93%, 94%, 95%, 96%, 97%, 98%, or 99% identity thereto; or
(6) the heavy chain variable region of the antibody or antigen-binding fragment comprises the amino acid sequence shown in SEQ ID NO: 5, or an amino acid sequence with at least 80%, 85%, 90%, 91%, 92%, 93%, 94%, 95%, 96%, 97%, 98%, or 99% identity thereto; and the light chain variable region of the antibody or antigen-binding fragment comprises the amino acid sequence shown in SEQ ID NO: 9, or an amino acid sequence with at least 80%, 85%, 90%, 91%, 92%, 93%, 94%, 95%, 96%, 97%, 98%, or 99% identity thereto; or
(7) the heavy chain variable region of the antibody or antigen-binding fragment comprises the amino acid sequence shown in SEQ ID NO: 1, or an amino acid sequence with at least 80%, 85%, 90%, 91%, 92%, 93%, 94%, 95%, 96%, 97%, 98%, or 99% identity thereto; and the light chain variable region of the antibody or antigen-binding fragment comprises the amino acid sequence shown in SEQ ID NO: 11, or an amino acid sequence with at least 80%, 85%, 90%, 91%, 92%, 93%, 94%, 95%, 96%, 97%, 98%, or 99% identity thereto; or
(8) The antibody or antigen-binding fragment is a variant of any of (1) to (7), wherein the variant comprises at least one but no more than 20, 15, 10, 9, 8, 7, 6, 5, 4, 3, or 2 amino acid changes on VH or VL, and maintains the same or similar activity as the antibody or antigen-binding fragment of any one of (1) to (7).

5. (canceled)

6. The antibody or an antigen-binding fragment of claim 2, wherein the antibody or antigen-binding fragment comprises a heavy chain variable region and a light chain variable region, the light chain variable region comprises the amino acid sequence shown in SEQ ID NO: 3, 7, 9 or 11, or an amino acid sequence with at least 80%, 85%, 90%, 91%, 92%, 93%, 94%, 95%, 96%, 97%, 98%, or 99% identity thereto; and
the heavy chain variable region comprises HCDR1 shown in SEQ ID NO: 35, HCDR2 shown in SEQ ID NO: 36, and HCDR3 shown in SEQ ID NO: 37 or 41; Preferably the heavy chain variable region comprises the amino acid sequence shown in SEQ ID NO: 1 or 5, or an amino acid sequence with at least 80%, 85%, 90%, 91%, 92%, 93%, 94%, 95%, 96%, 97%, 98%, or 99% identity thereto.

7. (canceled)

8. The antibody or an antigen-binding fragment of claim 2, wherein the antibody or antigen-binding fragment comprises a heavy chain variable region and a light chain variable region, the heavy chain variable region comprises the amino acid sequence shown in SEQ ID NO: 1 or 5, or an amino acid sequence with at least 80%, 85%, 90%, 91%, 92%, 93%, 94%, 95%, 96%, 97%, 98%, or 99% identity thereto; and
the light chain variable region comprises LCDR1 shown in SEQ ID NO: 38, LCDR2 shown in SEQ ID NO: 39, and LCDR3 shown in SEQ ID NO: 40, 42, 43 or 44; preferably the light chain variable region comprises the amino acid sequence shown in SEQ ID NO: 3, 7, 9 or 11, or an amino acid sequence with at least 80%, 85%, 90%, 91%, 92%, 93%, 94%, 95%, 96%, 97%, 98%, or 99% identity thereto.

9-10. (canceled)

11. The antibody or antigen-binding fragment of claim 2,
comprising an amino acid sequence shown in SEQ ID NO: 13, 14, 15, 16, 17, 18, or 19, or an amino acid sequence having at least 80%, 85%, 90%, 91%, 92%, 93%, 94%, 95%, 96%, 97%, 98%, or 99% identity thereto, or comprises at least one but no more than 20, 15, 10, 9, 8, 7, 6, 5, 4, 3, or 2 amino acid changes in the sequences as said above.

12. The antibody or antigen-binding fragment of claim 2, wherein the antibody or antigen-binding fragment binds to CLDN6 without significantly binding to CLDN4 or CLDN9; and/or, the antibody or antigen-binding fragment binds to cells expressing CLDN6 without significantly binding to cells expressing CLDN4, CLDN9, or a combination thereof.

13. An immunoconjugate, comprising: the antibody or antigen-binding fragment of claim 2, and a functional molecule linked thereto.

14. A chimeric receptor comprising an extracellular region, wherein the extracellular region comprises the antibody or antigen-binding fragment of claim 2;
the chimeric receptor comprises: a chimeric antigen receptor (CAR), a chimeric T-cell receptor, a T-cell antigen coupler (TAC), a synthetic polypeptide receptor (synNotch), or a combination thereof.

15. The chimeric receptor of claim 14, wherein the chimeric receptor is a chimeric antigen receptor (CAR), which comprises the antibody or antigen-binding fragment, a transmembrane region, and an intracellular signaling region; and preferably, the antibody or antigen-binding fragment is linked to the transmembrane region via a hinge domain.

16-20. (canceled)

21. The chimeric receptor of claim 14, wherein the chimeric receptor comprises:
An antibody or antigen-binding fragment of thereof, a transmembrane region of CD8/CD28, and CD3ζ; or
An antibody or antigen-binding fragment of thereof, a transmembrane region of CD8/CD28, an intracellular signaling region of CD137, and CD3ζ; or
An antibody or antigen-binding fragment of thereof, a transmembrane region of CD8/CD28, an intracellular signaling region of CD28, and CD3ζ; or
An antibody or antigen-binding fragment of thereof, a transmembrane region of CD8/CD28, an intracellular signaling region of CD28, an intracellular signaling region of CD137, and CD3ζ.

22. The chimeric receptor of claim 21, wherein the chimeric receptor comprises an amino acid sequence formed by linking any one of SEQ ID NOs: 13, 14, 15, 16, 17, 18, or 19 with any one of SEQ ID NOs: 45, 46, or 47 respectively, or an amino acid sequence having at least 80%, 85%, 90%, 95%, 96%, 97%, 98%, or 99% sequence identity to the sequences as said above;
preferably, the chimeric receptor comprises the sequence shown in SEQ ID NO: 48 or 49, or an amino acid sequence having at least 80%, 85%, 90%, 95%, 96%, 97%, 98%, or 99% sequence identity to the sequences as said above.

23. A nucleic acid encoding the antibody or antigen-binding fragment of claim 2, an immunoconjugate, or the chimeric receptor, wherein the immunoconjugate comprises the antibody or antigen-binding fragment and a functional molecule linked thereto; and the chimeric receptor comprises an extracellular region, wherein the extracellular region comprises the antibody or antigen-binding fragment.

24. A vector comprising the nucleic acid of claim 23.

25. A cell comprising the antibody or antigen-binding fragment of claim 2, an immunoconjugate, chimeric receptor, a nucleic acid, and/or a vector, wherein the immunoconjugate comprises the antibody or antigen-binding fragment and a functional molecule linked thereto; the chimeric receptor comprises an extracellular region, wherein the extracellular region comprises the antibody or antigen-binding fragment; the nucleic acid encoding the antibody or antigen-binding fragment, the immunoconjugate, or the chimeric receptor, and the vector comprising the nucleic acid; preferably wherein the cells comprise T cells, natural killer cells, natural killer T cells, NK92 cells, cytotoxic T cells, dendritic cells, macrophages, cytokine-induced killer (CIK) cells, pluripotent stem cells, stem cell-derived immune cells, or combinations thereof.

26-29. (canceled)

30. A pharmaceutical composition, comprising the antibody or antigen-binding fragment of claim 2, an immunoconjugate, a chimeric receptor, a nucleic acid, a vector and/or a cell, and a pharmaceutically acceptable adjuvant;
Wherein the immunoconjugate comprises the antibody or antigen-binding fragment and a functional molecule linked thereto; the chimeric receptor comprises an extracellular region, wherein the extracellular region comprises the antibody or antigen-binding fragment; the nucleic acid encoding the antibody or antigen-binding fragment, the immunoconjugate, or the chimeric receptor; and the vector comprising the nucleic acid; and the cell comprises the antibody or antigen-binding fragment, immunoconjugate, and/or chimeric receptor.

31. A combined administration, wherein the antibody or antigen-binding fragment of claim 2; an immunoconjugate; a chimeric receptor; a cell; the pharmaceutical composition with an agent that enhances their function are co-administered,
wherein the immunoconjugate comprises the antibody or antigen-binding fragment and a functional molecule linked thereto; the chimeric receptor comprises an extracellular region, wherein the extracellular region comprises the antibody or antigen-binding fragment; the cell comprises the antibody or antigen-binding fragment, the immunoconjugate, and/or chimeric receptor; and the pharmaceutical composition comprises the antibody or antigen-binding fragment, immunoconjugate, chimeric receptor, nucleic acid, vector and/or cell, and a pharmaceutically acceptable adjuvant;
preferably, co-administered with a chemotherapeutic agent;
and/or co-administered with an agent that mitigates one or more related side effects;
and/or co-administered with cells expressing a chimeric antigen receptor targeting a molecule other than CLDN6;
and/or co-administered with an agent for treating diseases associated with CLDN6 expression;
preferably, the agent comprises an antibody or antigen-binding fragment, cells, RNA, a vaccine, an oncolytic virus, a checkpoint inhibitor, a BTK inhibitor, a chemical medicament, a radiotherapy agent, a hormonal therapy agent, a toxin, an immunotherapeutic agent, or a combination thereof.

32. (canceled)

33. A kit, comprising the antibody or antigen-binding fragment of claim 2, an immunoconjugate, a chimeric receptor, a nucleic acid, a vector, a cell, and/or a pharmaceutical composition;
wherein the immunoconjugate comprises the antibody or antigen-binding fragment and a functional molecule linked thereto; the chimeric receptor comprises an extracellular region, wherein the extracellular region comprises the antibody or antigen-binding fragment; the nucleic acid encoding the antibody or antigen-binding fragment, immunoconjugate, or chimeric receptor; the vector comprising the nucleic acid; the cell comprises the antibody or antigen-binding fragment, immunoconjugate, chimeric receptor, nucleic acid and/or vector; and the pharmaceutically composition comprises the antibody or antigen-binding fragment, immunoconjugate, chimeric receptor, nucleic acid, vector, and/or cell, and a pharmaceutically acceptable adjuvant.

34-35. (canceled)

36. A method for treating/diagnosing a disease, comprising administering to a subject in need thereof an effective amount of the antibody or antigen-binding fragment of claim 2, an immunoconjugate, a cell, a pharmaceutical composition, or a kit;
wherein the immunoconjugate comprises the antibody or antigen-binding fragment and a functional molecule linked thereto;
the cell comprises the antibody or antigen-binding fragment, immunoconjugate, chimeric receptor, nucleic acid, and/or vector; and
the pharmaceutical composition comprises the antibody or antigen-binding fragment, immunoconjugate, chimeric receptor, nucleic acid, vector, and/or cell, and a pharmaceutically acceptable adjuvant,
the kit comprises the antibody or antigen-binding fragment, immunoconjugate, chimeric receptor, nucleic acid, vector, cell, and/or pharmaceutical composition;
preferably, the disease is selected from inflammatory disorders, infections, autoimmune diseases, or tumors;
preferably, the subject is a human;
preferably, the cell is an autologous or allogeneic T cell for the subject.

37. The antibody or antigen-binding fragment of claim 2, an immunoconjugate, a cell, a pharmaceutical composition, and/or a kit, for use in the treatment/diagnosis of a disease expressing CLDN6; preferably, the disease is selected from inflammatory disorders, infections, autoimmune diseases, or tumors; more preferably, the tumor is a solid tumor; more preferably, the tumor is ovarian cancer, breast cancer, cervical cancer, gastric cancer, lung cancer, testicular cancer, germ cell and embryonic tumors, ovarian epithelial carcinoma, non-small cell lung cancer, non-squamous non-small cell lung cancer, endometrial cancer, or a combination thereof,
wherein the immunoconjugate comprises the antibody or antigen-binding fragment and a functional molecule linked thereto;
the cell comprises the antibody or antigen-binding fragment, immunoconjugate, chimeric receptor, nucleic acid, and/or vector;
the pharmaceutical composition comprises the antibody or antigen-binding fragment, immunoconjugate, chimeric receptor, nucleic acid, vector, and/or cell, and a pharmaceutically acceptable adjuvant; and
the kit comprises the antibody or antigen-binding fragment, immunoconjugate, chimeric receptor, nucleic acid, vector, cell, and/or pharmaceutical composition.
